(19) United States
(12) Patent Application Publication
(10) Pub. No.: US 2024/0279687 A1
(43) Pub. Date: Aug. 22, 2024

(54) PEPTIDE NUCLEIC ACIDS FOR SPATIOTEMPORAL CONTROL OF CRISPR-CAS BINDING

(71) Applicant: Yale University, New Haven, CT (US)

(72) Inventors: Nicholas Economos, New Haven, CT (US); Elias Quijano, Durham, CT (US); Peter Glazer, Guilford, CT (US)

(21) Appl. No.: 18/567,662

(22) PCT Filed: Jun. 7, 2022
(86) PCT No.: PCT/US2022/032532

§ 371 Date: Dec. 6, 2023

Related U.S. Application Data
(60) Provisional application No. 63/197,879, filed on Jun. 7, 2021.

(51) Int. Cl. C12N 15/90; C12N 9/22; C12N 15/11; C12N 15/113

(52) U.S. Cl. CPC C12N 15/907; C12N 9/22; C12N 15/11; C12N 15/111; C12N 15/113; C12N 2310/113; C12N 2310/20; C12N 2310/3181; C12N 2310/3519; C12N 2800/80

(57) ABSTRACT

Compositions and methods for modulating the localization and/or activity of CRISPR/Cas systems are provided. Typically, the compositions include a single stranded peptide nucleic acid (ssPNA) oligomer having a nucleobase sequence that hybridizes to part or all of the spacer sequence of CRISPR RNA such as a crRNA, gRNA, sgRNA, etc., to form a PNA:RNA duplex. The methods typically include contacting the cells with an effective amount of the ssPNA oligomer in conjunction with the elements of a CRISPR/Cas system to modify CRISPR/Cas activity at a desired target. Strategies and methods of use there for using the compositions to modulate on-target and/or off-target editing are provided.

### CROSS-REFERENCE TO RELATED APPLICATION

This application claims the benefit of and priority to U.S. Ser. No. 63/197,879, filed on Jun. 7, 2021, which is incorporated by reference herein in its entirety.

### STATEMENT REGARDING FEDERALLY SPONSORED RESEARCH OR DEVELOPMENT

This invention was made with government support under HL149185 and GM007205 awarded by National Institutes of Health. The government has certain rights in the invention.

### REFERENCE TO SEQUENCE LISTING

The Sequence Listing submitted as a text file named “YU_8103_PCT_ST25.txt,” created on Jun. 1, 2022, and having a size of 16,384 bytes is hereby incorporated by reference pursuant to 37 C.F.R. § 1.52(e)(5).

### FIELD OF THE INVENTION

The field of the invention is generally related to spatial and temporal modification of CRISPR/Cas activity.

### BACKGROUND OF THE INVENTION

Off-target cleavage events are well-characterized consequences of bacteria-derived nucleases and threaten their safe application to human therapy (Hsu, et al. Nature Biotechnology, 31, 827-832 (2013); Zhang, et al., Molecular Therapy—Nucleic Acids, 4, e264 (2015)). Additionally, as Cas9-based biotechnologies continue to rapidly develop across sectors (Adli, Nature Communications, 9, 1911(2018); Doudna and Charpentier, Science, 346, 1258096. (2014)), there is a need for modulating tools to further realize the potential of these systems and conveniently manipulate dose, timing, and accuracy (Shivram, et al. Nature Chemical Biology, 17, 10-19 (2021)). Preferably such tools would feature user-friendly design and application, facile delivery, scalable synthesis, and sequence specificity for application to multiplexed experiments.

“Anti-CRISPR” refers to molecules and modulators of CRISPR-Cas9 activity for spatiotemporal control of genome targeting to improve precision and safety (Pawluk, et al., Nat Rev Microbiol, 16, 12-17 (2018), doi.org/10.1038/nrmicro.2017.120; Marino, et al., Nat Methods 17, 471-479 (2020), doi.org/10.1038/s41592-020-0771-6, Dolgin, Nature 577, 308-310 (2020), doi.org/10.1038/d41586-020-00053-0). Therapeutic and research applications include reducing off-target cutting and large-scale recombination, tighter control, including of dCas fusions used to target activity of other factors anywhere in the genome. The tighter spatiotemporal control serves as a molecular safety net, and can be used to augment and/or expand virtually all current CRISPR-Cas methodologies.

Current technologies include anti-CRISPR proteins and off-target “shields.” Anti-CRIPSR proteins are phage derived factors that strongly inhibit Cas enzymes (Cas family specific) (Pawluk, et al., Nat Rev Microbiol, 16, 12-17 (2018), doi.org/10.1038/nrmicro.2017.120; Marino, et al., Nat Methods 17, 471-479 (2020), doi.org/10.1038/s41592-020-0771-6). A strong, diverse class of inhibitors, they can reduce off-target cutting when introduced after short window of Cas activity (Shin, et al., Science Advances, 3(7):e1701620 (2017, doi.org/10.1126/sciadv.1701620). However, anti-CRISPR proteins are potentially immunogenic and toxic viral-derived proteins, and are difficult cargo for delivery into cells.

Off-target shields refers to a recent field of nuclease-deficient Cas9 guide RNAs that can “protect” known off-targets from cleavage (Rose, et al., Nat Commun 11, 2697 (2020), doi.org/10.1038/s41467-020-16542-9, Coelho, et al., Nat Commun 11, 4132 (2020), doi.org/10.1038/s41467-020-17952-5). However, these modulators require the addition of more Cas9, have limited application, and design restrictions.

As a countermeasure to deleterious events, and to improve control over Cas9-derived systems, previous studies explored the application of anti-CRISPR (Acr) proteins and small molecules to directly bind and inhibit Cas9 and related CRISPR family proteins (Shin, et al., Science Advances, 3, e1701620.-8 (2017); Marino, et al. Nature Methods, 17, 471-479 (2020)). However, anti-CRISPR systems feature notable limitations including toxicity and potential immunogenicity related to peptide expression, generalized inhibition mechanisms, modest potency, and restriction to a single Cas ortholog or CRISPR subtype (Marino, et al., Science, 362, 240 (2018); Meeske, et al. Science, 369, 54 (2020)).

Nucleic acid-based inhibitors such as DNA aptamers and chemically modified gRNA-binding nucleic acids have also been explored with some success. In both cases, however, sugar-phosphate-backboned nucleic acids were only able to appreciably bind the generalized PAM-recognition components of gRNAs rather than the full guide spacer sequence, and thus lack true sequence-specificity.

Finally, other Cas9 fusions and inducible versions of Cas9 have been proposed to have improved temporal control of targeting (on/off switch).

However, there remains a need for additional approaches that effectively address the need to control the timing, duration, efficiency, and specificity of CRISPR-Cas9 systems and their wide-ranging applications (Shivram, et al., Nature Chemical Biology, 17, 10-19, (2021), doi:10.1038/s41589-020-00700-7).

Thus, it is an object of the invention to provide alternative anti-CRISPR compositions and methods of use thereof for modulation of CRISPR/Cas-related gene editing and other applications.

### SUMMARY OF THE INVENTION

Compositions and methods for modulating the localization and/or activity of CRISPR/Cas systems are provided. Typically, the compositions include a single stranded peptide nucleic acid (ssPNA) oligomer having a nucleobase sequence that hybridizes to part or all of the spacer sequence of an unbound or Cas9-bound guide RNA to form a PNA:RNA duplex. In some embodiments, the oligomer hybridizes to the target site by Watson-Crick binding only. Thus, in some embodiments, the oligomer does not form a PNA:DNA:DNA or PNA:DNA:PNA triplex with the DNA at the CRISPR/Cas target site. In some embodiments, the PNA is not a bis-PNA or a tail clamp PNA (tcPNA) oligomer.

In some embodiments, the nucleobase sequence can hybridize to any integer range or specific number between 1-25, 5-25, 10-25, 15-25, 1-20, 5-20, 5-15, or 8-12 nucleotides of the CRISPR RNA's (e.g. gRNA or sgRNA or crRNA, etc.) spacer sequence. In some embodiments, any subrange or specific integer percentage between 25 and 100 of the PNA oligomer hybridizes to the CRISPR RNA's spacer sequence. In some embodiments, hybridization includes 0, 1, 2, 3, 4, 5 or more mismatches, gaps, and or insertions. Thus, although the ssPNA nucleobase sequence is preferably fully complementary to the CRISPR RNA's spacer sequence, in some embodiments, the ssPNA is partially complementary. The ssPNA can be, for example, about 5 and about 50 residues in length, more preferably about 5 to about 30 residues or about 5 to about 20 residues in length, for example, 10 residues. In some embodiments, a CRISPR RNA that targets the CRISPR target site cannot bind to the protospacer sequence in the presence of the ssPNA, and preferably, a Cas enzyme that binds to the CRISPR RNA cannot bind the CRISPR/Cas target site and may have reduced binding to the CRISPR RNA.

In some embodiments, some or all of the PNA residues are modified at the gamma position. Modifications include, but are not limited to, diethylene glycol (i.e., “mini-PEG”), or substitution with one or more amino acid side chains, optionally selected from alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. In some embodiments, none of PNA residues are modified at the gamma position. In some embodiments, the heterocyclic bases are selected from uracil, thymine, cytosine, adenine, guanine, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives. The ssPNA oligomer can optionally include one or more positively charged moieties, such as lysine and/or arginine. In some embodiments, one or more positively charged moieties are present at the N-terminus, C-terminus, or both. In particular embodiments, the ssPNA includes 20 PNA residues and three lysines at each of the N-terminus and C-terminus.

The ssPNA oligomers can be packaged in a microparticle or nanoparticle. Exemplary particles include liposomes and polymeric particles.

Pharmaceutical compositions are also provided. Pharmaceutical compositions can, for example, include an effective amount of ssPNAs to reduce activity of the CRISPR/Cas at the target site. The pharmaceutical composition can further include an effective amount of the CRISPR RNA of the CRISPR/Cas system, or a nucleic acid expression construct encoding the same; an effective amount of a CRISPR RNA's that targets a similar or related CRISPR/Cas target site, or a nucleic acid expression construct encoding the same; or a combination thereof.

Two, three, four, five, or more different ssPNAs targeting different sgRNAs and thus different CRISPR/Cas target sites can also be used in combination to modulate numerous different sites at one time. For example, in some embodiments, the number of additional PNA oligomers directed to different CRISPR/Cas target sites are between 1 and 1,000 inclusive, or any subrange or specific integer number there between. The different PNAs can be in the same or different compositions and contacted with cells at the same or different times.

CRISPR/Cas gene editing compositions including the ssPNA oligomers and a spacer-sequence containing CRISPR RNA are also provided. Typically, the ssPNA is hybridized with the spacer-sequence containing CRISPR RNA. In some embodiments, the CRISPR/Cas gene editing compositions optionally also include a Cas enzyme, and in some embodiments, the CAS enzyme is complexed with the ssPNA hybridized to the CRISPR RNA. Exemplary CRISPR RNAs include a crRNA, a tracrRNA, a guide RNA (gRNA) or single guide RNA (sgRNA). Exemplary Cas enzymes include spCas9, FnCas9, SaCas9, NmCas9, St1Cas9, St3Cas9, AsCPf1, LbCpf1, and Cas13.

Pharmaceutical compositions including an effective amount of anti-spacer ssPNA oligomers, or an effective amount of CRISPR/Cas gene editing compositions including anti-spacer ssPNA oligomers to reduce activity of CRISPR/Cas at the target site are also provided.

In some embodiments, the pharmaceutical compositions include an effective amount of anti-spacer ssPNA oligomers to reduce the activity of CRISPR/Cas at the target site; and an effective amount of the CRISPR RNA, or a nucleic acid expression construct encoding the same. In some embodiments, the pharmaceutical composition includes an effective amount of anti-spacer ssPNA oligomers to reduce activity of the CRISPR/Cas at an “off-target” site of the target site. In some embodiments, the target sequence of the CRISPR RNA is 15-25 nucleotides, or any subrange of specific integers there between, and the ssPNA is 5-20 PNA units or any subrange of specific integers there between, wherein the ssPNA binds the CRISPR RNA at least 1, 2, 3, 4, 5, 6, 7, 8, 9, 10, 11, 12, 13, 14, 15, 16, 17, 18, 19, or 20 nucleotides distal to the PAM site of the spacer-sequence containing CRISPR RNA. In some embodiments, the target sequence of the gRNA is 20 nucleotides, the ssPNA is 10 PNA units and the ssPNA binds the CRISPR RNA 0, 5, 10, 15 or 20 nucleotides distal to the PAM site. In some embodiments, the ssPNA oligomer is effective to reduce or eliminate Cas localization and/or activity at the off-target site, preferably while activity at the similar or related on-target site is reduced to a lesser extent or preferably is not reduced. In some embodiments, the pharmaceutical composition further includes an effective amount of the Cas enzyme of the CRISPR/Cas, or a nucleic acid expression construct encoding the same. In exemplary embodiments, the Cas enzyme has double-strand cutting nuclease activity, for example, single strand (“nickase”) cutting activity. In other embodiments, the Cas enzyme has no strand cutting activity (i.e., is a “dead” enzyme).

In some embodiments, the composition includes an effective amount of one or more additional ssPNA oligomers directed to different CRISPR/Cas target sites. For example, in some embodiments, the number of additional ssPNA oligomers directed to different CRISPR/Cas target sites are between 1 and 1,000 inclusive, or any subrange or specific integer number there between.

Methods of reducing Cas localization and/or activity at a desired target site in the genome of a population of cells are also provided. Typically, the methods include contacting the cells with an effective amount of an anti-spacer ssPNA oligomer. In some embodiments, the ssPNA oligomer is hybridized to a spacer-sequence containing CRISPR RNA. Therefore, in some embodiments, the method includes a step of annealing the ssPNA oligomer to a spacer-sequence containing CRISPR RNA prior to contacting the cells with an effective amount of the ssPNA oligomer. The desired target site can be either an on-target site, and Cas localization and/or activity is reduced or eliminated at the desired on-target target site; and/or can be an off-target site(s), and Cas localization and/or activity is reduced or eliminated at the desired off-target target site(s).

In some embodiments, the Cas localization and/or activity is reduced or eliminated at the desired off-target target site(s) but is not eliminated at the on-target site, or Cas localization and/or activity is reduced to a lesser extent relative to the on-target site. In other embodiments, the Cas localization and/or activity is not reduced at the on-target site. In some embodiments, the desired target site includes at least 20, 30, 40, 50, 60, 70, 75, 80, 85, 90, or 95% sequence identity to another CRISPR/Cas target site, or the reverse complement thereof. In some embodiments, the desired target site includes less than 100, 95, 90, 85, 80, 75, 70, 60, 50, 40, 30, or 20% sequence identity to another CRISPR/Cas target site or the reverse complement thereof. In other embodiments, the desired target site and the CRISPR/Cas target site or its reverse complement share at least between 5-25, or any subrange or specific integer number thereof, nucleobases in common, optionally wherein the nucleobases in common are contiguous.

In some embodiments, the methods further include contacting the cells with one or more of: a gRNA that targets the desired target site, or a nucleic acid expression construct encoding the same; a Cas enzyme, or a nucleic acid expression construct encoding the same; and a donor oligonucleotide to recombine into the genome adjacent to the desired target site. Typically, the cells are contacted in vitro or ex vivo, or in vivo. In some embodiments, the Cas enzyme has double-strand cutting nuclease activity or single-strand cutting activity, and the methods include one or more steps of contacting the cells with a donor oligonucleotide, and/or editing the genome at or adjacent to the desired target site. Exemplary editing includes introducing a donor oligonucleotide and/or mutating existing nucleotides at or adjacent to the target site. An exemplary donor oligonucleotide or mutation corrects a disease-causing nucleic acid sequence. In other embodiments, the Cas enzyme does not have nuclease cutting activity. In some embodiments, the Cas enzyme is a fusion protein further including a heterologous polypeptide, such as a DNA binding domain, deaminase domain, or a protein-binding domain. Therefore, the Cas enzyme can modulate gene regulation, epigenetic editing, chromatin engineering, or imaging.

In some embodiments, the ssPNA oligomer is present at a ratio of ssPNA:sgRNA at a molar ratio of at least 1:1. In some embodiments, the ssPNA oligomer is present in an amount between 30 pmol and 250 pmol, inclusive, for example, an amount between 50 pmol and 150 pmol, inclusive. In particular embodiments, the ssPNA oligomer is present in an amount of 150 pmol.

Methods of reducing Cas localization and/or activity at a desired target site in the genome of a population of cells including contacting the cells with an effective amount of an ssPNA oligomer including a nucleic acid sequence of any one of the sequences set forth in Table 3 or Table 3A are also provided. In some embodiments, the ssPNA is annealed to a CRISPR RNA, such as a gRNA. In some embodiments, the methods include one or more steps for hybridizing the ssPNA to a gRNA prior to contacting the cells.

Compositions of CRISPR RNA/PNA duplexes are also provided. Typically, the CRISPR RNA/PNA duplexes include a CRISPR RNA having a spacer sequence that is complimentary to a protospacer sequence of a target genomic DNA, and includes from 15 to 25 contiguous nucleobases, inclusive, or any subrange of specific integers there between, and the PNA includes 5-25 contiguous nucleobases inclusive, or any subrange of specific integers there between. Typically, at least 50% up to 100% of the PNA nucleobases are complimentary to the nucleotides in the spacer sequence and at least 50%, up to 100% of the PNA nucleobases are hybridized to the complimentary nucleotides in the spacer sequence. Typically, the PNA is hybridized to the spacer sequence by Watson-Crick binding only. In some embodiments, the PNA is hybridized to 1, 2, 3, 41 5, 6, 7, 8, 9, 10, 11, 12, 13 14, 15, 16, 17, 18, 19, 20, 21, 22, 23, 24 or 25 nucleotides of the CRISPR RNA spacer sequence. The hybridization can include 0, 1, 2, 3, 4, 5 or more mismatches, gaps, and/or insertions. In particular embodiments, the PNA is hybridized to all the nucleotides in the spacer sequence. Typically, the PNA is or includes 10 or 20 nucleic acids in length.

In some embodiments, the CRISPR RNA/PNA duplex exhibits reduced binding to the protospacer sequence, and optionally cannot bind to the protospacer sequence as compared to a corresponding CRISPR RNA in the absence of the PNA. Typically, the PNA includes at least an integer number between 1 and 25, inclusive, of bases between −20 and −1 of the target site's protospacer sequence, wherein −1 is the last nucleobase of the protospacer and +1 is the first nucleobase of the PAM sequence. In particular embodiments, the PNA within the CRISPR RNA/PNA duplex includes a nucleic acid sequence of any one of the sequences set forth in Table 3 or Table 3A.

Pharmaceutical compositions including an effective amount of a CRISPR RNA/PNA duplex to reduce activity of the CRISPR/Cas at the target site are also provided.

Methods of reducing Cas localization and/or activity at a desired target site in the genome of a population of cells including contacting the cells with an effective amount of a CRISPR RNA/PNA duplex, or the pharmaceutical composition thereof are also provided. In some embodiments, the desired target site is an on-target site, and Cas localization and/or activity is reduced or eliminated at the desired on-target target site. In other embodiments, the desired target site is an off-target site, and Cas localization and/or activity is reduced or eliminated at the desired off-target target site.

In some embodiments, the Cas localization and/or activity is reduced or eliminated at the desired off-target target site, but is not eliminated at the on-target site. In some embodiments, Cas localization and/or activity is reduced to a lesser extent relative to the on-target site, or Cas localization and/or activity is not reduced at the on-target site. In some embodiments, the desired target site includes at least 20, 30, 40, 50, 60, 70, 75, 80, 85, 90, or 95% sequence identity to another CRISPR/Cas target site or the reverse complement thereof. In other embodiments, the desired target site includes less than 100, 95, 90, 85, 80, 75, 70, 60, 50, 40, 30, or 20% sequence identity to another CRISPR/Cas target site or the reverse complement thereof. In some embodiments, the desired target site and the other CRISPR/Cas target site or its reverse complement share at least between 5-25, or any subrange or specific integer number thereof, nucleobases in common, optionally wherein the nucleobases in common are contiguous.

In some embodiments, the methods further include contacting the cells with one or more of: a CRISPR RNA that targets the desired target site, or a nucleic acid expression construct encoding the same; a Cas enzyme, or a nucleic acid expression construct encoding the same; or a donor oligonucleotide to recombine into the genome adjacent to the desired target site. The cells can be contacted in vitro or ex vivo, or in vivo.

In some embodiments, the Cas enzyme has double-strand cutting nuclease activity or single-strand cutting activity, and the methods optionally further include contacting the cells with a donor oligonucleotide. Therefore, in some embodiments, the methods edit the genome at or adjacent to the desired target site. Exemplary editing includes introducing a donor oligonucleotide and/or mutating existing nucleotides at or adjacent to the target site. An exemplary the donor oligonucleotide or mutation corrects a disease-causing nucleic acid sequence.

In some embodiments, the Cas enzyme does not have nuclease cutting activity. In some embodiments, the Cas enzyme is a fusion protein further including a heterologous polypeptide. Exemplary heterologous polypeptides include a DNA binding domain, deaminase domain, or a protein-binding domain. In some embodiments, the Cas enzyme modulates gene regulation, epigenetic editing, chromatin engineering, or imaging.

Methods of reducing Cas localization and/or activity at a desired target site in the genome of a population of cells are provided. The methods typically include contacting the cells with an effective amount of the ssPNA oligomer.

In some embodiments, the desired target site is a genomic locus as the CRISPR/Cas target site to which the ssPNA hybridizes (i.e., the ssPNA binds to an sgRNA that targets the desired target site). This strategy allows for “on-target” modulation of CRISPR/Cas, and can be used to, for example, modulate the time and extent of on-target Cas enzyme localization and activity. In some embodiments, Cas localization and/or activity is reduced or eliminated at the desired target site.

In some embodiments, the desired target site is a different genomic locus from the CRISPR/Cas target site to which the ssPNA can hybridize (i.e., the ssPNA binds to an sgRNA that targets an off-target site). This strategy allows for “off-target” modulation of CRISPR/Cas. In some embodiments, Cas localization and/or activity is reduced or eliminated at the CRISPR/Cas target site (i.e., the “off-target” site), preferably while Cas localization and/or activity at the desired target site (i.e., the “on-target” site) is reduced to a lesser extent or is not reduced relative to the CRISPR/Cas target site. In some embodiments, the desired target site has at least 20, 30, 40, 50, 60, 70, 75, 80, 85, 90, or 95% sequence identity to the CRISPR/Cas target site or the reverse complement thereof. In some embodiments, the desired target site has less than 100, 95, 90, 85, 80, 75, 70, 60, 50, 40, 30, or 20% sequence identity to the CRISPR/Cas target site or the reverse complement thereof. In some embodiments, the desired target site and the CRISPR/Cas target site or its reverse complement share at least between 5-25, or any subrange or specific integer number thereof, nucleobases in common, optionally wherein the nucleobases in common are contiguous.

The methods typically also include contacting the cells with a CRISPR RNA that targets the desired target site, or a nucleic acid expression construct encoding the same, as well as a Cas enzyme, or a nucleic acid expression construct encoding the same. In some embodiments, the cells are also contacted with a donor oligonucleotide to be recombined into the genome adjacent to the desired target site. The cells can be contacted in vitro, ex vivo, or in vivo.

The Cas enzyme can be any Cas enzyme. For example, Cas enzyme can have double-strand cutting nuclease activity or single-strand cutting activity, single strand cutting nuclease activity, or no nuclease activity. The Cas enzyme can be a fusion protein further having a heterologous polypeptide domain such as a transcription activator or repressor, chromatin modifier, deaminase domain, etc. Thus, the disclosed methods can be used not only for genome modification, but also to modulate gene regulation, epigenetic editing, chromatin engineering, or imaging.

### BRIEF DESCRIPTION OF THE DRAWINGS

FIG. 1A is an illustration showing how CRISPR/Cas binds to and modifies target DNA, and how ssPNA can modulate this interaction. Under normal circumstances (bottom) Cas9 uses the spacer sequence of the internal sgRNA to identify and base pair dsDNA sequences, followed by coordinated dsDNA cleavage. In the presence of sequence-matched ssPNA (top), a stable PNA:RNA duplex forms with the spacer sequence of the sgRNA. As a result, Cas9 is unable to coordinate its target sequence and no cleavage or coordination occurs. FIG. 1B is a graph showing Normalized OD (0.95-1.35) over temperature (20-80° C.) for each of DNA:RNA, RNA:RNA, and PNA:RNA, respectively.

FIGS. 2A-2B are images of electrophoretic gels showing the results of an assay designed to investigate the sequence specificity of PNA binding to sgRNA and Cas9 ribonucleoprotein (RNP) complexes. FIG. 2C is a bar graph showing Integrated Density (0-25 a.u.) over TAMRA PNA dose (0-20 pmol) for each of BFP Cas9 RNP and HBB Cas9 RNP, respectively. FIG. 2D is a bar graphing showing the Percent Editing (%) of antispacer PNA, antispacer DNA, and GFP Non-targeting PNA oligonucleotides, at various doses as indicated.

FIGS. 3A and 3B are bar graphs showing Percent Editing (%) of BFP- and HBB-targeting PNAs at the BFP (3A) and HBB (3B) target sites. FIGS. 3C and 3D are bar graphs showing Percent Editing (%) for each of over PNA Dose (0-250 pmol) for each of BFP Antispacer PNA and HBB Antispacer PNA (FIG. 3C), and Indel or HDR, at 0, 2, 4, 6, 8, 10, 12, or 24 hours after Cas9 RNP Nucleofection, as well as for Cas9/ssODN only (FIG. 3D).

FIG. 4A is a bar graph of percent editing for Cas9 RNP-nucleofected cells pre-treated with PNAs (250 pmol) designed to bind either DNA strand across the BFP Cas9 binding site, showing the percent Editing (%) when PNA are designed to target various stretch of the target locus relative to the PNA site utilizing a Cis-Spacer (top bar) or bottom Anti-Spacer (bottom bar). PNA binding sites are described relative to the PAM/protospacer junction (0 bp) and are theoretically oriented to bind the target strand (homologous to gRNA, or “cis-spacer”) or the nontarget strand (complementary to gRNA, or “anti-spacer”). −20 to −1 and −10 to +9 antispacer PNAs are the only molecules with complementarity to the PAM-proximal gRNA spacer sequence. Top bar represents Cas9 only treated control. Bars represent mean±s.d. from n=3 independent experiments.

FIG. 4B is a bar graph of the effect of ssPNA sequence, length, and relative contributions of spacer sequence segments for inhibition when bound by PNA on modulation of Cas9 activity, showing the Percent Editing (%) over PNA Doses of 0, 50, 100, 150, 200 or 250 pmol, respectively, when PNA are designed to target various stretch of the target locus relative to the PNA site utilizing either an Antispacer 20mer PNA; PAM-proximal 10mer PNA; PAM-middle 10mer PNA, or PAM-distal 10mer PNA, respectively. FIG. 4C is a graph of Percent Editing (%) for HBB PAM-proximal PNA at doses of 0, 50, 100 or 150 pmol, respectively. The fold-difference between % editing for each dosage is indicated.

FIG. 5 is a bar graph showing Percent Editing (%) as a function of time elapsed after Cas9 RNP nucleofection, as indicated.

FIG. 6 is a line graph showing Fold-Difference (ΔΔCt) of POU5F1 expression overtime in K562 cells, K562 cells expressing CRISPRa (dCas9-VPR) and treated with NT PNA, and K562 cells expressing CRISPRa and treated with POU5F1 PNA).

FIG. 7A is an illustration of a 10mer PAM-distal antispacer PNA, such as those utilized in Example 6 (and FIGS. 7B and 7C) bound to a target gRNA. FIG. 7B is a schematic depicting the failure of Cas9-sgRNA to seed R-loop, due to potent inhibition. FIG. 7C is a schematic depicting the initiation of R-loop formation by Cas9-sgRNA bound to a 10mer PAM-distal antispacer PNA, and subsequent displacement of the PNA.

FIGS. 8A to 8M are graphs showing Percent Editing (%) over PNA dose (0-150 pmol) for each of VEGFA ON (FIG. 8A); VEGFA OT2 (FIG. 8B); VEGFA OT4 (FIG. 8C); ZSCAN ON (FIG. 8D); ZSCAN OFF (FIG. 8E); CCR5 ON (FIG. 8F); CCR5 OFF (FIG. 8G); EMX1 ON (FIG. 8H); EMX1 OT1 (FIG. 8I); FANCF ON-anneal (FIG. 8J); FANCF OT1-anneal (FIG. 8K); EMX1 ON-anneal (FIG. 8L); EMX1 OT1-anneal (FIG. 8M), respectively. FIGS. 8N and 8O are graphs showing Percent Editing (%) at FANCF ON (FIG. 8N) and FANCF OT1-target (FIG. 8O) sites over PNA doses of 0, 50, 100 or 150 pmol, in cells nucleofected with vehicle (water) or 250 pmol of a 20mer PNA targeting part of the protospacer (6nt), PAM, and upstream sequence (I1nt) of the FANCF off-target binding site (FANCF OT1). The nucleic acid sequences GCTGCAGAAGGGATTCCATG (SEQ ID NO:19) and GCTGCAGAAGGGATTCCAAG (SEQ ID NO:69) are depicted in FIGS. 8N and 8O, respectively.

FIGS. 9A-9D are graphs showing Percent Editing (%) over PNA dose (0 or 50 pmol) for each of EMX1 ON-HEK293 (FIG. 9A); EMX1 OT1-HEK293 (FIG. 9B); EMX1 ON-U2OS (FIG. 9C); and EMX1 OT1-U2OS (FIG. 9D), respectively. FIGS. 9E-9H are graphs showing Percent Editing (%) over PNA dose (Cas9 or Cas9-PNA) for each of FANCF ON-HEK293 (FIG. 9E); FANCF OT1-HEK293 (FIG. 9F); FANCF ON-U2OS (FIG. 9G); FANCF OT1-U2OS (FIG. 9H), respectively. FIG. 9I is a graph plotting percent reduction in on-target editing for PAM-distal 10mer PNA 50 pmol conditions as a function of calculated Doench on-target affinity score, showing the precent reduction in on-target editing (calculated as: 100−(% editing(50 pmol)/mean % editing(0 pmol)*100) graphed as a function of predicted on-target affinity score for each of CCR5, ZSCAN2, VEGFA, FANCF and BFP, respectively. Best-fit line was determined by simple linear regression model (Y=−1.568X+104.6) and dashed lines represent 95% CI. Calculated R2 value (0.853), P value (<0.0001), and corresponding gene targets are labeled on plot. FIG. 9J is a graph of percent off-target editing reduction for 10mer PAM-distal PNA-treated conditions (50 pmol) as a function of mean percent baseline editing, showing the precent reduction in off-target editing graphed as a function of percent baseline editing (%) for each of CCR5 (CCR2 OT), ZSCAN2 OT1, VEGFA OT4, VEGFA OT2, FANCF OT1 and EMX OT1, respectively. Best-fit line was determined by simple linear regression model (Y=−3.659X+75.14) and dashed lines represent 95% CI. Calculated R2 value (0.337), P value (0.0116), and corresponding gene targets are labeled on plot. FIG. 9K is a graph showing the precent reduction in on-target editing (calculated as: 100−(% editing(50 pmol)/mean % editing(0 pmol)*100) graphed as a function of Baseline editing (%) for each of CCR5, ZSCAN2, VEGFA, FANCF and BFP, respectively.

FIGS. 10A-10D are bar graphs. FIG. 10A shows Specificity Ratio (0-400) for gRNA and 50 pmol PNA, respectively, for each of FANCF, EMX1, VEGFA, ZSCAN2, and CCR5, respectively. FIG. 10B shows Specificity Ratio (0-32) for Cas9 only and 50 pmol PNA, respectively, for each of EMX1 and FANCF in each of K562, HEK293, and U2OS cells, respectively. FIG. 10C shows Specificity Ratio (0-128) for each of Cas9 only, Pre-treat and Annealed samples, respectively, for each of EMX1 and FANCF, respectively. FIG. 10D shows Specificity Ratio (0-80) for each of Cas9 only, and Cas9 treated with PNA (+PNA), respectively, for each of EMX1 and FANCF, respectively.

FIGS. 11A-11B are graphs illustrating the results of assay designed to test if antispacer PNAs can modulate acetyltransferase-fused (dCas9-p300) epigenetic editing. FIG. 11A shows Relative MYOD1 mRNA over Time (0-192 hrs) for each of BFP Antispacer PNA and POU5F1 Antispacer PNA, respectively. FIG. 11B shows Relative MYOD1 mRNA over Time (0-240 hrs) for each of BFP Antispacer PNA and MYOD1 Antispacer PNA, respectively. FIG. 11C is a bar graph of percent occupancy over (calculated by percent input method with 2% input) at selected timepoints relative to PNA nucleofection (250 pmol dose). Parent K562 (no dCas9-p300) and stable K562-dCas9-p300 (No PNA) levels prior to nucleofection are labelled.

FIG. 12 is a bar graph of percent cell viability relative to mock nucleofected control (0 pmol) for Cas9 RNP, selected PNAs, and ssDNA across treatment doses, showing Viability (% relative to mock Tx) over dose (0, 50, 100, 150, 200, or 250 pmol, respectively) for each of Cas9 RNP, BFP 20mer PNA, HBB 20mer PNA, MYOD 20mer PNA, BFP PAM-Proximal 10mer, FANCF PAM-Distal 10mer, and BFP 20mer ssDNA, respectively, as indicated. K562 cells were treated with specified doses and allowed 72 hours to recover. Viability was calculated relative to 0 pmol (mock) conditions for each experiment and measured using CellTiter-Glo 2.0 Luminescent Cell Viability Assay (Promega). Points represent mean±s.d. from n=4 independent experiments.

### DETAILED DESCRIPTION OF THE INVENTION

### I. Definitions

As used herein, the term “modified nucleotide” or “chemically modified nucleotide” defines a nucleotide that has a chemical modification of one or more of the heterocyclic base, sugar moiety or phosphate moiety constituents.

As used herein, the term “recombinogenic” refers to a DNA fragment, oligonucleotide, peptide nucleic acid, or composition as being able to recombine into a target site or sequence or induce recombination of another DNA fragment, oligonucleotide, or composition.

As used herein, the terms “protospacer sequence”, “protospacer” and “priming protospacer” are used interchangeably to refer to a genomic nucleic acid sequence that is complimentary to, and therefore specifically recognized by a CRISPR RNA, such as a guide RNA (gRNA) that directs the specific nuclease activity of a Cas enzyme to a target nucleic acid sequence.

As used herein, the term “protospacer adjacent motif”, or “PAM” or “PAM sequence” refer to a short DNA sequence (usually 2-6 base pairs in length) that follows the genomic DNA region targeted for cleavage by the CRISPR system, such as CRISPR-Cas9. The PAM is required for a Cas nuclease to cut and is generally found 3-4 nucleotides downstream from the Cas nuclease cut site.

As used herein, the term “spacer sequence”, or “spacer” refers to a nucleic acid motif that is complimentary to and therefore forms Watson-Crick base pairing interactions with a corresponding protospacer sequence in the genomic DNA of an organism. For example, a CRISPR RNA typically includes a spacer sequence that drives specific interaction with a corresponding protospacer sequence within genomic DNA.

As used herein, the term “eukaryote” or “eukaryotic” refers to organisms or cells or tissues derived therefrom belonging to the phylogenetic domain Eukarya such as animals (e.g., mammals, insects, reptiles, and birds), ciliates, plants (e.g., monocots, dicots, and algae), fungi, yeasts, flagellates, microsporidia, and protists.

As used herein, the term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a termination sequence. The construct may also include selectable marker gene(s) and other regulatory elements for expression.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

As used herein, the term “vector” refers to a replicon, such as a plasmid, phage, or cosmid, into which another DNA segment may be inserted so as to bring about the replication of the inserted segment. The vectors can be expression vectors.

As used herein, the term “expression vector” refers to a vector that includes one or more expression control sequences.

As used herein, the term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

As used herein, the terms “transformed,” “transgenic,” “transfected” and “recombinant” refer to a host organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

As used herein, the term “endogenous” with regard to a nucleic acid refers to nucleic acids normally present in the host.

As used here, the term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompass “exogenous” and “non-native” elements.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, the term “pharmaceutically acceptable” refers to a material that is not biologically or otherwise undesirable, i.e., the material may be administered to a subject without causing any undesirable biological effects or interacting in a deleterious manner with any of the other components of the pharmaceutical composition in which it is contained.

As used herein, the term “carrier” or “excipient” refers to an organic or inorganic ingredient, natural or synthetic inactive ingredient in a formulation, with which one or more active ingredients are combined. The carrier or excipient would naturally be selected to minimize any degradation of the active ingredient and to minimize any adverse side effects in the subject, as would be well known to one of skill in the art.

As used herein, the term “treat” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “inhibit” or “reduce” means to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, a “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from a nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid sequence, and then expressing the nucleic acid in an appropriate host cell under conditions in which the fusion protein is produced.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. The use of any and all examples, or exemplary language (e.g., “such as”) provided herein, is intended merely to better illuminate the description and does not pose a limitation on the scope of the description unless otherwise claimed.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed. All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Components

Provided herein are sequence-specific synthetic PNA oligos that can hybridize to the spacer sequence of a CRISPR RNA and prevent Cas enzymes from localizing to dsDNA and eliciting an effect. Also provided are ribonucleic acid-peptide nucleic acid complexes formed between CRISPR RNA and a corresponding sequence-specific PNA hybridized to part or all of the spacer sequence of the CRISPR RNA such as an sgRNA, also referred to as pre-annealed complexes. In some embodiments, the CRISPR RNA-PNA complexe further includes a Cas protein.

The disclosed compositions address the need to control the timing, duration, efficiency, and specificity of CRISPR-Cas systems and their wide-ranging applications. Such a system has the capacity to improve control and precision of Cas enzyme targeting to expand applications and safety in research and therapeutic contexts.

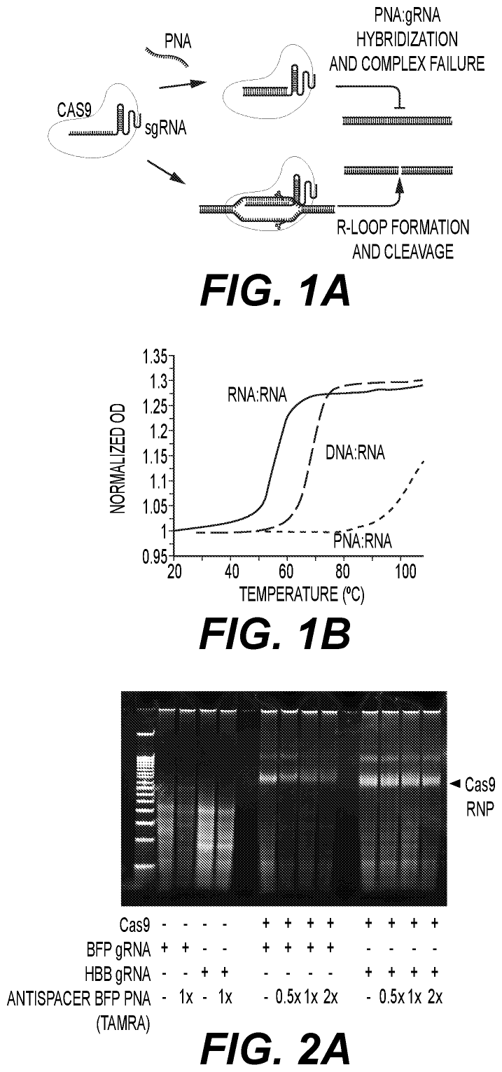

FIG. 1A summarizes an approach to improve control and precision of Cas enzyme targeting. PNAs are DNA mimics with charge neutral polyamide backbones. Compared to DNA, PNAs have reduced repulsion between backbones when hybridized in a sequence specific fashion to DNA. As a result, PNAs form high affinity duplex structures with complementary DNA and RNA sequences. Using Cas9 as an example of a Cas enzyme, FIG. 1A illustrates how it recognizes its cognate match in the genome when a stretch of dsDNA sequence matches a complementary 20 nucleotide sequence encoded in its guide RNA “protospacer” sequence and only when in proximity to an “NGG” PAM motif. Cas9 recognizes its match sequence exclusively in the context of double stranded DNA. A ssPNA molecule matching the CRISPR RNA spacer sequence can anneal with the CRISPR RNA and reduce ribonucleoprotein complex binding to the DNA duplex target site and impart nuclease activity or other non-enzymatic activity mediated by the Cas protein. Each of these elements, and use thereof are discussed in more detail below. Therefore, ssPNA molecules that interact with CRISPR RNA sequences that include a spacer sequence and minimize or reduce off-target interactions are provided. Typically the PNAs are single stranded PNA oligomers having one or more amino acid residues flanking a nucleoside sequence configured to bind to a protospacer. The length of the nucleoside sequence is typically from 10 to 30 bases. The nucleoside sequence of the ssPNA is typically configured to bind to the CRISPR RNA spacer sequence at a site that is at a specified location relative to the protospacer adjacent motif (PAM). Therefore, in some embodiments, the nucleoside sequence of the ssPNA is a 10 mer configured to bind a 20 nucleoside CRISPR RNA spacer sequence at a site that is distal to the PAM (i.e., at nucleoside positions 10-20). In other embodiments, the nucleoside sequence of the ssPNA is a 10 mer configured to bind a 20 nucleoside CRISPR RNA spacer sequence at a site that is middle to the PAM (i.e., at nucleoside positions 5-15). In other embodiments, the nucleoside sequence of the ssPNA is a 10 mer configured to bind a 20 nucleoside CRISPR RNA spacer sequence at a site that is proximal to the PAM (i.e., at nucleoside positions 0-10).

### A. Peptide Nucleic Acids

Peptide nucleic acid oligomers (PNAs) and design parameters for use in spatiotemporal modulation of CRISPR/Cas technology are provided. Peptide nucleic acids are molecules in which the phosphate backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that are similar to oligonucleotides, but are achiral and neutrally charged molecules. Peptide nucleic acids are composed of peptide nucleic acid monomers (also referred to herein as residues). The heterocyclic bases can be any of the standard bases (“B”, e.g., uracil, thymine, cytosine, adenine and guanine) or a modified base such as inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives.

PNAs can bind to DNA via Watson-Crick hydrogen bonds, but with binding affinities significantly higher than those of a corresponding nucleotide composed of DNA or RNA. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

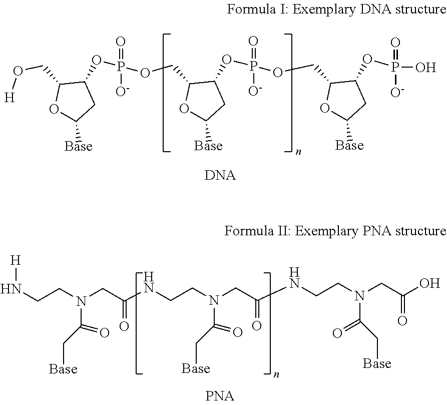

PNA oligomer sequences are generally presented in N-terminal-to-C-terminal orientation. In some embodiments, PNA oligomer sequences can be presented in the form: NH₂— “nucleobase sequence” —OH orientation, wherein the NH₂ represents the N-terminal amine group of an unmodified PNA oligomer and the —OH represents the hydroxyl component of the C-terminal carboxyl group of the polymer.

Common modifications to PNA are discussed in Sugiyama and Kittaka, Molecules, 18:287-310 (2013)) and Sahu, et al., J. Org. Chem., 76, 5614-5627 (2011), each of which are specifically incorporated by reference in their entireties, and include, but are not limited to, incorporation of charged amino acid residues, such as lysine at the termini or in the interior part of the oligomer; inclusion of polar groups in the backbone, carboxymethylene bridge, and in the nucleobases; chiral PNAs bearing substituents on the original N-(2-aminoethyl)glycine backbone; replacement of the original aminoethylglycyl backbone skeleton with a negatively-charged scaffold; conjugation of high molecular weight polyethylene glycol (PEG) to one or both of the termini; fusion of PNA to DNA to generate a chimeric oligomer, redesign of the backbone architecture, conjugation of PNA to DNA or RNA. These modifications improve solubility but often result in reduced binding affinity and/or sequence specificity.

In preferred embodiments, PNAs include positively charged moieties to increase the solubility of the PNA and increase the affinity of the PNA for duplex DNA. Commonly used positively charged moieties include the amino acids lysine and arginine, although other positively charged moieties may also be useful. Lysine and arginine residues are most typically added to the carboxy and/or the N-terminus of a PNA strand.

In some embodiments, the some or all of the PNA monomers are modified at the gamma position in the polyamide backbone (γPNAs) as illustrated below (wherein “B” is a nucleobase and “R” is a substitution at the gamma position).

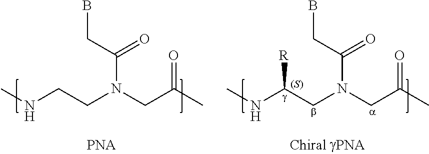

Substitution at the gamma position creates chirality and provides helical pre-organization to the PNA oligomer, yielding substantially increased binding affinity to the target DNA (Rapireddy, et al., Biochemistry, 50(19):3913-8 (2011)). Other advantageous properties can be conferred depending on the chemical nature of the specific substitution at the gamma position (the “R” group in the chiral γPNA above).

One class of γ substitution, is miniPEG, but other residues and side chains can be used, and even mixed substitutions can be used to tune the properties of the oligomers. “MiniPEG” and “MP” refers to diethylene glycol. MiniPEG-containing γPNAs are conformationally preorganized PNAs that exhibit superior hybridization properties and water solubility as compared to the original PNA design and other chiral γPNAs. γPNAs prepared from L-amino acids adopt a right-handed helix, while those prepared from D-amino acids adopt a left-handed helix; however, only the right-handed helical γPNAs hybridize to DNA or RNA with high affinity and sequence selectivity. In the most preferred embodiments, some or all of the PNA monomers are miniPEG-containing γPNAs (Sahu, et al., J. Org. Chem., 76, 5614-5627 (2011). In some embodiments, PNA oligomers are prepared wherein every other PNA monomer is a miniPEG-containing γPNA.

In some embodiments the PNA include additional or alternative y substitutions or other PNA chemical modifications including but limited to those introduced above and below. Examples of γ substitution with other side chains include that of alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. The “derivatives thereof” herein are defined as those chemical moieties that are covalently attached to these amino acid side chains, for instance, to that of serine, cysteine, threonine, tyrosine, aspartic acid, glutamic acid, asparagine, glutamine, lysine, and arginine.

The PNA can also be modified to include a guanidine-G-clamp (“G-clamp”) PNA monomer(s) to enhance PNA binding. PNAs with substitution of cytosine by clamp-G (9-(2-guanidinoethoxy) phenoxazine), a cytosine analog that can form five H-bonds with guanine, and can also provide extra base stacking due to the expanded phenoxazine ring system and substantially increased binding affinity. In vitro studies indicate that a single clamp-G substitution for C can substantially enhance the binding of a PNA-DNA duplex by 23oC (Kuhn, et al., Artificial DNA, PNA & XNA, 1(1):45-53(2010)).

The structure of a clamp-G monomer-to-G base pair (clamp-G indicated by the “X”) is illustrated below in comparison to C-G base pair.

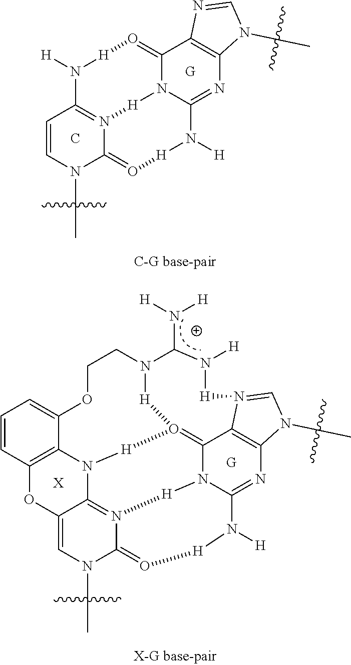

Some studies have shown improvements using D-amino acids in peptide synthesis.

### 1. PNA Oligomer Target Sequence Considerations

Preferably, the disclosed PNA oligomers are typically single-stranded PNA oligomers, and thus are also referred to herein as ssPNA or ssPNAs. They are typically between about 5 and about 50 residues in length or any integer range or specific integer number thereof, more preferably about 10 to about 30 residues or about 15 to about 25 residues in length. In the Examples below, the PNA oligomers used were 10 or 20 residues in length.

The disclosed PNA oligomers bind the spacer sequence of a CRISPR RNA. The spacer sequence of the CRISPR RNA is designed to bind to a predetermined target region referred to herein as the “target sequence,” “target region,” or “target site.” As discussed in Example 1 below, typically, the Cas9 enzyme recognizes its cognate match in the genome when a stretch of dsDNA “protospacer” sequence matches a complementary 20 nucleotide sequence encoded in its guide RNA “spacer” sequence and only when in proximity to an “NGG” PAM motif. Cas9 recognizes its match sequence exclusively in the context of double stranded DNA. Typically, the disclosed PNA oligomers are designed to bind to the spacer sequence of an sgRNA, and thus can be the same or similar to the target DNA's protospacer sequence. Thus, the target sequence can be, e.g., the CRISPR RNA (e.g., sgRNA) spacer sequence and is homologous to the genomic DNA protospacer sequence targeted by the sgRNA.

Typically, an ssPNA molecule matching the target sequence will bind to the CRISPR RNA to create a new PNA:RNA duplex. This interaction is believed to obstruct Cas proteins from identifying and/or complexing with its target DNA substrate and may also reduce binding of the Cas proteins from binding to CRISPR RNA. Thus, the enzyme will fail to bind or impart any nuclease activity or other activities, e.g., localization of heterologous proteins that can be imparted by non-enzymatic Cas proteins.

Thus, the disclosed PNA oligomers are typically designed to hybridize with a desired sgRNA. Preferably, the PNA hybridizes with the sgRNA target site by Watson-Crick binding, optionally by Watson-Crick binding alone. Thus, in some embodiments, the PNA oligomer does not form a triplex with the target site, either by binding to the DNA duplex (i.e., PNA:DNA:DNA triplex, without DNA strand displacement), or by a combination of Watson-Crick and Hoogsteen binding (i.e., PNA:DNA:PNA triplex with DNA strand displacement). In some embodiments, the PNA is not a bis-PNA or a tail clamp PNA (tcPNA).

The sequence of the PNA is directly tied to the spacer sequence of the CRISPR gRNA. The PNA should be capable of hybridizing to the RNA's spacer sequence and reducing or preventing CRISPR RNA binding to the genomic target site, and thus also Cas enzyme localization, to the target site. This sequence can also be referred to as an anti-spacer sequence

PNAs should be designed to cover part or all of the spacer sequence. In some examples, the PNAs are designed to specifically cover at least any integer number between 1-25, 5-25, 10-25, 15-25, 1-20, 5-20, 5-15, or 8-12 nucleotides of the CRISPR RNA (e.g. gRNA or sgRNA or crRNA) spacer sequence.

The sequence of the PNA oligomer can be any sequence, provided it is designed according to the disclosed guidelines and reduces CRISPR/Cas activity. Because the CRISPR RNA's spacer sequence binds to the DNA's protospacer sequence, the ssPNA can be the same or similar to the protospacer target strand sequence, and this can be a sequence that is within the coding DNA sequence of the target gene or within an intron. The sequence can also be within DNA sequences which regulate expression of the target gene, including promoter or enhancer sequences or sites that regulate RNA splicing. The target can also be an intergenic region of interest.

The PNA is typically substantially or fully complementary to a guide RNA spacer sequence that specifies the DNA target sequence. There are a variety of structural motifs available which can be used to determine the nucleotide sequence of a substantially complementary oligonucleotide. Preferably, the PNA binds to or hybridizes to the target sequence under conditions of high stringency and specificity. Reaction conditions for in vitro hybridization to a target nucleic acid sequence may vary, depending on factors such as the length of the molecules, the number of G:C and A:T base pairs, and the composition of the buffer utilized in the hybridization reaction. In some embodiments, 1, 2, 3, 4, 5 or more mismatches, gaps, and or insertions are tolerated.

When considered in the context of the PAM, the ssPNA can be described as binding “distal” (i.e., far), “proximal” (i.e., near) or “middle” (i.e., between distal and proximal) with respect to the location of the protospacer adjacent motif (PAM) that is associated with the protospacer sequence that is the target of the CRISPR RNA (e.g. gRNA or sgRNA or crRNA). For example, in some embodiments, ssPNAs are designed to be smaller than the entire spacer sequence, and to specifically cover a portion of the spacer sequence that includes residues any number between 1-25, 5-25, 10-25, 10-20, 1-10, 15-25, 1-20, 5-20, 5-15, or 8-12 nucleotides away from the location of the PAM of the corresponding target sequence. In particular embodiments, an ssPNA is smaller than the spacer sequence of a CRISPR RNA, such as a gRNA, and therefore anneals to cover only a portion of the spacer sequence. For example, in some embodiments, binding of the ssPNA covers less than 100% of the nucleosides in the spacer sequence. Therefore, in some embodiments, binding of the ssPNA covers approximately 10%, 20%, 30%, 40%, 50%, 60%, 70%, 80%, or 90%, of the nucleosides in the spacer sequence, or more than 90%, but less than 100% of the nucleosides in the spacer sequence. In some embodiments, an ssPNA is measured in PNA units. For example, in some embodiments, an ssPNA unit is equivalent to one nucleoside with respect to binding and covering of the nucleoside. In some embodiments, an ssPNA is complimentary to less than 100% of the nucleosides of a spacer sequence of a CRISPR RNA, for example, an ssPNA is complimentary to less than 10%, 20%, 30%, 40%, 50%, 60%, 70%, 80%, or 90%, of the nucleosides in the spacer sequence. When an ssPNA covers less than 100% of the nucleosides of a spacer sequence to which it binds, and/or is complimentary to less than 100% of the nucleosides in the spacer sequence to which it binds, the ssPNA is considered as binding to a portion of the spacer sequence. The portion of the spacer to which the ssPNA binds is identified according to the location of the PAM in the corresponding protospacer sequence in the genomic DNA. In an exemplary embodiment, an ssPNA that is 10 PNA units in size binds to 100% of the complimentary nucleosides in a spacer sequence of 20 contiguous nucleosides that are numbered from −1 to −20 relative to the location of the PAM in the corresponding protospacer sequence in the genomic DNA; if the ssPNA binds to nucleosides at positions −1 to −10, it is considered to be binding “proximal” to the PAM; if the ssPNA binds to nucleosides at positions −5 to −15, it is considered to be binding “middle” to the PAM; and if the ssPNA binds to nucleosides at positions −10 to −20, it is considered to be binding “distal” to the PAM.

### B. CRISPR/Cas Technology

The disclosed anti-CRISPR PNA compositions and methods are typically used in conjunction with a CRISPR/Cas technology. The technologies include those element or elements that can be used to induce single or a double strand break in the target cell's genome, as well as those that do not induce any strand break (i.e., catalytically inactive Cas proteins). Exemplary components of CRISPR/Cas technology are discussed in more detail below, and each can be deployed as a mature elements, or can be deployed by vectors, e.g., plasmids, viruses, etc., that encode the mature elements. Thus, CRISPR/Cas technology can be introduced into cells of a interest by transfection of nucleic acids encoding the elements, as the elements themselves (e.g., a ribo-protein complex), or a combination thereof (e.g., a nucleic acid encoding a cas gene and an sgRNA, etc.). Thus, even where not explicitly stated below, each of these means of deploying CRISPR/Cas technology, are expressly provided for all technologies, applications, and methods.

The disclosed anti-CRISPR PNA compositions and methods can be used in combination with any CRISPR/Cas-based technology or methodology. The uses of CRISPR/Cas technology are diverse, and are not limited to gene-editing tool. The application areas also include gene regulation, epigenetic editing, chromatin engineering, and imaging. See, e.g., Adli, et al., “The CRISPR tool kit for genome editing and beyond,” Nat Commun., 9(1):1911. doi: 10.1038/s41467-018-04252-2 (2018), which is specifically incorporated by reference herein in its entirety.

### 1. CRISPR/Cas Elements

CRISPR (Clustered Regularly Interspaced Short Palindromic Repeats) is an acronym for DNA loci that contain multiple, short, direct repetitions of base sequences. CRISPRs are often associated with cas genes which code for proteins that perform various functions related to CRISPRs. The CRISPR/Cas system functions as a prokaryotic immune system by conferring resistance to exogenous genetic elements such as plasmids and phages thereby imparting a form of acquired immunity. Endogenous CRISPR spacers recognize and silence exogenous genetic elements in a manner similar to RNAi in eukaryotic organisms.

As used herein, CRISPR/Cas-mediated genome editing composition refers to the elements of a CRISPR system needed to carry out CRISPR/Cas-mediated genome editing in a mammalian subject. As discussed in more detail below, CRISPR/Cas-mediated genome editing compositions typically include one or more CRISPR RNAs: crRNA, a tracrRNA (or chimeric thereof also referred to a guide RNA or single guide RNA), and a Cas enzyme. The CRISPR/Cas-mediated genome editing composition can optionally include a donor polynucleotide that can be recombined into the target cell's genome at or adjacent to the target site (e.g., the site of single or double stand break induced by the Cas enzyme, e.g., Cas9).

The CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By a Cas enzyme and designed CRISPRs into a cell, the organism's genome can be cut and modified at any desired location. Methods of preparing compositions for use in genome editing using the CRISPR/Cas systems are described in detail in WO 2013/176772 and WO 2014/018423, which are specifically incorporated by reference herein in their entireties.

In general, “CRISPR system” refers collectively to transcripts and/or other elements involved in the expression of or directing the activity of CRISPR-associated (“Cas”) genes, including the Cas enzyme itself, or a nucleic acid encoding it; a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or a nucleic acid(s) encoding the foregoing, and other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

a. Guide RNAs

As discussed in more detail below, in some embodiments, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct is also referred to herein as a guide RNA or gRNA (or single-guide RNA (sgRNA)). Within an sgRNA, the crRNA portion can be identified as the ‘target sequence’ and the tracrRNA is often referred to as the ‘scaffold’.

In some embodiments, one or more elements of a CRISPR system is derived from a type I, type II, or type III CRISPR system. In some embodiments, one or more elements of a CRISPR system is derived from a particular organism including an endogenous CRISPR system, such as Streptococcus pyogenes.

In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence can be any polynucleotide, such as DNA or RNA polynucleotides. In some embodiments, a target sequence is located in the nucleus or cytoplasm of a cell.

In the target nucleic acid, each protospacer is associated with a protospacer adjacent motif (PAM) whose recognition is specific to individual CRISPR systems. In the Streptococcus pyogenes CRISPR/Cas system, the PAM is the nucleotide sequence NGG. For example, in the Streptococcus thermophiles CRISPR/Cas system, the PAM is the nucleotide sequence is NNAGAAW. The tracrRNA duplex directs Cas to the DNA target containing the protospacer and the requisite PAM via heteroduplex formation between the spacer region of the crRNA and the protospacer DNA.

Typically, in the context of an endogenous CRISPR system, formation of a CRISPR complex (including a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. All or a portion of the tracr sequence may also form part of a CRISPR complex, such as by hybridization to all or a portion of a tracr mate sequence that is operably linked to the guide sequence.

There are many resources available for helping practitioners determine suitable target sites once a desired DNA target sequence is identified. For example, numerous public resources, including a bioinformatically generated lists potential sgRNAs to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequence.

b. Cas Proteins

Non-limiting examples of Cas proteins include Cas1, Cas1B, Cas2, Cas3, Cas4, Cas5, Cas6, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, homologues thereof, or modified versions thereof. In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, the Cas enzyme is a Cpf1 protein. In contrast to Cas9, which naturally requires two separate short RNAs, Cpf1 proteins, e.g., from Acidaminococcus sp (AsCpf1) and Lachnospiraceae bacterium (LbCpf1), Cpf1 naturally requires one sgRNA. Furthermore, it cuts DNA at target sites 3′ downstream of the PAM sequence in a staggering fashion, generating a 5′ overhang rather than producing blunt ends like Cas9.

In particular embodiments, the Cas enzyme is a naturally occurring Cas enzyme (with PAM sequence, sgRNA guide, cut site), is spCas9 (NGG, 20 bp, cuts 3 bp 5′ of PAM), FnCas9 (NGG, 20 bp, cuts 3 bp 5′ of PAM), SaCas9 (NNGRRT, 21 bp, cuts 3 bp 5′ of PAM), NmCas9 (NNNNGATT, 24 bp, cuts 3 bp 5′ of PAM), St1Cas9 (NNAGAAW, 20 bp, cuts 3 bp 5′ of PAM), St3Cas9 (NNNNACAC, 20 bp, cuts 3 bp 5′ of PAM), AsCPf1 (TTTV, 24 bp, cuts 19/24 bp 3′ of PAM), LbCpf1 (TTTV, 24 bp, cuts 19/24 bp 3′ of PAM), Cas13 (RNA targeting, 28 bp).

In some embodiments, the Cas enzyme is an engineered enzyme. Several groups have altered the Cas9 PAM requirements and targeting specificity. In one such study, researchers used an unbiased selection strategy to identify variants of SpCas9 and SaCas9 with more relaxed PAM sequence requirements. In line with these findings, a different study utilized a structure-guided design strategy to re-engineer FnCas9 to recognize YG PAM sequences instead of NGG (Adli, supra)

In some embodiments, the CRISPR enzyme is one that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III) can be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. Also referred to as dCas9. In some embodiments, a CRISPR enzyme is considered to substantially lack all DNA cleavage activity when the DNA cleavage activity of the mutated enzyme is less than about 25%, 10%, 5%>, 1%>, 0.1%>, 0.01%, or lower with respect to its non-mutated form.

In some embodiments, an enzyme coding sequence encoding a CRISPR enzyme is codon optimized for expression in particular cells, such as eukaryotic cells. The eukaryotic cells can be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules.

The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al., Nucl. Acids Res., 28:292 (2000). Computer algorithms for codon optimizing a particular sequence for expression in a particular host cell, for example Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a CRISPR enzyme correspond to the most frequently used codon for a particular amino acid.

In some embodiments, a vector encodes a CRISPR enzyme including one or more nuclear localization sequences (NLSs). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus.

In general, the one or more NLSs are of sufficient strength to drive accumulation of the CRISPR enzyme in a detectable amount in the nucleus of a eukaryotic cell. In general, strength of nuclear localization activity may derive from the number of NLSs in the CRISPR enzyme, the particular NLS(s) used, or a combination of these factors.

c. Exemplary Vector Design Strategies

In some embodiments, one or more vectors driving expression of one or more elements of a CRISPR system are introduced into a target cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element can be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector includes one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector includes an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector includes two or more insertion sites, each insertion site being located between two tracr mate sequences so as to allow insertion of a guide sequence at each site. In such an arrangement, the two or more guide sequences can include two or more copies of a single guide sequence, two or more different guide sequences, or combinations of these. When multiple different guide sequences are used, a single expression construct may be used to target CRISPR activity to multiple different, corresponding target sequences within a cell. For example, a single vector can include about or more than about 1, 2, 3, 4, 5, 6, 7, 8, 9, 10, 15, 20 guide sequences. In some embodiments, about or more than about 1, 2, 3, 4, 5, 6, 7, 8, 9, 10, such guide-sequence-containing vectors may be provided, and optionally delivered to a cell.

In some embodiments, a vector includes a regulatory element operably linked to an enzyme-coding sequence encoding a CRISPR enzyme, such as a Cas protein.

In some embodiments, one or more of the elements of CRISPR system are under the control of an inducible promoter, which can include inducible Cas, such as Cas9.

Cong, Science, 15:339(6121):819-823 (2013) reported heterologous expression of Cas9, tracrRNA, pre-crRNA (or Cas9 and sgRNA) can achieve targeted cleavage of mammalian chromosomes. Therefore, CRISPR system utilized in the methods disclosed herein can be encoded within a vector system which can include one or more vectors which can include a first regulatory element operably linked to a CRISPR/Cas system chimeric RNA (chiRNA or gRNA or sgRNA) polynucleotide sequence, wherein the polynucleotide sequence includes (a) a guide sequence capable of hybridizing to a target sequence in a eukaryotic cell, (b) a tracr mate sequence, and (c) a tracr sequence; and a second regulatory element operably linked to an enzyme-coding sequence encoding a CRISPR enzyme which can optionally include at least one or more nuclear localization sequences. Elements (a), (b) and (c) can arranged in a 5′ to 3 orientation, wherein the components are located on the same or different vectors of the system, wherein when transcribed, the tracr mate sequence hybridizes to the tracr sequence and the guide sequence directs sequence-specific binding of a CRISPR complex to the target sequence, and wherein the CRISPR complex can include the CRISPR enzyme complexed with (1) the guide sequence that is hybridized to the target sequence, and (2) the tracr mate sequence that is hybridized to the tracr sequence, wherein the enzyme coding sequence encoding the CRISPR enzyme further encodes a heterologous functional domain. In some embodiment, one or more of the vectors encodes also encodes a suitable Cas enzyme, for example, Cas9. The different genetic elements can be under the control of the same or different promoters.

While the specifics can be varied in different engineered CRISPR systems, the overall methodology is similar. A practitioner interested in using CRISPR technology to target a DNA sequence (identified using one of the many available online tools) can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

### 2. Exemplary CRISPR/Cas Applications Subject to Modulation

a. Gene Alteration Strategies and Donor Polynucleotides

The nuclease activity of Cas enzymes can cleave target DNA to produce single or double strand breaks in the target DNA. Double strand breaks can be repaired by the cell in one of two ways: non-homologous end joining, and homology-directed repair. In non-homologous end joining (NHEJ), the double-strand breaks are repaired by direct ligation of the break ends to one another. As such, non-specific insertions and/or deletions may occur. In homology-directed repair, a donor polynucleotide with homology to the cleaved target DNA sequence is used as a template for repair of the cleaved target DNA sequence, resulting in the transfer of genetic information from a donor polynucleotide to the target DNA. As such, new nucleic acid material can be inserted/copied into the site.

Thus, site-specific transgene integration is typically achieved by the homology-directed repair (HDR) pathway including short-fragment homologous recombination (SFHR), which is inefficient and not readily accessible to non-dividing cells. By contrast, non-homologous end joining (NHEJ), the other major double strand break (DSB) repair pathway, is active in both proliferating and post-mitotic cells, and is generally more efficient than HDR in mammalian species. Although mostly recognized as error-prone and used for generating targeted gene knockouts, studies have also demonstrated the intrinsic precision of NHEJ repair, which was successfully harnessed for gene knock-ins. For example, homology-independent targeted integration (HITI) is a NHEJ-based homology-independent strategy for targeted integration of transgenes in both dividing and non-dividing cells, that shows more robust knock-in compared with HDR- and micro-homology-mediated end-joining (MMEJ)-based methods. See, e.g., Suzuki, et al., Nature. 540(7631): 144-149. doi: 10.1038/nature20565. (2016).

Therefore, in some embodiments, the genome editing composition optionally includes a donor polynucleotide. The modifications of the target DNA due to NHEJ and/or homology-directed repair can be used to induce gene correction, gene replacement, gene tagging, transgene insertion, nucleotide deletion, gene disruption, gene mutation, etc.

Accordingly, cleavage of DNA by the genome editing composition can be used to delete nucleic acid material from a target DNA sequence (e.g., to disrupt a gene that makes cells susceptible to infection (e.g., the CCR5 or CXCR4 gene, which makes T cells susceptible to HIV infection), to remove disease-causing trinucleotide repeat sequences in neurons, to create gene knockouts and mutations as disease models in research, etc.) by cleaving the target DNA sequence and allowing the cell to repair the sequence in the absence of an exogenously provided donor polynucleotide. Thus, the subject methods can be used to knock out a gene (resulting in complete lack of transcription or altered transcription) or to knock in genetic material into a locus of choice in the target DNA.

Alternatively, if the genome editing composition includes a donor polynucleotide sequence that includes at least a segment with homology to the target DNA sequence, the methods can be used to add, i.e., insert or replace, nucleic acid material to a target DNA sequence (e.g., to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, the compositions can be used to modify DNA in a site-specific, i.e., “targeted”, way, for example gene knock-out, gene knock-in, gene editing, gene tagging, etc. as used in, for example, gene therapy, e.g., to treat a disease or as an antiviral, antipathogenic, or anticancer therapeutic.

In applications in which it is desirable to insert a polynucleotide sequence into a target DNA sequence, a polynucleotide including a donor sequence to be inserted is also provided to the cell. By a “donor sequence” or “donor polynucleotide” or “donor oligonucleotide” it is meant a nucleic acid sequence to be inserted or recombined into the cleavage site. The donor polynucleotide is designed according to the insertion or recombination strategy being used and the desired genomic alteration, and typically contains sufficient homology to a genomic sequence at the cleavage site, e.g., 70%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g., 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence includes a non-homologous sequence flanked by two regions of homology, such that homology-directed repair between the target DNA region and the two flanking sequences results in insertion of the non-homologous sequence at the target region.

Donor sequences can also include a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence can include certain sequence differences as compared to the genomic sequence, e.g., restriction sites, nucleotide polymorphisms, selectable markers (e.g., drug resistance genes, fluorescent proteins, enzymes etc.), etc., which can be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence can be a single-stranded DNA, single-stranded RNA, double-stranded DNA, or double-stranded RNA. It can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. Proc. Natd. Acad. Sci. USA 84:4959-4963 (1987); Nehls et al. Science 272:886-889 (1996). Additional methods for protecting exogenous polynucleotides from degradation include, but are not limited to, addition of terminal amino group(s) and the use of modified internucleotide linkages such as, for example, phosphorothioates, phosphor amidates, and O-methyl ribose or deoxyribose residues. Any of the donor oligonucleotides can include one or more of these optional linkages, particular between the two, three or four terminal 5′ and two, three or four terminal 3′ nucleotides. In some embodiments, the e.g., phosphorothioate internucleotide linkages need not be sequential and can be dispersed within the donor oligonucleotide. Nevertheless, in some embodiments, the modified internucleotide linkages can be oriented primarily near each termini of the donor oligonucleotide.

As an alternative to protecting the termini of a linear donor sequence, additional lengths of sequence can be included outside of the regions of homology that can be degraded without impacting recombination. A donor sequence can be introduced into a cell as part of a vector molecule having additional sequences such as, for example, replication origins, promoters and genes encoding antibiotic resistance.

b. Other Applications

In addition to cutting one or both strands of the DNA duplex and introduce mutations therein by DNA repair and/or recombination, CRISPR/Cas systems can be used to directly modify bases, regulate gene expression, edit the epigenome, monitor and manipulate chromatin, and facilitate large scale screens. See, e.g., Adli, supra.

For example, the nickase Cas9 is the foundational platform for the base editor tools that enables direct C to T or A to G conversion at the target site without DSBs. A fusion complex composed of nickase Cas9 fused to an APOBEC1 deaminase enzyme and Uracyl Glycosylase inhibitor (UGI) protein effectively converts Cytosine (C) into Thymine (T) at the target site without causing double strand DNA breaks, and a transfer RNA adenosine deaminase has also been evolved and fused to nickase Cas9 to develop another base editor that achieves direct A-G conversion at the target sites.

dCas9 strongly binds to the DNA target sequence and this tight binding interferes with the activity of other DNA binding proteins such as endogenous transcription factors and RNA Polymerase II. This has been exploited to develop the CRISPR interference (CRISPRi) approach in which dCas9 binding activity blocks the transcriptional process and thus knocks down (KD) gene expression. Fusing a strong repressor complex such as Kruppel-associated Box (KRAB) to dCas9 results in a stronger and more specific gene repressor than dCas9 alone. The repression module of the KRAB protein, which is present in a large fraction of human zinc finger transcription factors, is ˜45 amino acid (a.a.). The KRAB-containing zinc finger proteins constitute the largest family of transcriptional repressors in mammals. These transcriptional regulators further recruit additional co-repressor proteins such as KRAB-box-associated protein-1 (KAP-1) and epigenetic readers such as heterochromatin protein 1 (HP1) proteins to repress genes.

In contrast to dCas9-KRAB-mediated gene repression, using the dCas9-targeting platform to recruit strong transcriptional activators results in robust induction of gene expression. The initial studies fused dCas9 to VP64, which is composed of four tandem copies of a 16-amino-acid-long transactivation domain (VP16) of the Herpes simplex virus. The dCas9-VP64-mediated gene activation strategy was further improved by a number of second-generation CRISPR-based gene activation platforms. In addition to fusing dCas9 with various copies of the VP16 protein, dCas9 was also fused to a tripartite transactivation complex, which is composed of VP64, P65, and Rta (VPR) proteins, to achieve robust gene induction. P65 is a transcription activation domain of the mammalian NF-κB transcription factor, whereas Rta is an R transactivator (Rta) from the Epstein-Bar virus.

In addition to fusing to dCas9, the effector domains can also be recruited directly to the sgRNA scaffold. For these approaches, the sgRNA scaffold is engineered to contain RNA modules such as MS2 hairpin aptamers that can bind to specific RNA binding proteins such as bacteriophage MS2-coat protein (MCP). For example, an engineered sgRNA-MS2 scaffold was used to recruit MCP-fused VP64 or the P65-HSF1 transactivation complex (HSF1: heat shock transcription activator) to activate expression from an endogenous locus. In another approach called a synergistic activation mediator (SAM) complex, in addition to dCas9-VP64 fusion complex, MCP-fused P65-HSF1 transactivation domains were recruited to the target site through the engineered sgRNA scaffold. Additionally, in a strategy termed SunTag, dCas9 fused protein scaffold that contains repeating peptide array was used to recruit multiple copies of an antibody fused effector protein.

CRISPR/Cas has also been used to both deposit DNA methylation marks as well as remove the endogenous DNA methylation from the target site. To deposit DNA methylation at a specifically targeted locus, dCas9 can be fused to the catalytic domain of eukaryotic DNA methyl transferase (DNMT3A) or prokaryotic DNA methyltransferase (MQ3). Results show, that in both strategies, substantial deposition of DNA methylation and altered gene expression were observed at the target site. Importantly, targeted recruitment of additional components of repressive epigenetic machinery such as KRAB-ZNF, DNMT3L and polycomb complexes further enhanced the robustness of DNA methylation and long-term sustained gene repression. Alternatively, locus-specific DNA demethylation can be achieved by using guidable dCas9 as a platform to recruit the catalytic domains of TET proteins. In one example, a dCas9-TET1 fusion complex resulted in DNA demethylation in up to 90% of local CpG dinucleotides and a substantial increase in mRNA expression at the target sites.

Chromatin modifications are dynamically regulated by various epigenetic writers, readers, and the dCas9 platform has been used to recruit various histone modifiers, e.g., LSD1, PRDM9 methyltransferase, histone deacetylases (HDAC), Fkbp/Frb-based inducible recruitment for epigenome editing by Cas9 (FIRE-Cas9), etc., to deposit histone methylation or acetylation, remove such marks, and otherwise modulate chromatin modifications at specific loci.

CRISPR/Cas can also be used for imaging by, for example, drawing fluorophores to repetitive or non-repetitive genetic elements using one or more sgRNA to target the fluorophore to the desired location(s). This approach has been used to track native chromatin loci throughout the cell cycle and determine differential positioning of transcriptionally active and inactive regions in the 3D nuclear space.

CRISPR/Cas can also be used for directed engineering of chromatin loop structures. Targeted engineering of artificial chromatin loops between regulatory genomic regions provides a means to manipulate endogenous chromatin structures leading to, for example, formation of new enhancer-promoter connections to overcome certain genetic deficiencies or inhibit an aberrantly active enhancer-promoter interaction.

In addition to targeted genetic and epigenetic manipulations, the gene-targeting capacity of CRISPR can be utilized in large-scale functional screenings. In such applications, instead of using a single sgRNA, WT Cas9 or dCas9-effector fusion proteins are guided with hundreds or thousands of individual sgRNAs in a population of cells. The ultimate aim for such studies is to identify genes that influence a specific phenotype in an unbiased fashion. Once established, such an approach becomes a powerful high-throughput assay to functionally screen a large number of genes at the same time.

c. CRISPR RNA/ssPNA Duplexes

CRISPR RNA/PNA duplexes are provided. Typically, the CRISPR RNA/PNA duplexes include a spacer sequence-containing CRISPR RNA having a spacer sequence that is complimentary to a protospacer sequence of a target genomic DNA, where the spacer sequence includes from 15 to 25 contiguous nucleobases, inclusive, or any subrange of specific integers there between; and (b) an anti-spacer ssPNA oligomer, where the ssPNA includes 5-25 contiguous nucleobases, inclusive, or any subrange of specific integers there between, and where at least 50%, up to 100% of the PNA nucleobases are complimentary to the nucleotides in the spacer sequence, and where the at least 50% up to 100% of the ssPNA nucleobases are hybridized to the complimentary nucleotides in the spacer sequence. Typically, the PNA is hybridized to the spacer sequence by Watson-Crick binding only.

In some embodiments, the ssPNA is hybridized to 1, 2, 3, 4, 5, 6, 7, 8, 9, 10, 11, 12, 13 14, 15, 16, 17, 18, 19, 20, 21, 22, 23, 24 or 25 nucleotides of the CRISPR RNA spacer sequence. In some embodiments, the hybridization includes 0, 1, 2, 3, 4, 5 or more mismatches, gaps, and/or insertions. In some embodiments, the ssPNA is hybridized to all the nucleotides in the spacer sequence. In some embodiments, the CRISPR RNA is or includes a crRNA, a tracrRNA, a guide RNA (gRNA) or a single guide RNA (sgRNA). In some embodiments, the PNA is or includes 10 or 20 nucleic acids in length. In some embodiments, the CRISPR RNA/PNA duplex exhibits reduced binding to the protospacer sequence, and optionally cannot bind to the protospacer sequence as compared to a corresponding CRISPR RNA in the absence of the ssPNA. In some embodiments, the PNA includes at least an integer number between 1 and 25, inclusive, of bases between −20 and −1 of the target site's protospacer sequence, wherein −1 is the last nucleobase of the protospacer and +1 is the first nucleobase of the ssPAM sequence. In some embodiments, the wherein some or all of the ssPNA residues are modified at the gamma position. Exemplary modifications include diethylene glycol, or substitution with an amino acid side chain, optionally selected from alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. In other embodiments, none of the PNA residues are modified at the gamma position.

In some embodiments, the ssPNA includes one or more heterocyclic bases selected from uracil, thymine, cytosine, adenine, guanine, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-o-D-ribofuranosyl)pyridine (2-aminopyridine), and various pyrrolo- and pyrazolopyrimidine derivatives. Exemplary heterocyclic bases are selected from thymine, cytosine, adenine, and guanine.

In some embodiments, the PNA includes one or more positively charged moieties. Exemplary positively charge moieties are selected from lysine and arginine. In some embodiments, one or more positively charged moieties are present at the N-terminus, C-terminus, or both. In some embodiments, the ssPNA includes at least 20 PNA residues and one, two or three lysines at each of the N-terminus, or the and C-terminus, or both the N-terminus and C-terminus. In exemplary embodiments, the PNA includes a nucleic acid sequence of any one of the sequences set forth in Table 3 or Table 3A.

### III. Methods of Use

It has been established that synthetic high-affinity gRNA-binding ssPNA oligomers can be implemented into CRISPR/Cas editing systems to rationally manipulate Cas9 interactions with dsDNA targets. Therefore, methods of using the disclosed compositions of ssPNA oligomers to modulate the gene editing and non-gene editing applications of CRISPR/Cas in vivo, ex vivo, or in vivo are provided. The methods typically include contacting a cell with an effective amount of CRISPR/Cas elements in combination with the paired PNAs.

The PNAs can be contacted with the cells before, at the same time as, or after the CRISPR/Cas elements. In some embodiments, the cells are contacted with a pre-annealed CRISPR RNA-ssPNA complex, optionally further including a Cas protein. Example 4 shows that PNAs designed to bind to gRNAs can inhibit Cas9 binding to and cutting at its target locus across timepoints. Each condition fails to accumulate additional edits after ssPNA introduction, indicating that ssPNAs elicit their effects and persist on timescales longer than purified Cas9 RNP.

The PNAs and CRISPR/Cas elements can be deployed in the same or separate compositions.

In some embodiments, the compositions are deployed to modulate on-target activity of CRISPR/Cas technology. Modifying Cas enzyme affinity for its cognate DNA target allows for precise control over activity at that target. This application applies not only to Cas nucleases used for gene editing purposes, but also for the diverse uses of recombinant forms of nuclease-deficient Cas enzymes as a tool to localize heterologous proteins to specific sequences. A suite of recombinant Cas enzymes (e.g., dCas9 proteins) have been engineered to suppress or activate gene expression at promotors, target deaminase proteins for individual C>T nucleobase edits (Base Editor) and is used as a tool to generate histone modification at promoters, enhancers, and throughout the genome, etc. as introduced above Adli, et al., Nature Communications 9, 1911, doi:10.1038/s41467-018-04252-2 (2018). ssPNAs offer an additional layer of control by allowing a user to manipulate the timing and location at which these tools bind the genome and elicit an effect. See, e.g., Examples 1-8. In some embodiments, the compositions are deployed to modulate off-target activity of CRISPR/Cas technology. Tools for preventing the binding of Cas enzymes are also desirable for their ability to prevent deleterious binding and cutting events at “off-target” sequences in the genome, usually regions with significant homology to the on-target sequence. Generalized inhibitors of the Cas9 enzyme, as well as nuclease deficient versions of Cas9 targeted to off-target regions have been shown to reduce the occurrence of these events. This approach has the potential to be coupled to CRISPR-Cas9 based therapies as a safety measure to reduce undesired and/or deleterious genetic events.

Beyond use as a nuclease for gene knockout and editing, nuclease-deficient variants of Cas9 (dCas9) are widely used to create fusion systems for sequence-specific localization of effectors. Precise control over these systems via sequence-specific dCas9 binding modulation further expands the versatility of applications and provide improved control for biological investigations and applications.

Collectively, the data set forth in the Examples demonstrate that antispacer PNAs can temporally manipulate stable dCas9 fusion systems and influence downstream biology. The data indicate that rapid and sequence-specific PNA blockade is achieved following a single PNA dose, with full effects noted by 24-48 hours and persistence for at least an additional 2-3 days after that. Therefore, methods of administering antispacer ssPNAs to enhance control over Cas9-derived experimental systems and improve the safety of human therapeutic applications are provided. The antispacer ssPNAs target gRNA sequences and are theoretically applicable to all current and future Cas9 and dCas9-based systems as well as other RNA-guided RNP systems.

Methods for manipulating and/or modulating the gene editing and non-gene editing applications of CRISPR/Cas in vivo, ex vivo, or in vivo are typically provide a CRISPR/Cas gene editing composition modified by annealing with an anti-spacer ssPNA oligomer (PNA/CRISPR/Cas gene editing composition). The methods employ the modified PNA/CRISPR/Cas gene editing compositions for the sequence-specific inhibition of Cas gene editing, and/or to enhance the sequence-specificity of Cas gene editing, as desired. The methods can be applied to modulate gene editing of cells ex vivo, or integrated with in vivo gene therapy applications.

### A. Providing a PNA/CRISPR RNA/Cas Gene Editing Composition

In some embodiments, the methods include formation of a PNA/CRISPR/Cas gene editing composition. Forming a PNA/CRISPR RNA/Cas gene editing composition typically includes one or more steps of designing the ssPNA, forming a ssPNA/CRISPR RNA complex and forming a ssPNA/CRISPR RNA/Cas complex.

1. Designing ssPNAs

In some embodiments, the methods include one or more steps of designing ssPNAs. Typically, ssPNAs are designed to bind to a specific CRISPR RNA that includes a spacer sequence (e.g., crRNA, a tracrRNA, a guide RNA (gRNA) or single guide RNA (sgRNA)), at a specific location within the spacer sequence. Since the spacer sequence of a CRISPR RNA is typically complimentary to a protospacer within a target DNA, the ssPNAs are preferably designed to include all or part of the nucleic acid sequence of an on-target protospacer.

The ssPNAs are preferably designed to include 1, 2, 3, 4, 5, 6, 7, 8, 9, 10, or more nucleotides of an on-target protospacer. The PNAs can be designed to include part or all of the on-target protospacer. In this way, ssPNAs may bind a CRISPR RNA including a spacer sequence, such as a gRNA spacer, to modulate the relative affinity of Cas9 for a given on-target versus off-target by limiting the spacer region of the CRISPR RNA available for binding to the genome in such as way that it favorably alters the balance of on-target/off-target editing events. In some embodiments, the PNAs show 10% to 100% percent sequence complementarity or any subrange range or specific integer there between to the on-target gRNA spacer.

In some embodiments, the PNAs are designed to target the distal end of the on-target gRNA. For example, in some embodiments, the target sequence (i.e, crRNA) of the gRNA is 15-25 nucleotides, or any subrange or specific integer there between, and the ssPNA is 5-20 PNA units or any subrange of specific integer there between, wherein the PNA binds the gRNA at least 1, 2, 3, 4, 5, 6, 7, 8, 9, 10, 11, 12, 13, 14, 15, 16, 17, 18, 19, or 20 nucleotides distal to the PAM site. In an exemplary embodiment, the target sequence (i.e, crRNA) of the gRNA is 20 nucleotides, the ssPNA is 10 PNA units (i.e., 10mer) and the PNA binds the gRNA 10 nucleotides distal to the PAM site.

The contacting can occur in vitro, ex vivo, or in vivo. In some embodiments, the method includes contacting a population of target cells with an effective amount of gene editing CRISPR/Cas composition to accomplish the desired result, in combination with an effective amount of PNAs to alter the spatial and/or temporal activity of the CRISPR/Cas at on-target and/or off-target site(s), preferably without eliminating the desired result. For example, in some embodiments, the spatial and/or temporal activity (e.g., editing) of the CRISPR/Cas is reduced by 5, 10, 15, 20, 25, 30, 35, 40, 45, 50, 55, 60, 65, 70, 75, 80% or more in the presence of the PNA(s) relative to the absence of the PNA(s). Desired results depend on the CRISPR/Cas system being used, as discussed in more detail elsewhere herein and/or are otherwise known to the user. Desired results include, but are not limited to, modifying the genomes of a sufficient number of cells to e.g., achieve a therapeutic result, regulation of gene expression, epigenetic editing, chromatin engineering, and imaging etc.

As demonstrated by the Examples, the antispacer PNAs can be rationally designed to impart two distinct modulation effects on Cas9 activity: superior sequence-specific inhibition via PAM-proximal spacer targeting, and affinity manipulation and specificity enhancement via PAM-distal binding. Therefore, in some embodiments, the methods modulate spatiotemporal activity of dCas9 fusion systems in a sequence-specific manner.

i. Sequence-Specific Inhibition of Cas

In some embodiments, the PNAs are designed to induce sequence-specific inhibition of CRSIPR/Cas editing applications. Typically, when sequence-specific inhibition of CRSIPR/Cas editing is desired, ssPNAs are designed to bind to the PAM-proximal region of the spacer sequence of the CRISPR RNA. As set forth in the Examples, PNAs with PAM-proximal homology were shown to induce potent Cas9 inhibition in human cells, by targeting the most consequential interactions driving R-loop formation. This approach results in superior inhibitory potency at low stoichiometric ratios with the important added benefit of sequence specificity.

In some embodiments, ssPNAs designed to bind to the PAM-proximal region of the spacer sequence of the CRISPR RNA impart sequence-specific inhibition of CRISPR/Cas editing. Therefore, in some embodiments the methods reduce or inhibit CRISPR/Cas editing of a specific sequence relative to a control. In some embodiments the methods reduce or inhibit CRISPR/Cas editing of a genomic DNA sequence by providing a ssPNA that includes a nucleic acid sequence of the PAM-proximal region of the protospacer sequence of the genomic DNA sequence. For example, in some forms, the methods reduce or inhibit CRISPR/Cas editing of a genomic DNA by providing a ssPNA that binds to the spacer sequence of the CRISP RNA that is complementary to the PAM-proximal region of the genomic DNA sequence.

In some embodiments, ssPNAs designed to bind to the PAM-proximal region of the spacer sequence of the CRISPR RNA are used as sequence-selective regulators of multiplexed Cas9 systems such as large-scale screens.

ii. Cas Sequence-Specificity Enhancement

In some embodiments, the PNAs are designed to enhance the sequence-specific activity of CRSIPR/Cas editing applications. Typically, when sequence-specific inhibition of CRSIPR/Cas editing is desired, ssPNAs are designed to bind to the PAM-distal region of the spacer sequence of the CRISPR RNA.

As set forth in the Examples, the affinity-dependent effects of PAM-distal targeted antispacer PNAs demonstrate that this approach can be leveraged to predictably improve Cas9 specificity. Because off-target sites, by definition, occur at lower affinity sites as compared to on-target loci, it is believed that specificity will increase for most Cas9 targets. However, lower affinity gRNAs were less amenable to specificity improvement using PAM-distal 10mer PNAs. Importantly, PAM-distal PNAs can be further adjusted in length and chemically modified via sidechain substitution at the gamma(γ) position in the polyamide backbone to fine-tune binding strength for improved effect.

For example, for low-affinity targets such as CCR5 and ZSCAN2, shorter 8mer or 6mer PAM-distal PNAs may result in less on-target inhibition effects and more optimally adjust specificity. Conversely, for high-affinity targets, increasing PNA affinity can further improve specificity by further reducing off-target effects and maintaining high on-target editing.

In addition to increasing PNA length beyond 10 bases, gamma(γ) position modification with chemical sidechains such as a hydroxymethly group (serine) or polyethlene glycol (mini-PEG) was shown to improve PNA helical organization and increase binding strength. Coupled with models predicting antispacer PNA influence on gRNA target affinity, provided herein, the described methods have the potential to predict optimized PNA formulations for individual gRNA targets.

In some embodiments, ssPNAs designed to bind to the PAM-distal region of the spacer sequence of the CRISPR RNA enhance the sequence-specificity CRISPR/Cas editing relative to a control. Therefore, in some embodiments the methods increase or enhance the specificity of “on-target” CRISPR/Cas editing of a specific sequence and reduce or abrogate “off-target” editing. In some embodiments the methods increase the “on-target” CRISPR/Cas editing of a genomic DNA sequence by providing a ssPNA that includes a nucleic acid sequence of the PAM-distal region of the protospacer sequence of the genomic DNA sequence. For example, in some forms, the methods reduce or inhibit off-target CRISPR/Cas editing of a genomic DNA by providing a ssPNA that binds to the spacer sequence of the CRISP RNA that is complementary to the PAM-distal region of the genomic DNA sequence.

iii. In Silico Predictive Tools

Methods of designing anti-spacer ssPNA oligomers based on in silico predictive modeling are provided.

It has been established that guide RNA affinity and character can be used to reproducibly predict how much binding of an anti-spacer ssPNA oligomer will reduce on or off-target editing. For example, it has been shown that a linear equation can be identified, in which baseline affinity or Doench affinity score can be input as value X. This finding enables computational tools to predict and identify ideal targets for the specificity improvement of ssPNA anti-spacer oligomers.

Therefore, in an exemplary embodiment, anti-spacer ssPNA oligomers are designed for preferential binding to gRNAs based on in silico modeling according to the needs of a specific CRISPR/Cas-based application. In some embodiments, anti-spacer ssPNA oligomers are designed to provide maximal inhibition of on-target binding. In other embodiments, anti-spacer ssPNA oligomers are designed to provide maximal inhibition of off-target binding. In some embodiments, anti-spacer ssPNA are designed to balance minimal on-target inhibition and maximal off-target inhibition. In some embodiments, the design of anti-spacer ssPNA oligomers includes one or more steps of accessing a database, for example, a database including information relating to one or more ssPNA oligomers, or one or more CRISPR RNAs, or both. Exemplary information relating to ssPNA oligomers and/or one or more CRISPR RNAs includes binding affinity, annealing/hybridization conditions (such as temperature), ssPNA size, charge, nucleic acid sequence, PNA unit size, quantitation of ssPNA/CRISPR RNA interactions, and information relating to the efficacy of CRISPR/Cas based editing, including maximal inhibition of on-target binding, and/or off-target binding associated with an ssPNA that binds to the CRISPR RNA. In some embodiments, the design of anti-spacer ssPNA oligomers includes one or more steps of compiling the one or more pieces of information and implementing the information to provide a score, such as a baseline affinity or Doench affinity score. Therefore, in some embodiments, anti-spacer ssPNA oligomers are designed according to a baseline affinity or Doench affinity score. In some embodiments, the methods are implemented via a computer.

2. Annealing ssPNAs with CRISPR RNAs

ssPNAs and CRISPR RNAs and other components of the CRISPR/Cas system such as Cas enzyme(s) can be contacted with cells and/or administered to a subject separately or together in any combination. In some embodiments, the methods apply or administer a gene editing CRISPR/Cas composition that has been annealed to a single stranded peptide nucleic acid (ssPNA) oligomer prior to the application or administration.

Therefore, in some embodiments, the methods include administering a composition including a spacer-sequence containing CRISPR RNA bound to an ssPNA. For example, in some embodiments, the methods apply or administer a pre-annealed complex including ssPNA hybridized to a gRNA; in other embodiments, the methods apply or administer a pre-annealed complex including ssPNA hybridized to a CRISPR/Cas composition, such as a gRNA in complex with a Cas enzyme.

Therefore, in some embodiments, any of the methods include one or more steps of annealing ssPNA with a spacer-sequence containing CRISPR RNA, such as a gRNA. Annealing can include contacting one or more ssPNAs with one or more spacer-sequence containing CRISPR RNAs under conditions that allow the ssPNA to specifically and selectively hybridize to the spacer-sequence containing CRISPR RNA by Watson-Crick binding. For example, in some embodiments, the methods include one or more steps of contacting ssPNA with a CRISPR RNA, such as an sgRNA under conditions that allow the one or more ssPNAs to anneal to the sgRNA prior to formation of the ribonucleoprotein (RNP).

The methods optionally include one or more steps to purify or isolate a hybridized ssPNA/CRISPR RNA composition from the mixture prior to application/administration. In an exemplary method, 50 pmol of sgRNA and 50 pmol of PAM-distal 10mer ssPNAs are incubated to allow annealing, e.g., in a thermocycler at 37° C. for 30 minutes in a 5 μL reaction in 1×3.1 Buffer (B7203S, NEB). In some embodiments, the methods include one or more steps to confirm that the ssPNA has annealed to sgRNAs. An exemplary method is by gel shift, e.g., on 5% polyacrylamide gel.

### 3. Formation of PNA-Ribonucleoprotein (RNP) Complex

The methods include one or more steps for forming a three-component ribonucleoprotein (RNP) complex, including a PNA/CRISPR/Cas gene editing composition. The formation of the three-component complex can be carried out in vitro or in vivo.

Each of the components of the RNP complex can be provided as a separate component, or as a mixture of two components, which are then contacted with the third component. For example, as described, in some embodiments, the ssPNA is annealed with the CRISPR RNA as a PNA/CRISPR RNA complex prior to complexing with a Cas enzyme. For example, in preferred embodiments, the methods include one or more steps of contacting a PNA/CRISPR RNA complex with a Cas enzyme. In other embodiments, the methods include contacting an ssPNA with a CRISPR RNA in complex with a Cas enzyme. In other embodiments, the methods include contacting a ssPNA with a CRISPR RNA and a Cas enzyme. Therefore, the components can be contacted simultaneously, or separated in time by a period of time, such as 1, 5, 10, 20, 24, 30, 40, 50 or 60 minutes or hours. Typically, complex formation with the Cas enzyme is carried out under conditions favorable for complex formation.

In an exemplary embodiment, the methods add 45 pmol of SpCas9 (CP02, PNA BIO) at room temperature and incubate the reaction for 10 minutes to allow RNP formation.

### 4. Contacting Target Cells

In some embodiments, the methods include one or more steps of contacting one or more cells with each of the components of the RNP complex. In some embodiments, the PNA/CRISPR RNA/Cas gene editing composition is formed prior to contacting the target cell with the intact composition. In other embodiments, one or more of the ssPNA, CRISPR RNA and Cas enzyme components are contacted with the cell as single components, under conditions that facilitate formation of the PNA/CRISPR RNA/Cas gene editing composition within the cell. In preferred embodiments, the ssPNA and CRISPR RNA are provided as a pre-annealed complex. Therefore, in some embodiments, the PNA/CRISPR RNA and Cas enzyme are contacted with the cell under conditions that facilitate formation of the PNA/CRISPR RNA/Cas gene editing composition within the cell.

The contacting can occur in vitro or in vivo.

### 5. Amounts and Dosages

In some methods, the compositions are administered in an effective amount to treat, inhibit, or alleviate one or more symptoms of a disease or disorder, or to otherwise provide a desired pharmacologic and/or physiologic effect, for example, reducing, inhibiting, or reversing one or more of the underlying pathophysiological mechanisms underlying a disease or disorder.

The formulation is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the nucleic acids. The precise dosage will vary according to a variety of factors such as subject-dependent variables (e.g., age, immune system health, clinical symptoms etc.).

The disclosed compositions can be administered or otherwise contacted with target cells once, twice, or three times daily; one, two, three, four, five, six, seven times a week, one, two, three, four, five, six, seven or eight times a month. For example, in some embodiments, the composition is administered or otherwise contacted with cells every two or three days, or on average about 2 to about 4 times about week. In some embodiments, the CRISPR/Cas compositions are administered or otherwise contacted with cells more frequently than the PNA compositions.

In some embodiments, the PNA oligomer is administered to the subject or otherwise contacted with cells prior to administration of the CRISPR/Cas technology. The PNA oligomer can be administered to the subject or otherwise contacted with cells, for example, 1, 2, 3, 4, 5, 6, 8, 10, 12, 18, or 24 hours, or 1, 2, 3, 4, 5, 6, or 7 days, or any combination thereof prior to administration of the CRISPR/Cas technology.

In some embodiments, the CRISPR/Cas composition is administered to the subject or otherwise contacted with cells prior to administration of the PNA oligomer. The CRISPR/Cas composition can be administered to the subject or otherwise contacted with cells, for example, 1, 2, 3, 4, 5, 6, 8, 10, 12, 18, or 24 hours, or 1, 2, 3, 4, 5, 6, or 7 days, or any combination thereof prior to administration of the PNA oligomer.

In general, by way of example only, dosage forms useful in the disclosed methods can include doses in the range of about 10² to about 10⁵⁰, or about 105 to about 10⁴⁰, or about 1010 to about 10³⁰, or about 10¹² to about 10²⁰ copies of the CRISPR/Cas composition and/or PNA oligomer per dose. In particular embodiments, about 10¹¹, 10¹², 10¹³, 10¹⁴, 10¹⁵, 10¹⁶, or 10¹⁷ copies are administered to a subject in need thereof or otherwise contacted with cells.

In other embodiments, dosages are expressed in moles. For example, in some embodiments, the dose of CRISPR/Cas composition and/or PNA oligomer is about 0.1 pmol to about 100 nmol, or any subrange or specific dosage there between.

In some embodiments, when a CRISPR RNA and an ssPNA are to be annealed together prior to formation of a RNP in vitro, or ex vivo, the molar ratio of each component is approximately 1:1. For example, in particular embodiments, an amount of a 50 pmol CRISPR RNA is annealed to an amount of 50 pmol ssPNA. In other particular embodiments, an amount of a 100 pmol CRISPR RNA is annealed to an amount of 100 pmol ssPNA. In other particular embodiments, an amount of a 150 pmol CRISPR RNA is annealed to an amount of 150 pmol ssPNA.

In other embodiments, dosages are expressed as a function of the level or amount of the function or activity that is desired. Typically, functions or activity are measured relative to a corresponding control, such as a subject that received the same amount of a CRISPR RNA in the absence of a ssPNA oligomer. For example, in some embodiments, dosages are expressed as an amount of inhibition of on-target and/or off-target activity that is desired. Therefore, in some embodiments, dosages are expressed as an amount effective to inhibit off-target binding of CRISPR RNA, such as gRNA, by 10-fold, 20-fold, 50-fold, 100-fold, 200-fold, 300-fold, 400-fold, 500-fold, 1000-fold, 2000-fold, 3000-fold or 5000-fold, relative to a control. In some embodiments, dosages are expressed as an amount effective to inhibit on-target binding of CRISPR RNA, such as gRNA, by 10-fold, 20-fold, 50-fold, 100-fold, 200-fold, 300-fold, 400-fold, 500-fold, 1000-fold, 2000-fold, 3000-fold or 5000-fold, relative to a control. In some embodiments, dosages are expressed as an amount effective to inhibit gene-editing by a CRISPR/Cas composition, such as RNP, by 10-fold, 20-fold, 50-fold, 100-fold, 200-fold, 300-fold, 400-fold, 500-fold, 1000-fold, 2000-fold, 3000-fold or 5000-fold, relative to a control. In other embodiments, dosages are expressed as an amount effective to achieve a percentage reduction in off-target editing, for example, as compared to a control having 100% off-target editing. Therefore, in some embodiments, dosages are expressed as an amount effective to reduce off-target editing by at least 50%, 60%, 70%, 80%, 90%, 95%, 96%, 97%, 98%, 99% or more than 99%, such as 100%, relative to a control.

In other embodiments, dosages are expressed in molecules per target cells. For example, in some embodiments, the dose of CRISPR/Cas composition and/or PNA oligomer is about 10² to about 10⁵⁰, or about 105 to about 10¹⁵, or about 10⁷ to about 10¹², or about 10⁸ to about 10¹¹ copies per target cell.

In other embodiments, dosages are expressed in mg/kg, particularly when expressed as an in vivo dosage of CRISPR/Cas composition and/or PNA oligomer and packaged in a nanoparticle. Dosages can be, for example 0.1 mg/kg to about 1,000 mg/kg, or any subrange or specific dosage there between.

In other embodiments, dosages are expressed in mg/ml, particularly when the expressed as an ex vivo dosage of CRISPR/Cas composition and/or PNA oligomer packaged in a nanoparticle. Dosages can be, for example 0.01 mg/ml to about 100 mg/ml, or about 0.5 mg/ml to about 50 mg/ml, or about 1 mg/ml to about 10 mg/ml per dose to a cell population of 10⁶ cells.

As discussed above, CRISPR/Cas and/or PNA oligomer can be administered or otherwise contacted with cells with or without at least one donor oligonucleotide. Such donors can be administered at similar dosages as provided for the CRISPR/Cas and PNA composition. Compositions should include an amount of donor fragment effective to recombine at the target site in the presence of CRISPR/Cas mediated cutting of the target DNA.

B. Ex vivo Gene Therapy

In some embodiments, ex vivo gene therapy of cells is used for the treatment of a genetic disorder in a subject or research purposes. For ex vivo gene therapy, cells are isolated from a subject and contacted ex vivo with the compositions to produce cells containing mutations in or adjacent to genes, or other CRISPR/Cas-based manipulation of the cells.

Therefore, methods of using the disclosed compositions of ssPNA oligomers to modulate the gene editing and non-gene editing applications of CRISPR/Cas in ex vivo gene therapy are provided. The methods typically include contacting a cell ex vivo with an effective amount of CRISPR/Cas elements in combination with the paired PNAs.

In a preferred embodiment, cells are isolated from the subject to be treated or from a syngenic host. Target cells are removed from a subject prior to contacting with disclosed composition. The cells can be, for example, hematopoietic progenitor or stem cells, T-cells, myeloid cells, NK cells, or macrophages. In a preferred embodiment, the target cells are CD34⁺ hematopoietic stem cells. Hematopoietic stem cells (HSCs), such as CD34⁺ cells are multipotent stem cells that give rise to all the blood cell types including erythrocytes. Therefore, CD34⁺ cells can be isolated from a patient with, for example, thalassemia, sickle cell disease, or a lysosomal storage disease, the mutant gene altered or repaired ex-vivo using the disclosed compositions and methods, and the cells reintroduced back into the patient as a treatment or a cure.

Stem cells can be isolated and enriched by one of skill in the art. Methods for such isolation and enrichment of CD34⁺ and other cells are known in the art and disclosed for example in U.S. Pat. Nos. 4,965,204; 4,714,680; 5,061,620; 5,643,741; 5,677,136; 5,716,827; 5,750,397 and 5,759,793. As used herein in the context of compositions enriched in hematopoietic progenitor and stem cells, “enriched” indicates a proportion of a desirable element (e.g. hematopoietic progenitor and stem cells) which is higher than that found in the natural source of the cells. A composition of cells may be enriched over a natural source of the cells by at least one order of magnitude, preferably two or three orders, and more preferably 10, 100, 200 or 1000 orders of magnitude.

In humans, CD34⁺ cells can be recovered from cord blood, bone marrow or from blood after cytokine mobilization effected by injecting the donor with hematopoietic growth factors such as granulocyte colony stimulating factor (G-CSF), granulocyte-monocyte colony stimulating factor (GM-CSF), stem cell factor (SCF) subcutaneously or intravenously in amounts sufficient to cause movement of hematopoietic stem cells from the bone marrow space into the peripheral circulation. Initially, bone marrow cells may be obtained from any suitable source of bone marrow, e.g. tibiae, femora, spine, and other bone cavities. For isolation of bone marrow, an appropriate solution may be used to flush the bone, which solution will be a balanced salt solution, conveniently supplemented with fetal calf serum or other naturally occurring factors, in conjunction with an acceptable buffer at low concentration, generally from about 5 to 25 mM. Convenient buffers include Hepes, phosphate buffers, lactate buffers, etc.

Cells can be selected by positive and negative selection techniques. Cells can be selected using commercially available antibodies which bind to hematopoietic progenitor or stem cell surface antigens, e.g. CD34, using methods known to those of skill in the art. For example, the antibodies may be conjugated to magnetic beads and immunogenic procedures utilized to recover the desired cell type. Other techniques involve the use of fluorescence activated cell sorting (FACS). The CD34 antigen, which is found on progenitor cells within the hematopoietic system of non-leukemic individuals, is expressed on a population of cells recognized by the monoclonal antibody My-10 (i.e., express the CD34 antigen) and can be used to isolate stem cell for bone marrow transplantation. My-10 deposited with the American Type Culture Collection (Rockville, Md.) as HB-8483 is commercially available as anti-HPCA 1. Additionally, negative selection of differentiated and “dedicated” cells from human bone marrow can be utilized, to select against substantially any desired cell marker. For example, progenitor or stem cells, most preferably CD34⁺ cells, can be characterized as being any of CD3⁻, CD7⁻, CD8⁻, CD10⁻, CD14⁻, CD15⁻, CD19⁻, CD20⁻, CD33⁻, Class II HLA⁺ and Thy-1⁺.

Once progenitor or stem cells have been isolated, they may be propagated by growing in any suitable medium. For example, progenitor or stem cells can be grown in conditioned medium from stromal cells, such as those that can be obtained from bone marrow or liver associated with the secretion of factors, or in medium including cell surface factors supporting the proliferation of stem cells. Stromal cells may be freed of hematopoietic cells employing appropriate monoclonal antibodies for removal of the undesired cells.

The isolated cells are contacted ex vivo with the disclosed composition in amounts effective to cause the desired mutations in or adjacent to genes in need of repair or alteration. These cells are referred to herein as modified cells. Methods for transfection of cells with proteins, nucleic acids, and peptide nucleic acids are well known in the art (Koppelhus, et al., Adv. Drug Deliv. Rev., 55(2): 267-280 (2003)). It may be desirable to synchronize the cells in S-phase to further increase the frequency of gene correction. Methods for synchronizing cultured cells, for example, by double thymidine block, are known in the art (Zielke, et al., Methods Cell Biol., 8:107-121 (1974)).

The modified cells can be maintained or expanded in culture prior to administration to a subject. Culture conditions are generally known in the art depending on the cell type. Conditions for the maintenance of CD34⁺ in particular have been well studied, and several suitable methods are available. A common approach to ex vivo multi-potential hematopoietic cell expansion is to culture purified progenitor or stem cells in the presence of early-acting cytokines such as interleukin-3. It has also been shown that inclusion, in a nutritive medium for maintaining hematopoietic progenitor cells ex vivo, of a combination of thrombopoietin (TPO), stem cell factor (SCF), and flt3 ligand (Flt-3L; i.e., the ligand of the flt3 gene product) was useful for expanding primitive (i.e., relatively non-differentiated) human hematopoietic progenitor cells in vitro, and that those cells were capable of engraftment in SCID-hu mice (Luens et al., 1998, Blood 91:1206-1215). In other known methods, cells can be maintained ex vivo in a nutritive medium (e.g., for minutes, hours, or 3, 6, 9, 13, or more days) including murine prolactin-like protein E (mPLP-E) or murine prolactin-like protein F (mPIP-F; collectively mPLP-E/IF) (U.S. Pat. No. 6,261,841). It will be appreciated that other suitable cell culture and expansion method can be used in accordance with the descripion as well. Cells can also be grown in serum-free medium, as described in U.S. Pat. No. 5,945,337.

In another embodiment, the modified hematopoietic stem cells are differentiated ex vivo into CD4⁺ cells culture using specific combinations of interleukins and growth factors prior to administration to a subject using methods well known in the art. The cells may be expanded ex vivo in large numbers, preferably at least a 5-fold, more preferably at least a 10-fold and even more preferably at least a 20-fold expansion of cells compared to the original population of isolated hematopoietic stem cells.

In another embodiment cells for ex vivo gene therapy, the cells to be used can be dedifferentiated somatic cells. Somatic cells can be reprogrammed to become pluripotent stem-like cells that can be induced to become hematopoietic progenitor cells. The hematopoietic progenitor cells can then be treated with the disclosed compositions as described above with respect to CD34⁺ cells to produce recombinant cells having one or more modified genes. Representative somatic cells that can be reprogrammed include, but are not limited to fibroblasts, adipocytes, and muscles cells. Hematopoietic progenitor cells from induced stem-like cells have been successfully developed in the mouse (Hanna, J. et al. Science, 318:1920-1923 (2007)).

To produce hematopoietic progenitor cells from induced stem-like cells, somatic cells are harvested from a host. In a preferred embodiment, the somatic cells are autologous fibroblasts. The cells are cultured and transduced with vectors encoding Oct4, Sox2, Klf4, and c-Myc transcription factors. The transduced cells are cultured and screened for embryonic stem cell (ES) morphology and ES cell markers including, but not limited to AP, SSEA1, and Nanog. The transduced ES cells are cultured and induced to produce induced stem-like cells. Cells are then screened for CD41 and c-kit markers (early hematopoietic progenitor markers) as well as markers for myeloid and erythroid differentiation.

The modified hematopoietic stem cells or modified induced hematopoietic progenitor cells are then introduced into a subject. Delivery of the cells may be effected using various methods and includes most preferably intravenous administration by infusion as well as direct depot injection into periosteal, bone marrow and/or subcutaneous sites.

The subject receiving the modified cells may be treated for bone marrow conditioning to enhance engraftment of the cells. The recipient may be treated to enhance engraftment, using a radiation or chemotherapeutic treatment prior to the administration of the cells. Upon administration, the cells will generally require a period of time to engraft. Achieving significant engraftment of hematopoietic stem or progenitor cells typically takes weeks to months.

A high percentage of engraftment of modified hematopoietic stem cells is not envisioned to be necessary to achieve significant prophylactic or therapeutic effect. It is expected that the engrafted cells will expand over time following engraftment to increase the percentage of modified cells. In some embodiments, the modified cells have a corrected α-L-iduronidase gene. Therefore, in a subject with Hurler syndrome, the modified cells are expected to improve or cure the condition. It is believed that engraftment of only a small number or small percentage of modified hematopoietic stem cells will be required to provide a prophylactic or therapeutic effect.

In preferred embodiments, the cells to be administered to a subject will be autologous, e.g. derived from the subject, or syngenic.

C. In vivo Gene Therapy

The disclosed compositions can be administered directly to a subject for in vivo gene therapy, or other CRISPR/Cas-based manipulation.

Methods of using the disclosed compositions of ssPNA oligomers to modulate the gene editing and non-gene editing applications of CRISPR/Cas for in vivo gene therapy are provided. The methods typically include contacting a target cell in vivo with an effective amount of CRISPR/Cas elements in combination with the paired PNAs. Therefore, in some embodiments, the methods modulate the localization and/or activity of CRISPR/Cas systems used for gene therapy in vivo.

### 1. Adjunct Therapy

In some embodiments, the methods administer one or more compositions of CRISPR/Cas elements in combination with the paired PNAs as an addition to, or in combination with other CRISPR/Cas elements, such as other CRISPR/Cas elements in combination with the paired PNAs, or conventional CRISPR/Cas elements. Therefore, in some embodiments, the methods administer one or more compositions of CRISPR/Cas elements in combination with the paired PNAs as a secondary CRISPR/Cas based application, that is carried out in addition to, or in combination with a primary CRISPR/Cas based application.

The term “conventional” in relation to CRISPR/Cas elements and CRISPR/Cas-based applications refers to CRISPR/Cas elements in the absence of the described paired PNAs, and applications thereof.

The terms “primary” and “secondary” in relation to CRISPR/Cas-based applications refer to a first and second distinct administration of a composition that may include distinct amounts/dosing and timings of administration. The terms are independent of the time, order, amount, etc. of composition(s) that are administered. The term “adjunct” refers to a secondary therapy that is given in addition to a primary or initial therapy, for example, to create a desired physiological effect in the recipient that is related to the effect of the primary therapy. In some embodiments, the adjunct therapy increases or maximize the effectiveness of the primary therapy.

Therefore, methods of using the disclosed compositions of ssPNA oligomers as an adjunct to in vivo CRISPR/Cas gene editing applications, are also provided. In some embodiments, the methods administer compositions of ssPNA oligomers before, during or after one or more primary in vivo CRISPR/Cas gene editing applications. In particular embodiments, the methods administer one or more compositions of CRISPR/Cas elements in combination with the paired PNAs as an adjunct therapy before, during or after a primary CRISPR/Cas-based in vivo gene therapy that administers one or more compositions of CRISPR/Cas elements in combination with the paired PNAs. The adjunct therapy can enhance, reduce or otherwise modulate or augment the primary CRISPR/Cas-based in vivo gene therapy. In other embodiments, the methods administer one or more compositions of CRISPR/Cas elements in combination with the paired PNAs before, during or after a primary conventional CRISPR/Cas-based in vivo gene therapy, to enhance, reduce or otherwise modulate or augment the primary conventional CRISPR/Cas-based in vivo gene therapy.

a. Tissue-Specific Blockade

Methods of using the anti-spacer ssPNA oligomers for tissue-specific blockade of CRISPR/Cas-based in vivo gene editing applications are provided.

In some embodiments, the methods administer one or more compositions of CRISPR/Cas elements in combination with the paired PNAs to prevent off-target CRISPR/Cas-based editing within a non-target tissue.

The intravenous (i.v.) administration of conventional CRISPR/Cas elements targeting the lung can lead to potentially toxic editing of other tissues, including the liver. Therefore, in some embodiments, the methods administer one or more compositions of CRISPR/Cas elements in combination with the paired PNAs to prevent CRISPR/Cas editing activity in a specific tissue or organ to protect the tissue from toxic effects associated with non-target CRISPR/Cas editing of that tissue or organ. Typically, when the methods administer CRISPR/Cas elements in combination with the paired PNAs as an adjunct to protect a tissue non-target editing associated with a primary CRISPR/Cas application, the methods administer the CRISPR/Cas elements in combination with the paired PNAs prior to administering the primary CRISPR/Cas application. In an exemplary embodiment, the methods administer one or more compositions of CRISPR/Cas elements in combination with the paired PNAs to prevent CRISPR/Cas editing activity in the liver tissue prior to a primary in vivo CRISPR/Cas gene therapy targeting the lung. In some embodiments, the methods target CRISPR/Cas elements to a specific organ or tissue using one or more tissue-specific homing molecules.

Exemplary tissues and organs that can be targeted include capillaries, joints, nerves, skin, tendons, arteries, cerebellum, liver, spleen, tongue, Appendix, diaphragm, lungs, ovaries, thyroid, adrenal glands, larynx, esophagus, stomach, brain, eyes, ligaments, spinal cord, thymus gland, bones, lymph nodes, pancreas, small intestine, large intestine, bronchi, pharynx, bladder, placenta, skeletal muscle, uterus, bone marrow, heart, prostate, hair follicle, subcutaneous tissue, veins, colon, hypothalamus, mammary glands, pituitary gland, tonsils, kidneys and testes.

b. In Vivo Quenching Methods of using the anti-spacer ssPNA oligomers for in vivo quenching of CRISPR/Cas-based in vivo gene editing applications are provided.

In some embodiments, the methods administer one or more compositions of PNAs in vivo as at a specific time point following a primary CRISPR/Cas-based in vivo gene editing application, to inhibit further CRISPR/Cas-based activity. Therefore, in some embodiments, the methods administer one or more compositions of anti-spacer ssPNA oligomers in vivo to inhibit CRISPR/Cas-based activity at a desired time point.

Inhibitory anti-spacer ssPNA oligomers administered or released at a time point after initiation of CRISPR/Cas-based in vivo gene editing treatment can optimize the gene-editing timeframe, for example, to prevent further Cas gene editing activity after a certain window and thereby prevent the likelihood of off-target effects or toxicities related to persistent Cas9 activity. For example, in some embodiments, anti-spacer ssPNA oligomers are used to quench Cas gene editing activity to reduce toxicity and enhance the safety or reduce the risk associated with a given CRISPR/Cas-based application. Enhancing the safety profile associated with CRISPR/Cas-based applications may be especially important for applications where there exists a potential for persistent expression of Cas, such as viral delivery of CRISPR/Cas elements (e.g., AAV-based delivery systems), as opposed to systems that deliver finite amounts of the active agents (e.g., nanoparticle-based delivery systems).

c. Targeted In Vivo Delivery of PNA Oligomers

Methods of using the anti-spacer ssPNA oligomers for in vivo delivery of directly attached accessory molecules are provided.

In some embodiments the methods deliver anti-spacer ssPNA oligomers through direct conjugation to one or more accessory molecules that homes to a specific tissue or organ. For example, in some embodiments, conjugation to a tissue specific molecule enhances uptake and/or homing to a specific tissue or organ or group of tissue or organs.

It has been shown that nucleic acids conjugated to triantennary N-acetyl galactosamine (GalNAc), the ligand recognized by the asialoglycoprotein receptor (ASGPR), are preferentially taken up by cells that express this receptor at the cell surface. (Bhingardeve, et al., J. Org. Chem. 85, 14, 8812-8824 (2020)). Therefore, in an exemplary embodiment, anti-spacer ssPNA oligomers are directly conjugated to one or more carbohydrate moieties, such as a GalNAc sugar, for preferential uptake from the bloodstream into the liver, and/or cells bearing the asialoglycoprotein receptor (ASGPR).

### 2. Pharmaceutical Formulations

The disclosed compositions are preferably employed for therapeutic uses in combination with a suitable pharmaceutical carrier. Such compositions include an effective amount of the composition, and a pharmaceutically acceptable carrier or excipient.

It is understood by one of ordinary skill in the art that nucleotides administered in vivo are taken up and distributed to cells and tissues (Huang, et al., FEBS Lett., 558(1-3):69-73 (2004)). For example, Nyce, et al. have shown that antisense oligodeoxynucleotides (ODNs) when inhaled bind to endogenous surfactant (a lipid produced by lung cells) and are taken up by lung cells without a need for additional carrier lipids (Nyce, et al., Nature, 385:721-725 (1997)). Small nucleic acids are readily taken up into T24 bladder carcinoma tissue culture cells (Ma, et al., Antisense Nucleic Acid Drug Dev., 8:415-426 (1998)).

The disclosed compositions may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation.

Various methods for nucleic acid delivery are described, for example, in Sambrook et al., Molecular Cloning: A Laboratory Manual, Cold Spring Harbor Laboratory, New York (1989); and Ausubel, et al., Current Protocols in Molecular Biology, John Wiley & Sons, New York (1994). Such nucleic acid delivery systems include the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. The nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, optionally with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents and inert gases. In addition, sterile, fixed oils are conventionally employed as a solvent or suspending medium. For this purpose any bland fixed oil including synthetic mono- or di-glycerides may be employed. In addition, fatty acids such as oleic acid may be used in the preparation of injectables. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The disclosed compositions alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, the compositions include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the compositions are conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett., 14(19):4975-4977 (2004)) and in vivo (Soutschek, et al., Nature, 432(7014):173-178 (2004)). In addition, it has been shown that binding of steroid conjugated oligonucleotides to different lipoproteins in the bloodstream, such as LDL, protect integrity and facilitate biodistribution (Rump, et al., Biochem. Pharmacol., 59(11):1407-1416 (2000)). Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe(II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also describes methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

### 3. Particle Formulations

The compound may also be encapsulated in suitable biocompatible microcapsules, microparticles, nanoparticles, or microspheres formed of biodegradable or non-biodegradable polymers or proteins or liposomes for targeting to cells.

The polymer that forms the core of the particle may be any biodegradable or non-biodegradable synthetic or natural polymer. In a preferred embodiment, the polymer is a biodegradable polymer.

Particles are ideal materials for the fabrication of gene editing delivery vehicles: 1) control over the size range of fabrication, down to 100 nm or less, an important feature for passing through biological barriers; 2) reproducible biodegradability without the addition of enzymes or cofactors; 3) capability for sustained release of encapsulated, protected nucleic acids over a period in the range of days to months by varying factors such as the monomer ratios or polymer size, for example, the ratio of lactide to glycolide monomer units in poly(lactide-co-glycolide) (PLGA); 4) well-understood fabrication methodologies that offer flexibility over the range of parameters that can be used for fabrication, including choices of the polymer material, solvent, stabilizer, and scale of production; and 5) control over surface properties facilitating the introduction of modular functionalities into the surface.

Any number of biocompatible polymers can be used to prepare the particles. In one embodiment, the biocompatible polymer(s) is biodegradable. In another embodiment, the particles are non-degradable. In other embodiments, the particles are a mixture of degradable and non-degradable particles.

Examples of preferred biodegradable polymers include synthetic polymers that degrade by hydrolysis such as poly(hydroxy acids), such as polymers and copolymers of lactic acid and glycolic acid, other degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), and poly(amine-co-ester) polymers, such as those described in Zhou, et al., Nature Materials, 11(1):82-90 (2011), Tietjen, et al. Nature Communications, 8:191 (2017) doi:10.1038/s41467-017-00297-x, and WO 2013/082529, U.S. Published Application No. 2014/0342003, and PCT/US2015/061375.

Preferred natural polymers include alginate and other polysaccharides, collagen, albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Exemplary polymers include, but are not limited to, cyclodextrin-containing polymers, in particular cationic cyclodextrin-containing polymers, such as those described in U.S. Pat. No. 6,509,323,

In some embodiments, non-biodegradable polymers can be used, especially hydrophobic polymers. Examples of preferred non-biodegradable polymers include ethylene vinyl acetate, poly(meth) acrylic acid, copolymers of maleic anhydride with other unsaturated polymerizable monomers, poly(butadiene maleic anhydride), polyamides, copolymers and mixtures thereof, and dextran, cellulose and derivatives thereof.

Other suitable biodegradable and non-biodegradable polymers include, but are not limited to, polyanhydrides, polyamides, polycarbonates, polyalkylenes, polyalkylenes such as polyethylene and polypropylene, polyalkylene glycols such as poly(ethylene glycol) (PEG), polyalkylene oxides (PEO), polyalkylene terephthalates such as poly(ethylene terephthalate) and ethylene vinyl acetate polymer (EVA), polyvinyl alcohols, polyvinyl ethers, polyvinyl esters such as poly(vinyl acetate), polyethylene, polypropylene, poly(vinyl acetate), poly vinyl chloride, polystyrene, polyvinyl halides such as poly(vinyl chloride) (PVC), polyvinylpyrrolidone, polysiloxanes, polyvinylpyrrolidone, polymers of acrylic and methacrylic esters, polysiloxanes, polyurethanes and copolymers thereof, modified celluloses, alkyl cellulose, hydroxyalkyl celluloses, cellulose ethers, cellulose esters, nitro celluloses, hydroxypropylcellulose, cellulose acetate, cellulose propionate, cellulose acetate butyrate, cellulose acetate phthalate, carboxyethyl cellulose, cellulose triacetate, cellulose sulfate sodium salt, and polyacrylates such as poly(methyl methacrylate), poly(ethylmethacrylate), Poly(2-hydroxyethyl methacrylate) (pHEMA), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate). These materials may be used alone, as physical mixtures (blends), or as co-polymers.

The polymer may be a bioadhesive polymer that is hydrophilic or hydrophobic. Hydrophilic polymers include CARBOPOL™ (a high molecular weight, crosslinked, acrylic acid-based polymers such as those manufactured by NOVEON™), polycarbophil, cellulose esters, and dextran. polymers of acrylic acids, include, but are not limited to, poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate) (jointly referred to herein as “polyacrylic acids”).

Release rate controlling polymers may be included in the polymer matrix or in the coating on the formulation. Examples of rate controlling polymers that may be used are hydroxypropylmethylcellulose (HPMC) with viscosities of either 5, 50, 100 or 4000 cps or blends of the different viscosities, ethylcellulose, methylmethacrylates, such as EUDRAGIT® RS100, EUDRAGIT® RL100, EUDRAGIT® NE 30D (supplied by Rohm America). Gastrosoluble polymers, such as EUDRAGIT® E100 or enteric polymers such as EUDRAGIT® L100-55D, L100 and S100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

These polymers can be obtained from sources such as Sigma Chemical Co., St. Louis, MO; Polysciences, Warrenton, PA; Aldrich, Milwaukee, WI; Fluka, Ronkonkoma, NY; and BioRad, Richmond, CA, or can be synthesized from monomers obtained from these or other suppliers using standard techniques.

In certain embodiments, the hydrophobic polymer is an aliphatic polyester. In preferred embodiments, the hydrophobic polymer is polyhydroxyester such as poly(lactic acid), poly(glycolic acid), or poly(lactic acid-co-glycolic acid).

Other polymers include, but are not limited to, polyalkyl cyanoacralate, polyamino acids such as poly-L-lysine (PLL), poly(valeric acid), and poly-L-glutamic acid, hydroxypropyl methacrylate (HPMA), polyorthoesters, poly(ester amides), poly(ester ethers), polydioxanone and its copolymers, polyhydroxyalkanoates, polypropylene fumarate, polyoxymethylene, poly(butyric acid), trimethylene carbonate, and polyphosphazenes.

The particles can be designed to release molecules to be encapsulated or attached over a period of days to weeks. Factors that affect the duration of release include pH of the surrounding medium (higher rate of release at pH 5 and below due to acid catalyzed hydrolysis of PLGA) and polymer composition. Aliphatic polyesters differ in hydrophobicity and that in turn affects the degradation rate. The hydrophobic poly (lactic acid) (PLA), more hydrophilic poly (glycolic acid) PGA and their copolymers, poly (lactide-co-glycolide) (PLGA) have different release rates. The degradation rate of these polymers, and often the corresponding drug release rate, can vary from days (PGA) to months (PLA) and is easily manipulated by varying the ratio of PLA to PGA.

In some preferred embodiments, the particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(8-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. For example, particles can also contain one or more polymer conjugates containing end-to-end linkages between the polymer and a targeting moiety or a detectable label. For example, a modified polymer can be a PLGA-PEG-peptide block polymer.

The in vivo stability/release of the particles can be adjusted during the production by using polymers such as poly(lactide-co-glycolide) copolymerized with polyethylene glycol (PEG). If PEG is exposed on the external surface, it may increase the time these materials circulate due to the hydrophilicity of PEG.

A shell can also be formed of or contain a hyperbranched polymer (HP) with hydroxyl groups, such as a hyperbranched polyglycerol (HPG), hyperbranched peptides (HPP), hyperbranched oligonucleotides (HON), hyperbranched polysaccharides (HPS), and hyperbranched polyunsaturated or saturated fatty acids (HPF). The HP can be covalently bound to the one or more materials that form the core such that the hydrophilic HP is oriented towards the outside of the particles and the hydrophobic material oriented to form the core.

The HP coating can be modified to adjust the properties of the particles. For example, unmodified HP coatings impart stealth properties to the particles which resist non-specific protein absorption and are referred to as nonbioadhesive nanoparticles (NNPs). Alternatively, the hydroxyl groups on the HP coating can be chemically modified to form functional groups that react with functional groups on tissue or otherwise interact with tissue to adhere the particles to the tissue, cells, or extracellular materials, such as proteins. Such functional groups include, but are not limited to, aldehydes, amines, and O-substituted oximes. Particles with an HP coating chemically modified to form functional groups are referred to as bioadhesive nanoparticles (BNPs). The chemically modified HP coating of BNPs forms a bioadhesive corona of the particle surrounding the hydrophobic material forming the core. See, for example, WO 2015/172149, WO 2015/172153, WO 2016/183209, and U.S. Published Applications 2017/0000737 and 2017/0266119.

Particles can be formed of polymers fabricated from polylactides (PLA) and copolymers of lactide and glycolide (PLGA). These have established commercial use in humans and have a long safety record (Jiang, et al., Adv. Drug Deliv. Rev., 57(3):391-410); Aguado and Lambert, Immunobiology, 184(2-3):113-25 (1992); Bramwell, et al., Adv. Drug Deliv. Rev., 57(9):1247-65 (2005)). These polymers have been used to encapsulate siRNA (Yuan, et al., Jour. Nanosocience and Nanotechnology, 6:2821-8 (2006); Braden, et al., Jour. Biomed. Nanotechnology, 3:148-59 (2007); Khan, et al., Jour. Drug Target, 12:393-404 (2004); Woodrow, et al., Nature Materials, 8:526-533 (2009)). Murata, et al., J. Control. Release, 126(3):246-54 (2008) showed inhibition of tumor growth after intratumoral injection of PLGA microspheres encapsulating siRNA targeted against vascular endothelial growth factor (VEGF). However, these microspheres were too large to be endocytosed (35-45 m) (Conner and Schmid, Nature, 422(6927):37-44 (2003)) and required release of the anti-VEGF siRNA extracellularly as a polyplex with either polyarginine or PEI before they could be internalized by the cell. These microparticles may have limited applications because of the toxicity of the polycations and the size of the particles. Nanoparticles (100-300 nm) of PLGA can penetrate deep into tissue and are easily internalized by many cells (Conner and Schmid, Nature, 422(6927):37-44 (2003)).

Exemplary particles are described in U.S. Pat. Nos. 4,883,666, 5,114,719, 5,601,835, 7,534,448, 7,534,449, 7,550,154, and 8,889,117, and U.S. Published Application Nos. 2009/0269397, 2009/0239789, 2010/0151436, 2011/0008451, 2011/0268810, 2014/0342003, 2015/0118311, 2015/0125384, 2015/0073041, Hubbell, et al., Science, 337:303-305 (2012), Cheng, et al., Biomaterials, 32:6194-6203 (2011), Rodriguez, et al., Science, 339:971-975 (2013), Hrkach, et al., Sci Transl Med., 4:128ra139 (2012), McNeer, et al., Mol Ther., 19:172-180 (2011), McNeer, et al., Gene Ther., 20:658-659 (2013), Babar, et al., Proc Natl Acad Sci USA, 109:E1695-E1704 (2012), Fields, et al., J Control Release 164:41-48 (2012), and Fields, et al., Advanced Healthcare Materials, 361-366 (2015).

The core of the particles can be formed of or contain one or more poly(amine-co-ester), poly(amine-co-amide), poly(amine-co-ester-co-ortho ester) or a combination thereof. In some embodiments, the particles are polyplexes. In some embodiments, the content of a hydrophobic monomer in the polymer is increased relative the content of the same hydrophobic monomer when used to form polyplexes. Increasing the content of a hydrophobic monomer in the polymer forms a polymer that can form solid core particles in the presence of nucleic acids. Unlike polyplexes, these particles are stable for long periods of time during incubation in buffered water, or serum, or upon administration (e.g., injection) into animals. They also provide for a sustained release of nucleic acids which leads to long term activity. In some aspects, the molecular weight of the polymer is less than 5 kDa, 7.5 kDa, 10 kDa, 20 kDa, or 25 kDa. In some forms the molecular weight of the polymer is between about 1 kDa and about 25 kDa, between about 1 kDa and about 10 kDa, between about 1 kDa and about 7.5 kDa.

Suitable polymers as well as particles and polyplexes formed therefrom are disclosed in WO 2013/082529, WO 2016/183217, U.S. Published Application No. 2016/0251477, U.S. Published Application No. 2015/0073041, U.S. Published Application No. 2014/0073041, and U.S. Pat. No. 9,272,043, each of which is specifically incorporated by reference in entirety.

### 4. Methods of Administration

In general, methods of administering compounds, including oligonucleotides and related molecules, are well known in the art. In particular, the routes of administration already in use for nucleic acid therapeutics, along with formulations in current use, provide preferred routes of administration and formulation for the disclosed compositions are described above. Preferably the compositions are injected into the organism undergoing genetic manipulation, such as an animal requiring gene therapy.

The disclosed compositions can be administered by a number of routes including, but not limited to, oral, intravenous, intraperitoneal, intramuscular, transdermal, subcutaneous, topical, sublingual, rectal, intranasal, pulmonary, and other suitable means. The compositions can also be administered via liposomes. Such administration routes and appropriate formulations are generally known to those of skill in the art.

Administration of the formulations may be accomplished by any acceptable method which allows the gene editing compositions to reach their targets.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The compositions may be delivered in a manner which enables tissue-specific uptake of the agent and/or nucleotide delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the composition, over a certain time period, for example, hours, days, weeks, months or years. This may be accomplished, for example, by repeated administrations of a formulation or by a sustained or controlled release delivery system in which the compositions are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the oligonucleotides are contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation. In addition, a pump-based hardware delivery system may be used to deliver one or more embodiments.

Examples of systems in which release occurs in bursts include systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be in the form of pellets, or capsules.

Use of a long-term release implant may be particularly suitable in some embodiments. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

Active agent(s) and compositions thereof can be formulated for pulmonary or mucosal administration. The administration can include delivery of the composition to the lungs, nasal, oral (sublingual, buccal), vaginal, or rectal mucosa.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions composed of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+nebulizer (PARI Respiratory Equipment, Monterey, CA).

### D. Diseases to Be Treated

The disclosed compositions are especially useful to treat genetic deficiencies, disorders and diseases caused by mutations in single genes, for example, to correct genetic deficiencies, disorders and diseases caused by point mutations. Non-limiting examples include for example, cystic fibrosis, hemophilia, hemoglobinopathies such as sickle cell anemia and beta-thalassemia, xeroderma pigmentosum, and lysosomal storage diseases, though the strategies are also useful for treating non-genetic disease such as HIV, in the context of ex vivo-based cell modification and also for in vivo cell modification.

If the target gene contains a mutation that is the cause of a genetic disorder, then the disclosed compositions can be used for mutagenic repair that may restore the DNA sequence of the target gene to normal. The target sequence can be within the coding DNA sequence of the gene or within an intron. The target sequence can also be within DNA sequences that regulate expression of the target gene, including promoter or enhancer sequences.

If the target gene is an oncogene causing unregulated proliferation, such as in a cancer cell, then the donor oligonucleotide is useful for causing a mutation that inactivates the gene and terminates or reduces the uncontrolled proliferation of the cell. The donor oligonucleotide is also a useful anti-cancer agent for activating a repressor gene that has lost its ability to repress proliferation. The target gene can also be a gene that encodes an immune regulatory factor, such as PD-1, in order to enhance the host's immune response to a cancer.

Programmed cell death protein 1, also known as PD-1 and CD279 (cluster of differentiation 279), is a protein encoded by the PDCD1 gene. PD-1 has two ligands: PD-L1 and PD-L2. PD-1 is expressed on a subset of thymocytes and up-regulated on T, B, and myeloid cells after activation (Agata, et al., Int. Immunol., 8:765-772 (1996)). PD-1 acts to antagonize signal transduction downstream of the TCR after it binds a peptide antigen presented by the major histocompatibility complex (MHC). It can function as an immune checkpoint, by preventing the activation of T-cells, which in turn reduces autoimmunity and promotes self-tolerance, but can also reduce the body's ability to combat cancer. The inhibitory effect of PD-1 to act through twofold mechanism of promoting apoptosis (programmed cell death) in antigen specific T-cells in lymph nodes while simultaneously reducing apoptosis in regulatory T cells (suppressor T cells). Compositions that block PD-1, the PD-1 inhibitors, activate the immune system to attack tumors and are therefore used with varying success to treat some types of cancer.

Therefore, in some embodiments, compositions are used to treat cancer. The gene modification technology can be designed to reduce or prevent expression of PD-1, and administered in an effective amount to do so.

The compositions can be used as antiviral agents, for example, when designed to modify a specific a portion of a viral genome necessary for proper proliferation or function of the virus.

The disclosed compositions and methods can be further understood through the following numbered paragraphs.

1. A single stranded peptide nucleic acid (ssPNA) oligomer including a nucleobase sequence that hybridizes to part or all of the spacer sequence of a spacer-sequence containing CRISPR RNA that binds to a protospacer sequence of a CRISPR/Cas target site in a double stranded DNA.

2. The ssPNA oligomer of paragraph 1, wherein the oligomer hybridizes to the CRISPR RNA by Watson-Crick binding only.

3. The ssPNA oligomer of paragraphs 1 or 2, wherein the oligomer does not form a PNA:DNA:DNA or PNA:DNA:PNA triplex withthe CRISPR RNA or at the CRISPR/Cas target site.

4. The ssPNA oligomer of any one of paragraphs 1-3 including a nucleobase sequence that hybridizes to any integer number between 1-25, 5-25, 10-25, 15-25, 1-20, 5-20, 5-15, or 8-12 nucleotides of the CRISPR RNA spacer sequence.

5. The ssPNA oligomer of any one of paragraphs 1-4 wherein any subrange or specific integer number percentage between 25 and 100 of the PNA oligomer hybridizes to the CRISPR RNA spacer sequence.

6. The ssPNA oligomer of any one of paragraphs 1-5, wherein the hybridization includes 0, 1, 2, 3, 4, 5 or more mismatches, gaps, and/or insertions.

7. The ssPNA oligomer of any one of paragraphs 1-6, including a nucleobase sequence that includes a part or all of the protospacer sequence.

8. The ssPNA oligomer of any one of paragraphs 1-7, including a nucleobase sequence that includes all of the protospacer sequence.

9. The ssPNA oligomer of any one of paragraphs 1-6, wherein the CRISPR RNA is or includes a crRNA.

10. The ssPNA oligomer of any one of paragraphs 1-7, wherein the CRISPR RNA includes a tracrRNA.

11. The ssPNA oligomer of any one of paragraphs 1-10, wherein the CRISPR RNA is or includes a guide RNA (gRNA) or single guide RNA (sgRNA).

12. The ssPNA oligomer of any one of paragraphs 1-11, wherein the oligomer includes between about 5 and about 50 residues in length, more preferably about 8 to about 30 residues or about 10 to about 20 residues or about 8 to about 12 residues or about 10 residues in length.

13. The ssPNA oligomer of any one of paragraphs 1-12, wherein the CRISPR RNA that targets the CRISPR target site exhibits reduced binding, and optionally cannot bind to the protospacer sequence, when the ssPNA is present.

14. The ssPNA oligomer of any one of paragraphs 1-12, wherein a Cas enzyme that binds to the CRISPR RNA that targets the CRISPR target site exhibits reduced binding, and optionally cannot bind to the CRISPR/Cas target site, when the ssPNA is present.

15. The ssPNA oligomer of any one of paragraphs 1-14, wherein the oligomer includes at least an integer number between 5 and 40, inclusive, of bases between −20 and −1 of the target site's protospacer sequence, wherein −1 is the last nucleobase of the protospacer and +1 is the first nucleobase of the PAM sequence.

16. The ssPNA oligomer of any one of paragraphs 1-15, wherein the CRISPR RNA is unbound or in a riboprotein complex with a Cas protein.

17. The ssPNA oligomer of any one of paragraphs 1-16, wherein some or all of the PNA residues are modified at the gamma position.

18. The ssPNA oligomer of paragraph 17, wherein the modification is diethylene glycol, or substitution with an amino acid side chain, optionally selected from alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof.

19. The ssPNA oligomer of any one of paragraphs 1-16, wherein none of PNA residues are modified at the gamma position.

20. The ssPNA oligomer of any one of paragraphs 1-19, wherein the heterocyclic bases within the oligomer are selected from uracil, thymine, cytosine, adenine, guanine, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives.

21. The ssPNA oligomer of paragraph 20, wherein the heterocyclic bases are selected from thymine, cytosine, adenine, and guanine.

22. The ssPNA oligomer of any one of paragraphs 1-21, including one or more positively charged moieties.

23. The ssPNA oligomer of paragraph 22, wherein the positively charge moieties are selected from lysine and arginine.

24. The ssPNA oligomer of paragraphs 22 or 23, wherein one or more positively charged moieties are present at the N-terminus, C-terminus, or both.

25. The ssPNA oligomer of any one of paragraphs 1-24 including at least 20 PNA residues and one, two or three lysines at the N-terminus, or the C-terminus, or both the N-terminus and C-terminus.

26. The ssPNA oligomer of any one of paragraphs 1-25, wherein the ssPNA includes a nucleic acid sequence of any one of the sequences set forth in Table 3 or Table 3A.

27. The ssPNA oligomer of any one of paragraphs 1-26, wherein the oligomer is bound to a spacer-sequence containing CRISPR RNA.

28. The ssPNA oligomer of any one of paragraphs 1-27 packaged in a microparticle or nanoparticle, optionally wherein the particle is a liposome or polymeric particle.

29. The ssPNA oligomer of any one of paragraphs 1-28, wherein the target sequence of the spacer-sequence containing CRISPR RNA is 15-25 nucleotides, or any subrange of specific integer there between, and the ssPNA is 5-20 PNA units or any subrange of specific integer there between, wherein the ssPNA binds the spacer sequence at least 1, 2, 3, 4, 5, 6, 7, 8, 9, 10, 11, 12, 13, 14, 15, 16, 17, 18, 19, or 20 nucleotides distal to the PAM site of the CRISPR RNA.

30. The ssPNA oligomer of paragraph 29, wherein the target sequence of the spacer-sequence containing CRISPR RNA is 20 nucleotides, the ssPNA is 10 PNA units and the ssPNA binds the CRISPR RNA 0, 5, 10, 15 or 20 nucleotides distal to a nucleoside that forms the PAM site.

31. A CRISPR/Cas gene editing composition, including:
(a) the ssPNA oligomer of any one of paragraphs 1-15, or 17-30; and
(b) a spacer-sequence containing CRISPR RNA;
wherein the ssPNA is hybridized with the spacer-sequence containing CRISPR RNA; and
(c) optionally a Cas enzyme.

32. The CRISPR/Cas gene editing composition of paragraph 31, wherein the CAS enzyme is complexed with the ssPNA hybridized to the CRISPR RNA.

33. The CRISPR/Cas gene editing composition of paragraph 31 or 32, wherein the CRISPR RNA is or includes a crRNA, a tracrRNA, a guide RNA (gRNA) or single guide RNA (sgRNA).

34. The CRISPR/Cas gene editing composition of any one of paragraphs 31-33, wherein the Cas enzyme is selected from the group consisting of spCas9, FnCas9, SaCas9, NmCas9, St1Cas9, St3Cas9, AsCPf1, LbCpf1, and Cas13.

35. A pharmaceutical composition including an effective amount of the ssPNA of any one of paragraphs 1-30, or the CRISPR/Cas gene editing composition of any one of paragraphs 31 to 34, to reduce activity of the CRISPR/Cas at the target site.

36. A pharmaceutical composition including
(a) an effective amount of the ssPNA of any one of paragraphs 1-30, to reduce activity of the CRISPR/Cas at the target site; and
(b) an effective amount of the CRISPR RNA, or a nucleic acid expression construct encoding the same.

37. The pharmaceutical composition of paragraph 36, wherein the CRISPR RNA is or includes a crRNA, a tracrRNA, a guide RNA (gRNA) or single guide RNA (sgRNA).

38. A pharmaceutical composition including an effective amount of the ssPNA of any one of paragraphs 1-30, to reduce activity of the CRISPR/Cas at an off-target site of the target site.

39. The pharmaceutical composition of paragraph 38, wherein the target sequence of the CRISPR RNA is 15-25 nucleotides, or any subrange of specific integer there between, and the ssPNA is 5-20 PNA units or any subrange of specific integer there between, wherein the ssPNA binds the CRISPR RNA at least 1, 2, 3, 4, 5, 6, 7, 8, 9, 10, 11, 12, 13, 14, 15, 16, 17, 18, 19, or 20 nucleotides distal to the PAM site of the spacer-sequence containing CRISPR RNA,
optionally wherein the target sequence of the gRNA is 20 nucleotides, the ssPNA is 10 PNA units and the ssPNA binds the CRISPR RNA 0, 5, 10, 15 or 20 nucleotides distal to the PAM site.

40. The pharmaceutical composition of paragraph 39, wherein the ssPNA oligomer is effective to reduce or eliminate Cas localization and/or activity at the off-target site, preferably while activity at the similar or related on-target site is reduced to a lesser extent or preferably is not reduced.

41. The pharmaceutical composition of any one of paragraphs 36-40 further including an effective amount of the Cas enzyme of the CRISPR/Cas, or a nucleic acid expression construct encoding the same.

42. The pharmaceutical composition of paragraph 41, wherein the Cas enzyme has double-strand cutting nuclease activity.

43. The pharmaceutical composition of paragraph 41, wherein the Cas enzyme has only single strand cutting activity (“nickase”).

44. The pharmaceutical composition of paragraph 41, wherein the Cas enzyme has no strand cutting activity (“dead”).

45. The pharmaceutical composition of any one of paragraphs 35-44 including an effective amount of one or more additional ssPNA oligomers directed to different CRISPR/Cas target sites.

46. The pharmaceutical composition of paragraph 45, wherein the number of additional ssPNA oligomers directed to different CRISPR/Cas target sites are between 1 and 1,000 inclusive, or any subrange or specific integer number there between.

47. A method of reducing Cas localization and/or activity at a desired target site in the genome of a population of cells including contacting the cells with an effective amount of the ssPNA oligomer of any one of paragraphs 1-30 or the pharmaceutical composition of paragraphs 35-46.

48. The method of paragraph 47, wherein the ssPNA oligomer is hybridized to a spacer-sequence containing CRISPR RNA.

49. The method of paragraph 48, wherein the method includes a step of annealing the ssPNA oligomer to a spacer-sequence containing CRISPR RNA prior to contacting the cells with an effective amount of the ssPNA oligomer.

50. The method of any one of paragraphs 47-49, wherein the desired target site is an on-target site, and Cas localization and/or activity is reduced or eliminated at the desired on-target target site.

51. The method of any one of paragraphs 47-49, wherein the desired target site is an off-target site, and Cas localization and/or activity is reduced or eliminated at the desired off-target target site.

52. The method of paragraph 51, wherein Cas localization and/or activity is reduced or eliminated at the desired off-target target site but is not eliminated at the on-target site.

53. The method of paragraph 52, wherein Cas localization and/or activity is reduced to a lesser extent relative to the on-target site.

54. The method of paragraph 52, wherein Cas localization and/or activity is not reduced at the on-target site.

55. The method of any one of paragraphs 51-54, wherein the desired target site includes at least 20, 30, 40, 50, 60, 70, 75, 80, 85, 90, or 95% sequence identity to another CRISPR/Cas target site or the reverse complement thereof.

56. The method of paragraph 55, wherein the desired target site includes less than 100, 95, 90, 85, 80, 75, 70, 60, 50, 40, 30, or 20% sequence identity to another CRISPR/Cas target site or the reverse complement thereof.

57. The method of any one of paragraphs 52-56, wherein the desired target site and the CRISPR/Cas target site or its reverse complement share at least between 5-25, or any subrange or specific integer number thereof, nucleobases in common, optionally wherein the nucleobases in common are contiguous.

58. The method of any one of paragraphs 47-57 further including contacting the cells with a gRNA that targets the desired target site, or a nucleic acid expression construct encoding the same.

59. The method of any one of paragraphs 47-58 further including contacting the cells with a Cas enzyme, or a nucleic acid expression construct encoding the same.

60. The method of any one of paragraphs 47-59, further including contacting the cells with a donor oligonucleotide to recombine into the genome adjacent to the desired target site.

61. The method of any one of paragraphs 47-60, wherein the cells are contacted in vitro or ex vivo.

62. The method of any one of paragraphs 47-60, wherein the cells are contacted in vivo.

63. The method of any one of paragraphs 47-62, wherein the Cas enzyme has double-strand cutting nuclease activity or single-strand cutting activity, and optionally further including contacting the cells with a donor oligonucleotide.

64. The method of paragraph 63, including editing the genome at or adjacent to the desired target site.

65. The method of paragraph 64, wherein the editing includes introducing a donor oligonucleotide and/or mutating existing nucleotides at or adjacent to the target site.

66. The method of paragraph 65, wherein the donor oligonucleotide or mutation corrects a disease-causing nucleic acid sequence.

67. The method of any one of paragraphs 59-62, wherein the Cas enzyme does not have nuclease cutting activity.

68. The method of paragraph 67, wherein the Cas enzyme is a fusion protein further including a heterologous polypeptide.

69. The method of paragraph 68, wherein the heterologous polypeptide including a DNA binding domain, deaminase domain, or a protein-binding domain.

70. The method of any of paragraphs 67-69, wherein the Cas enzyme modulates gene regulation, epigenetic editing, chromatin engineering, or imaging.

71. The method of any one of paragraphs 47-70, wherein the ssPNA oligomer is present at a ratio of ssPNA:sgRNA at a molar ratio of at least 1:1.

72. The method of any one of paragraphs 47-71, wherein the ssPNA oligomer is present in an amount between 30 pmol and 250 pmol, inclusive.

73. The method of any one of paragraphs 47-72, wherein the ssPNA oligomer is present in an amount between 50 pmol and 150 pmol, inclusive.

74. The method of any one of paragraphs 47-73, wherein the ssPNA oligomer is present in an amount of 150 pmol.

75. A method of reducing Cas localization and/or activity at a desired target site in the genome of a population of cells including contacting the cells with an effective amount of an ssPNA oligomer including a nucleic acid sequence of any one of the sequences set forth in Table 3 or Table 3A.

76. The method of paragraph 75, wherein the ssPNA is annealed to a gRNA.

77. The method of paragraph 75 or 76, including one or more steps for hybridizing the ssPNA to a gRNA prior to contacting the cells.

78. A CRISPR RNA/PNA duplex, including a spacer sequence-containing CRISPR RNA,
wherein the spacer sequence is complimentary to a protospacer sequence of a target genomic DNA,
wherein the spacer sequence includes from 15 to 25 contiguous nucleobases, inclusive, or any subrange of specific integers there between,
wherein the PNA includes 5-25 contiguous nucleobases inclusive, or any subrange of specific integers there between,
wherein at least 50% up to 100% of the PNA nucleobases are complimentary to the nucleotides in the spacer sequence and
wherein the at least 50% up to 100% of the PNA nucleobases are hybridized to the complimentary nucleotides in the spacer sequence.

79. The CRISPR RNA/PNA duplex of paragraph 78, wherein the PNA is hybridized to the spacer sequence by Watson-Crick binding only.

80. The CRISPR RNA/PNA duplex of paragraphs 78 or 79, wherein the PNA is hybridized to 1, 2, 3, 41 5, 6, 7, 8, 9, 10, 11, 12, 13 14, 15, 16, 17, 18, 19, 20, 21, 22, 23, 24 or 25 nucleotides of the CRISPR RNA spacer sequence.

81. The CRISPR RNA/PNA duplex of any one of paragraphs 78 to 80, wherein the hybridization includes 0, 1, 2, 3, 4, 5 or more mismatches, gaps, and/or insertions.

82. The CRISPR RNA/PNA duplex of any one of paragraphs 78 to 81, wherein the PNA is hybridized to all the nucleotides in the spacer sequence.

83. The CRISPR RNA/PNA duplex of any one of paragraphs 78 to 82, wherein the CRISPR RNA is or includes a crRNA.

84. The CRISPR RNA/PNA duplex of any one of paragraphs 78 to 82, wherein the CRISPR RNA includes a tracrRNA.

85. The CRISPR RNA/PNA duplex of any one of paragraphs 78 to 82, wherein the CRISPR RNA is or includes a guide RNA (gRNA) or single guide RNA (sgRNA).

86. The CRISPR RNA/PNA duplex of any one of paragraphs 78 to 85, wherein the PNA is or includes 10 or 20 nucleic acids in length.

87. The CRISPR RNA/PNA duplex of any one of paragraphs 78 to 86, wherein the CRISPR RNA/PNA duplex exhibits reduced binding to the protospacer sequence, and optionally cannot bind to the protospacer sequence as compared to a corresponding CRISPR RNA in the absence of the PNA.

88. The CRISPR RNA/PNA duplex of any one of paragraphs 78 to 81 or 83-87, wherein the PNA includes at least an integer number between 1 and 25, inclusive, of bases between −20 and −1 of the target site's protospacer sequence, wherein −1 is the last nucleobase of the protospacer and +1 is the first nucleobase of the PAM sequence.

89. The CRISPR RNA/PNA duplex of any one of paragraphs 78 to 88, wherein some or all of the PNA residues are modified at the gamma position.

90. The CRISPR RNA/PNA duplex of paragraph 89, wherein the modification is diethylene glycol, or substitution with an amino acid side chain, optionally selected from alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof.

91. The CRISPR RNA/PNA duplex of any one of paragraphs 78 to 90, wherein none of the PNA residues are modified at the gamma position.

92. The CRISPR RNA/PNA duplex of any one of paragraphs 78 to 91, wherein the PNA includes one or more heterocyclic bases selected from uracil, thymine, cytosine, adenine, guanine, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives.

93. The CRISPR RNA/PNA duplex of paragraph 92, wherein the heterocyclic bases are selected from thymine, cytosine, adenine, and guanine.

94. The CRISPR RNA/PNA duplex of any one of paragraphs 78-93, wherein the PNA includes one or more positively charged moieties.

95. The CRISPR RNA/PNA duplex of paragraph 94, wherein the positively charge moieties are selected from lysine and arginine.

96. The CRISPR RNA/PNA duplex of paragraph 94 or 95, wherein one or more positively charged moieties are present at the N-terminus, C-terminus, or both.

97. The CRISPR RNA/PNA duplex of any one of paragraphs 78-96, including at least 20 PNA residues and one, two or three lysines at each of the N-terminus, or the and C-terminus, or both the N-terminus and C-terminus.

98. The CRISPR RNA/PNA duplex of any one of paragraphs 78 to 97, wherein the PNA includes a nucleic acid sequence of any one of the sequences set forth in Table 3 or Table 3A.

99. A pharmaceutical composition including an effective amount of the CRISPR RNA/PNA duplex of any one of paragraphs 78-98, to reduce activity of the CRISPR/Cas at the target site.

100. A method of reducing Cas localization and/or activity at a desired target site in the genome of a population of cells including contacting the cells with an effective amount of the CRISPR RNA/PNA duplex of any one of paragraphs 78-98 or the pharmaceutical composition of paragraph 99.

101. The method of paragraph 100, wherein the desired target site is an on-target site, and Cas localization and/or activity is reduced or eliminated at the desired on-target target site.

102. The method of paragraph 100, wherein the desired target site is an off-target site, and Cas localization and/or activity is reduced or eliminated at the desired off-target target site.

103. The method of paragraph 100, wherein Cas localization and/or activity is reduced or eliminated at the desired off-target target site but is not eliminated at the on-target site.

104. The method of paragraph 103, wherein Cas localization and/or activity is reduced to a lesser extent relative to the on-target site.

105. The method of paragraph 103, wherein Cas localization and/or activity is not reduced at the on-target site.

106. The method of any one of paragraphs 100-105, wherein the desired target site includes at least 20, 30, 40, 50, 60, 70, 75, 80, 85, 90, or 95% sequence identity to another CRISPR/Cas target site or the reverse complement thereof.

107. The method of paragraph 100-105, wherein the desired target site includes less than 100, 95, 90, 85, 80, 75, 70, 60, 50, 40, 30, or 20% sequence identity to another CRISPR/Cas target site or the reverse complement thereof.

108. The method of any one of paragraphs 106-107, wherein the desired target site and the other CRISPR/Cas target site or its reverse complement share at least between 5-25, or any subrange or specific integer number thereof, nucleobases in common, optionally wherein the nucleobases in common are contiguous.

109. The method of any one of paragraphs 100-108, further including contacting the cells with a CRISPR RNA that targets the desired target site, or a nucleic acid expression construct encoding the same.

110. The method of any one of paragraphs 100-109, further including contacting the cells with a Cas enzyme, or a nucleic acid expression construct encoding the same.

111. The method of any one of paragraphs 100-110, further including contacting the cells with a donor oligonucleotide to recombine into the genome adjacent to the desired target site.

112. The method of any one of paragraphs 100-111, wherein the cells are contacted in vitro or ex vivo.

113. The method of any one of paragraphs 100-111, wherein the cells are contacted in vivo.

114. The method of any one of paragraphs 100-113, wherein the Cas enzyme has double-strand cutting nuclease activity or single-strand cutting activity, and optionally further including contacting the cells with a donor oligonucleotide.

115. The method of paragraph 114, including editing the genome at or adjacent to the desired target site.

116. The method of paragraph 115, wherein the editing includes introducing a donor oligonucleotide and/or mutating existing nucleotides at or adjacent to the target site.

117. The method of paragraph 116, wherein the donor oligonucleotide or mutation corrects a disease-causing nucleic acid sequence.

118. The method of any one of paragraphs 110-113, wherein the Cas enzyme does not have nuclease cutting activity.

119. The method of paragraph 118, wherein the Cas enzyme is a fusion protein further including a heterologous polypeptide.

120. The method of paragraph 119, wherein the heterologous polypeptide includes a DNA binding domain, deaminase domain, or a protein-binding domain.

121. The method of any one of paragraphs 118-120, wherein the Cas enzyme modulates gene regulation, epigenetic editing, chromatin engineering, or imaging.

122. The method of any one of paragraphs 100-121, wherein the CRISPR RNA/PNA duplex is in an amount between 30 pmol and 250 pmol, inclusive.

123. The method of any one of paragraphs 100-122, wherein the CRISPR RNA/PNA duplex is in an amount between 50 pmol and 150 pmol, inclusive.

124. The method of any one of paragraphs 100-123, wherein the CRISPR RNA/PNA duplex is in an amount of 150 pmol.

125. Any of the foregoing paragraphs wherein the PNA further includes a non-PNA molecule conjugated or otherwise attached thereto, optionally wherein the non-PNA molecule is a targeting moiety that increases accumulation of the PNA in a particular tissue or cell types, optionally wherein the targeting moiety is a sugar.

126. A composition for spatiotemporal control of CRISPR Cas, including
(a) a single stranded anti-spacer peptide nucleic acid oligomer (ssPNA),
wherein the ssPNA includes a nucleobase sequence that hybridizes to part of, or all of the spacer sequence of a spacer-sequence containing CRISPR RNA that binds to a protospacer sequence of a CRISPR/Cas target site in a double stranded DNA.

127. The composition of claim 126 further including
(b) the spacer sequence-containing CRISPR RNA,
wherein at least 50%, up to 100% of the nucleotides within the spacer sequence are complimentary to the nucleobases in the ssPNA.

128. The composition of claim 127,
wherein the at least 50% up to 100% of the PNA nucleobases are hybridized to the complimentary nucleotides in the spacer sequence.

129. The composition of any one of paragraphs 126-128, wherein the ssPNA hybridizes to the CRISPR RNA by Watson-Crick binding only.

130. The composition of any one of paragraphs 126-129,
wherein the ssPNA includes 5-25 contiguous nucleobases inclusive, or any subrange of specific integers there between,

131. The composition of any one of paragraphs 126-130,wherein the spacer sequence of the spacer-sequence containing CRISPR RNA includes from 15 to 25 contiguous nucleobases, inclusive, or any subrange of specific integers there between.

132. The composition of any one of paragraphs 126-131, wherein
(i) the nucleobase sequence of the ssPNA hybridizes to any integer number between 1-25, 5-25, 10-25, 15-25, 1-20, 5-20, 5-15, or 8-12 nucleotides of the spacer sequence of the CRISPR RNA; and/or
(ii) the nucleobase sequence of the ssPNA hybridizes to between about 25% and about 100%, inclusive, of the nucleotides of the spacer sequence of the CRISPR RNA.

133. The composition of any one of paragraphs 126-132, wherein the hybridization includes 0, 1, 2, 3, 4, 5 or more mismatches, gaps, and/or insertions

134. The composition of any one of paragraphs 126-133, wherein the ssPNA does not form a PNA:DNA:DNA triplex, or a PNA:DNA:PNA triplex with the CRISPR RNA or at the CRISPR/Cas target site.

135. The composition of any one of paragraphs 126-134, wherein the nucleobase sequence includes
(i) part of the protospacer sequence; or
(ii) the entire protospacer sequence.

136. The composition of any one of paragraphs 126-135, wherein, the CRISPR RNA is or includes
(i) a crRNA; or
(ii) a tracrRNA; or
(iii) a guide RNA (gRNA); or
(iv) a single guide RNA (sgRNA).

137. The composition of any one of paragraphs 126-136, wherein the ssPNA includes between about 5 and about 50 PNA residues in length, more preferably about 8 to about 30 residues, or about 10 to about 20 residues, or about 8 to about 12 residues, or about 10 residues in length.

138. The composition of any one of paragraphs 126-137, wherein the CRISPR RNA that targets the CRISPR/Cas target site exhibits reduced binding, and optionally cannot bind to the protospacer sequence, when the ssPNA is present.

139. The composition of any one of paragraphs 126-137, wherein a Cas enzyme that binds to the CRISPR RNA that targets the CRISPR/Cas target site
(i) exhibits reduced binding to the CRISPR/Cas target site; or
(ii) cannot bind to the CRISPR/Cas target site, when the ssPNA is present.

140. The composition of any one of paragraphs 126-139, wherein the ssPNA includes at least an integer number between 5 and 40, inclusive, of bases between −20 and −1 of the target site's protospacer sequence, wherein −1 is the last nucleobase of the protospacer and +1 is the first nucleobase of the protospacer adjacent motif (PAM) of the target site.

141. The composition of any one of paragraphs 126-140, wherein some of, or all of the PNA residues are modified at the gamma position,
optionally wherein the modification is diethylene glycol, or substitution with an amino acid side chain,
optionally wherein the amino acid side chain is selected from alanine, serine, threonine, cysteine, valine, leucine, isoleucine, methionine, proline, phenylalanine, tyrosine, aspartic acid, glutamic acid, asparagine, glutamine, histidine, lysine, and arginine, or the derivatives thereof.

142. The composition of any one of paragraphs 126-141, wherein the ssPNA includes heterocyclic bases selected from uracil, thymine, cytosine, adenine, guanine, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives.

143. The composition of any one of paragraphs 126-142, wherein the ssPNA includes one or more positively charged moieties,
optionally wherein
(i) the one or more positively charged moieties are selected from lysine and arginine; and/or
(ii) the one or more positively charged moieties are present at the N-terminus, C-terminus.

144. The composition of any one of paragraphs 126-143, wherein the ssPNA includes at least 20 PNA residues, and includes one, two or three lysines at the N-terminus, or the C-terminus, or both the N-terminus and C-terminus.

145. The composition of any one of paragraphs 126-144, wherein the ssPNA includes a nucleobase sequence of any one of the sequences set forth in Table 3 or Table 3A.

146. The composition of any one of paragraphs 126-145, further including
(c) a Cas enzyme, or a nucleic acid expression construct encoding a Cas enzyme.

147. The composition of paragraphs 146, wherein the Cas enzyme is complexed with the CRISPR RNA.

148. The composition of paragraphs 146 or 147, wherein the Cas enzyme is selected from spCas9, FnCas9, SaCas9, NmCas9, St1Cas9, St3Cas9, AsCPf1, LbCpf1, and Cas13. 149. The composition of any one of paragraphs 146-148, wherein the Cas enzyme
(i) has double-strand cutting nuclease activity; or
(ii) has single strand cutting (“nickase”) activity; or
(iii) has no strand cutting (“dead”) activity;
optionally wherein the Cas enzyme is a fusion protein further including a heterologous polypeptide,
optionally wherein the heterologous polypeptide includes one or more of a DNA binding domain, a deaminase domain, and a protein-binding domain.

150. The composition of any one of paragraphs 126-149 packaged in a microparticle or nanoparticle, optionally wherein the particle is a liposome or polymeric particle.

151. The composition of any one of paragraphs 126-150, wherein
(i) the target sequence of the spacer-sequence containing CRISPR RNA is 15-25 nucleotides, or any subrange of specific integer there between; and
(ii) the ssPNA is 5-20 PNA residues or any subrange of specific integer there
   between,
wherein the ssPNA binds the spacer sequence at a site that is at least 1, 2, 3, 4, 5, 6, 7, 8, 9, 10, 11, 12, 13, 14, 15, 16, 17, 18, 19, or 20 nucleotides distal to a nucleotide that forms the protospacer adjacent motif (PAM) of the CRISPR/Cas target site.

152. The composition of any one of paragraphs 126-151, wherein
(i) the target sequence of the spacer-sequence containing CRISPR RNA is 20 nucleotides; and
(ii) the ssPNA includes 10 PNA residues; and
(iii) the ssPNA binds the CRISPR RNA at a site that is 0, 5, 10, 15 or 20 nucleotides distal to a nucleotide that forms the protospacer adjacent motif (PAM) of the CRISPR/Cas target site.

153. A pharmaceutical composition including an effective amount of the composition of any one of paragraphs 126-152.

154. The pharmaceutical composition of paragraph 153, wherein the composition is in an amount effective to reduce or prevent the activity of a CRISPR/Cas complex at the CRISPR/Cas target site.

155. The pharmaceutical composition of paragraph 153, wherein the composition is in an amount effective to reduce activity of a CRISPR/Cas complex at an off-target site of the CRISPR/Cas target site.

156. The pharmaceutical composition of paragraph 155, wherein the composition is in an amount effective to reduce or eliminate Cas localization and/or activity at the off-target site, preferably while activity at the similar or related on-target site is reduced to a lesser extent or preferably is not reduced.

157. The pharmaceutical composition of any one of paragraphs 153-156 including an effective amount of one or more additional single stranded anti-spacer peptide nucleic acid oligomers directed to one or more different CRISPR/Cas target sites.

158. The pharmaceutical composition of paragraph 157, wherein the number of additional single stranded anti-spacer peptide nucleic acid oligomers directed to one or more different CRISPR/Cas target sites is between 1 and 1,000 inclusive, or any subrange or specific integer number there between.

159. A method of reducing Cas localization and/or activity at a desired target site in the genome of a population of cells including contacting the cells with an effective amount of the composition of any one of paragraphs 126-152, or the pharmaceutical composition of any one of paragraphs 153-158.

160. The method of paragraph 159, wherein the ssPNA is hybridized to a spacer-sequence containing CRISPR RNA.

161. The method of paragraph 159 or 160, wherein the method includes a step of annealing the ssPNA to a spacer-sequence containing CRISPR RNA prior to contacting the cells with the composition or the pharmaceutical composition.

162. The method of paragraph 159 or 160 or 161, wherein the desired target site is an on-target site, and Cas localization and/or activity is reduced or eliminated at the desired on-target target site.

163. The method of any one of paragraphs 159 to 162, wherein the desired target site is an off-target site, and Cas localization and/or activity is reduced or eliminated at the desired off-target target site.

164. The method of any one of paragraphs 159 to 163, wherein Cas localization and/or activity is reduced or eliminated at the desired off-target target site but is not eliminated at the on-target site.

165. The method of any one of paragraphs 159 to 163, wherein Cas localization and/or activity is reduced to a lesser extent relative to the on-target site, or wherein Cas localization and/or activity is not reduced at the on-target site.

166. The method of any one of paragraphs 159 to 165, wherein the desired target site includes at least 20, 30, 40, 50, 60, 70, 75, 80, 85, 90, or 95% sequence identity to another CRISPR/Cas target site or the reverse complement thereof.

167. The method of paragraph 166, wherein the desired target site includes less than 100, 95, 90, 85, 80, 75, 70, 60, 50, 40, 30, or 20% sequence identity to another CRISPR/Cas target site or the reverse complement thereof.

168. The method of any one of paragraphs 159 to 167, wherein the desired target site and the CRISPR/Cas target site or its reverse complement share at least between 5-25, or any subrange or specific integer number thereof, nucleotides in common, optionally wherein the nucleotides in common are contiguous.

169. The method of any one of paragraphs 159 to 168, further including contacting the cells with a spacer sequence containing CRISPR RNA that targets the desired CRISPR/Cas target site, or a nucleic acid expression construct encoding the same.

170. The method of any one of paragraphs 159 to 169 further including contacting the cells with a Cas enzyme, or a nucleic acid expression construct encoding the same.

171. The method of paragraph 170, wherein the Cas enzyme is a fusion protein further including a heterologous polypeptide, optionally wherein the heterologous polypeptide includes a DNA binding domain, deaminase domain, or a protein-binding domain.

172. The method of paragraph 170, or 171, wherein the Cas enzyme modulates gene regulation, epigenetic editing, chromatin engineering, or imaging.

173. The method of any one of paragraphs 159 to 172, further including contacting the cells with a donor oligonucleotide to recombine into the genome adjacent to the desired CRISPR/Cas target site.

174. The method of any one of paragraphs 159 to 173, wherein the cells are contacted in vitro or ex vivo.

175. The method of any one of paragraphs 159 to 174, wherein the cells are contacted in vivo.

176. The method of any one of paragraphs 159 to 175, including editing the genome of the cells at or adjacent to the desired CRISPR/Cas target site.

177. The method of paragraph 176, wherein the editing includes introducing a donor oligonucleotide and/or mutating existing nucleotides at or adjacent to the CRISPR/Cas target site.

178. The method of paragraph 177, wherein the donor oligonucleotide or mutation corrects a disease-causing nucleic acid sequence.

179. The method of any one of paragraphs 159 to 178, including modulating gene regulation, epigenetic editing, chromatin engineering, or imaging.

180. The method of any one of paragraphs 159 to 179, wherein the ssPNA is present at a ratio of ssPNA:CRISPR RNA at a molar ratio of at least 1:1. 181. The method of any one of paragraphs 159 to 180, wherein the ssPNA is present in an amount between 30 pmol and 250 pmol, inclusive, optionally wherein the ssPNA is present in an amount between 50 pmol and 150 pmol, inclusive.

182. The method of any one of paragraphs 159 to 181, wherein the ssPNA is present in an amount of 150 pmol.

183. A method of reducing Cas localization and/or activity at a desired target site in the genome of a population of cells including contacting the cells with an effective amount of an ssPNA including a nucleic acid sequence of any one of the sequences set forth in Table 3 or Table 3A.

The present invention will be further understood by reference to the following non-limiting examples.

### Examples

### Example 1: Antispacer PNAs can Block CRISPR/Cas Gene Editing

PNAs are synthetic chimeric oligonucleotides modified to feature a neutrally charged polyamide backbone (Egholm, et al., Nature, 365, 566 (1993); Nielsen, et al., Bioconjugate Chemistry, 5, 3-7 (1994)). Due to minimized repulsive forces between polymer backbones these molecules bind complementary DNA and RNA sequences with remarkably high affinity and specificity (Pellestor and Paulasova, European Journal Of Human Genetics, 12, 694 (2004)). For these reasons, PNAs have been widely employed as antisense or antigene molecules for applications such as gene targeting, microRNA modulation, and mRNA-binding protein identification (Zeng, et al., Nature Protocols, 1, 920-927 (2006). Fabbri, et al., ChemMedChem, 6, 2192-2202 (2011); Peter, Current Pharmaceutical Design, 16, 3118-3123 (2010); and Economos, et al., Molecules, 25 (2020)). Meanwhile, CRISPR-Cas9 systems are known to target double-stranded DNA (dsDNA) sequences in an interaction that is dependent on protospacer adjacent motif (PAM) sequence recognition followed by base-pairing between a target DNA strand and a complementary spacer sequence coded by an internalized gRNA (R-loop formation, FIG. 1A) (Sternberg, et al., Nature, 507, 62-67 (2014); Anders, et al., Nature, 513, 569-573 (2014)).

### Materials and Methods

### PNA Synthesis and Purification: Automated PNA Synthesis

PNA oligomers were synthesized using a Biotage Initiator+ Alstra microwave peptide synthesizer. The desired sequence was assembled automatically on 10% L-lysine loaded Rink Amide ChemMatrix resin (Sigma Aldrich, 727768-5G) using standard Fmoc chemistry. All Fmoc-aeg-PNA monomers were purchased from PNA BIO INC, Thousand Oaks. The oligomers were cleaved from the resin at room temperature using a trifluoroacetic acid (TFA): water: triisopropylsilane: (38:1:1) cocktail solution (60 min×1). The crude PNAs were precipitated with cold ether, purified, and characterized by reverse phase-high performance liquid chromatography (RP-HPLC) (5-95% ACN/Water/0.1% TFA gradient) and MALDI-TOF spectroscopy (MALDI-TOF-MS Shimadzu AXIMA Confidence), respectively.

### PNA Synthesis and Purification: Manual PNA Synthesis

TAMRA dye-labeled PNA oligomers were synthesized manually on 10% L-lysine loaded solid support (MBHA (4-methylbenzhydrylamine) resin, Peptides International, RMB-1045-PI) using standard Boc chemistry procedures. All Boc-aeg-PNA monomers were purchased from ASM Research Chemicals GmbH (Hannover, Germany). Kaiser tests were performed to ensure complete deprotection and coupling during each cycle. The oligomers were cleaved from the resin using a m-cresol: thioanisole: trifluoromethanesulfonic acid (TFMSA): trifluoroacetic acid (TFA) (1:1:2:6) cocktail solution (30 min×2). The resulting mixtures were combined and the crude PNAs were precipitated with cold ether, purified, and characterized by reverse phase-high performance liquid chromatography (RP-HPLC) (5-95% ACN/Water/0.1% TFA gradient) and MALDI-TOF spectroscopy (MALDI-TOF-MS Shimadzu AXIMA Confidence), respectively.

All PNA stock solutions (automated and manual) were prepared using nanopure water, and the concentrations were determined using a Thermo Scientific™ NanoDrop™ OneC microvolume spectrophotometer. The following extinction coefficients were used: 13,700 M-lcm-1 (A), 6,600 M-1 cm-1 (C), 11,700 M-lcm-1 (G), and 8,600 M-lcm-1 (T).

20mer PNAs were all synthesized with three L-lysine (K) residues on either C- or N-terminus to facilitate solubility. 10mer PNAs were synthesized with a single L-lysine (K) residue on either terminus. HPLC instrument set up included: Waters 2998 Photodiode Array Detector, Waters 2545 Quaternary Gradient Module, Waters 2707 Autosampler.

In some cases, purified, synthesized, and characterized PNAs were acquired directly from PNA BIO INC. Refer to Tables 3/3A for all PNA sequences and sources used in this study.

TABLE 1
Guide RNAs sequences

| sgRNA | Protospacer Sequence | PAM | Reference |
|---|---|---|---|
| BFP | GCTGAAGCACTGCACGCCAT | GGG | Richardson, C. D., Ray, G. J., |
| sgRNA | (SEQ ID NO: 1) | | DeWitt, M. A., Curie, G. L. & Corn, |
| | | | J. E. Enhancing homology-directed |
| | | | genome editing by catalytically |
| | | | active and inactive CRISPR-Cas9 |
| | | | using asymmetric donor DNA. Nat. |
| | | | Biotechnol. 34, 339-344 (2016). |
| HBB R-01 | GTGAACGTGGATGAAGTTGG | TGG | Cradick, T. J., Fine, E. J., Antico, C. |
| sgRNA | (SEQ ID NO: 2) | | J. & Bao, G. CRISPR/Cas9 systems |
| | | | targeting ß-globin and CCR5 genes |
| | | | have substantial off-target activity. |
| | | | Nucleic Acids Res. 41, 9584-9592 |
| | | | (2013). |

Guide RNAs were ordered as 100nt single guide RNAs (sgRNAs) from IDT with the specified protospacer sequence.

TABLE 2
Target Sequences

| Target | Target Sequence (100 bp segment) |
|---|---|
| BFP | CTGCCGGTGCCCTGGCCCACCCTCGTGACCACCCTGACCCATGGCGTG |
| | CAGTGCTTCAGCCGCTACCCCGACCACATGAAGCAGCACGACTTCTTC |
| | AAGT (SEQ ID NO: 3) |
| HBB | TCCTGAGGAGAAGTCTGCCGTTACTGCCCTGTGGGGCAAGGTGAACGT |
| | GGATGAAGTTGGTGGTGAGGCCCTGGGCAGGTTGGTATCAAGGTTACA |
| | AGAC (SEQ ID NO: 4) |

Cas9 target sequences are underlined with protospacer in italics and PAM sequence in bold.

TABLE 3
PNA sequences

| PNA | Sequence |
|---|---|
| BEP Anti-Protospacer PNA | KKK-ATGGCGTGCAGTGCTTCAGC-KKK (SEQ ID NO: 6) |
| (Also BFP Bottom −20 to −1) | |
| GFP non-Targeting PNA | KKK-GGGTTAAGGCAATAGCAATA-KKK (SEQ ID NO: 7) |
| HBB PNA | KKK-GGCCTCACCACCAACTGCAT-KKK (SEQ ID NO: 8) |
| BFP Top −40 to −21 | KKK-CGCTACCCCGACCACATGAA-KKK (SEQ ID NO: 9) |
| BFP Top −20 to −11 | KKK-GTGCTTCAGCCGCTACCCCG-KKK (SEQ ID NO: 10) |
| RFP Top −10 to +9 | KKK-ACCCTGACCCATGGCGTGCA-KKK (SEQ ID NO: 11) |
| BFP Top +1 to +20 | KKK-CCTCGTGACCACCCTGACCC-KKK (SEQ ID NO: 12) |
| BFP Bottom −40 to −21 | KKK-TTCATGTGGTCGGGGTAGCG-KKK (SEQ ID NO: 13) |
| BPP Bottom −20 to −11 | KKK-CGGGGTAGCGGCTGAAGCAC-KKK (SEQ ID NO: 14) |
| BFP Bottomn −10 to +9 | KKK-TGCACGCCATGGGTCAGGGT-KKK (SEQ ID NO: 15) |
| BFP Bottom +1 to +20 | KKK-GGGTCAGGGTGGTCACGAGG-KKK (SEQ ID NO: 16) |
| Anti-protospacer ssDNA oligo | ATGGCGTGCAGTGCTTCAGC (unmodified ssDNA oligo from |
| | IDT) (SEQ ID NO: 17) |
| TAMRA BFP Antispacer PNA | TAMRA-KKK-ATGGCGTGCAGTGCTTCAGC-KKK (SEQ ID NO: 29) |
| BFP PAM-proximal PNA | K-ATGGCGTGCA-K (SEQ ID NO: 30) |
| BFP PAM-middle PNA | K-GTGCAGTGCT-K (SEQ ID NO: 31) |
| BFP PAM-distal PNA | K-GTGCTTCAGC-K (SEQ ID NO: 32) |

TABLE 3A
PNA probe nucleic acid sequences

| | Nucleic acid | MW | Extinction | | |
|---|---|---|---|---|---|
| PNA | Sequence | (g/mol) | Coeff (IDT) | Notes | Source |
| BFP Antispacer | ATGGCGTGCAG | 6227.43 | 186000 L/ | 3 Lysine (K) | SNO, NGE- |
| PNA | TGCTTCAGC | | (mole·cm) | on either end | on Biotage |
| | (SEQ ID NO: 17) | | | | Synthesizer |
| TAMRA-BFP | ATGGCGTGCAG | 6694.35 | 186000 L/ | 3 Lysine (K) | KC- |
| Antispacer | TGCTTCAGC | | (mole·cm) | on either end, | Manually |
| PNA | (SEQ ID NO: 17) | | | TAMRA dye | |
| | | | | conjugated at | |
| | | | | N-terminus | |
| HBB R-01 | CCAACTTCATC | 6035.63 | 179400 L/ | 3 Lysine (K) | PNA BIO |
| Antispacer | CACGTTCAC | | (mole·cm) | on either end | |
| PNA | (SEQ ID NO: 33) | | | | |
| BFP Antispacer | ACCCTGACCCA | 6141.51 | 184600 L/ | 3 Lysine (K) | NGE-on |
| −10 to +9 | TGGCGTGCA | | (mole·cm) | on either end | Biotage |
| | (SEQ ID NO: 34) | | | | Synthesizer |
| BFP Antispacer | GTGCTTCAGCC | 6108.36 | 173700 L/ | 3 Lysine (K) | NGE-on |
| −30 to −11 | GCTACCCCG | | (mole·cm) | on either end | Biotage |
| | (SEQ ID NO: 35) | | | | Synthesizer |
| BFP Antispacer | CGCTACCCCGA | 6094.74 | 188100 L/ | 3 Lysine (K) | NGE-on |
| −40 to −21 | CCACATGAA | | (mole·cm) | on either end | Biotage |
| | (SEQ ID NO: 36) | | | | Synthesizer |
| BFP Antispacer | CCTCGTGACCA | 6037.45 | 172000 L/ | 3 Lysine (K) | NGE-on |
| +1 to +20 | CCCTGACCC | | (mole·cm) | on either end | Biotage |
| | (SEQ ID NO: 37) | | | | Synthesizer |
| BFP Cis-spacer | GCTGAAGCACT | 6165.61 | 185600 L/ | 3 Lysine (K) | PNA BIO |
| PNA | GCACGCCAT | | (mole·cm) | on either end | |
| | (SEQ ID NO: 38) | | | | |
| BFP Cis-spacer | CGGGGTAGCGG | 6286.5 | 193100 L/ | 3 Lysine (K) | PNA BIO |
| −30 to −11 | CTGAAGCAC | | (mole·cm) | on either end | |
| | (SEQ ID NO: 39) | | | | |
| BFP Cis-spacer | TTCATGTGGTC | 6298.34 | 191100 L/ | 3 Lysine (K) | PNA BIO |
| −40 to −21 | GGGGTAGCG | | (mole·cm) | on either end | |
| | (SEQ ID NO: 40) | | | | |
| BFP Cisspacer | GGGTCAGGGTG | 6357.41 | 199800 L/ | 3 Lysine (K) | PNA BIO |
| +1 to +20 | GTCACGAGG | | (mole·cm) | on either end | |
| | (SEQ ID NO: 41) | | | | |
| BFP Cis-spacer | TGCACGCCATG | 6252.42 | 188400 L/ | 3 Lysine (K) | PNA BIO |
| −10 to +9 | GGTCAGGGT | | (mole·cm) | on either end | |
| | (SEQ ID NO: 42) | | | | |
| GFP Non- | GGGTTAAGGCA | 6292.92 | 212300 L/ | 3 Lysine (K) | ASR- |
| Targeting PNA | ATAGCAATA | | (mole·cm) | on either end | manually |
| | (SEQ ID NO: 43) | | | | |
| BFP PAMprox | ATGGCGTGCA | 3009.08 | 98600 L/ | 1 Lysine (K) | NGE-on |
| 10 mer PNA | (SEQ ID NO: 44) | | (mole·cm) | on either end | Biotage |
| | | | | | Synthesizer |
| BFP Middle | GTGCAGTGCT | 2999.99 | 92500 L/ | 1 Lysine (K) | NGE-on |
| 10 mer PNA | (SEQ ID NO: 45) | | (mole·cm) | on either end | Biotage |
| | | | | | Synthesizer |
| BFP PAMdist | GTGCTTCAGC | 2959.99 | 89300 L/ | 1 Lysine (K) | NGE-on |
| 10 mer PNA | (SEQ ID NO: 46) | | (mole·cm) | on either end | Biotage |
| | | | | | Synthesizer |
| HBB R-01 | CCAACTGCAT | 2913.18 | 90800 L/ | 1 Lysine (K) | NGE-on |
| PAMprox | (SEQ ID NO: 47) | | (mole·cm) | on either end | Biotage |
| 10 mer PNA | | | | | Synthesizer |
| FANCF | CTTCTGCAGC | 2919.99 | 84500 L/ | 1 Lysine (K) | NGE-on |
| PAMdist 10 mer | (SEQ ID NO: 48) | | (mole·cm) | on either end | Biotage |
| PNA | | | | | Synthesizer |
| ZSCAN | CTTGCCGCAC | 2904.98 | 83200 L/ | 1 Lysine (K) | NGE-on |
| PAMdist 10 mer | (SEQ ID NO: 49) | | (mole·cm) | on either end | Biotage |
| PNA | | | | | Synthesizer |
| VEGFA3 | CTCACTCACC | 2849.08 | 84400 L/ | 1 Lysine (K) | NGE-on |
| PAMdist 10 mer | (SEQ ID NO: 50) | | (mole·cm) | on either end | Biotage |
| PNA | | | | | Synthesizer |
| CCR5 PAMdist | TCCGCTCTAC | 2879.99 | 83700 L/ | 1 Lysine (K) | NGE-on |
| 10 mer PNA | (SEQ ID NO: 51) | | (mole·cm) | on either end | Biotage |
| | | | | | Synthesizer |
| POU5F1 Anti- | CCACCATTAGG | 6093.81 | 190700 L/ | 3 Lysine (K) | NGE-on |
| CRISPRa PNA | CAAACATCC | | (mole·cm) | on either end | Biotage |
| | (SEQ ID NO: 52) | | | | Synthesizer |
| MYOD | GCACGCCCTTT | 5785.27 | 165600 L/ | 3 Lysine (K) | NGE-on |
| Promoter PNA | CCAAACCT | | (mole·cm) | on either end | Biotage |
| | (SEQ ID NO: 53) | | | | Synthesizer |
| EMX1 | GCTCGGACTC | 2994.8 | 88400 L/ | 3 Lysine (K) | NGE-on |
| PAMdist 10 mer | (SEQ ID NO: 54) | | (mole·cm) | on either end | Biotage |
| PNA | | | | | Synthesizer |

All PNAs were synthesized from unmodified PNA monomers and flanked with three lysine (K) residues on either side to improve solubility and promote strand invasion.

### UV Spectroscopy Thermal Melting Analysis

All samples were prepared by mixing a stoichiometric amount of each strand (1 μM) in 10 mM sodium phosphate buffer at pH 7.4 (Table 8). The samples were pre-annealed at 95° C. for 5 mins and gradually cooled to room temperature prior to the melting experiment. UV melting experiments were performed using a Shimadzu UV-3600 Plus UV-Vis-NIR spectrophotometer equipped with a thermoelectrically controlled multicell holder. UV melting spectra were recorded after every 1° C. temperature change by monitoring the absorbance at 260 nm from 20° C. to 110° C. to 20° C., with a heating/cooling ramp rate of 1.0° C./min. The heating and cooling curves were overlapped to confirm reversible denaturation. All spectra were plotted on Origin 2020 and smoothed using a 20-point adjacent averaging algorithm. The first derivative plots of the melting curves were generated to determine the melting temperature for each duplex.

sgRNA Binding Assay

10 pmol of BFP or HBB sgRNA were incubated with or without 10 pmol of purified Cas9 (Sigma) in 20 μL of 1×NEB buffer 3.1 for 15 minutes at room temp. Then, specified doses of a TAMRA dye labeled antispacer BFP ssPNA was added to each reaction and tubes were incubated in thermocycler for 30 minutes at 37C. 5 μL of resulting reactions were run on pre-cast 5% polyacrylamide TBE gels (BIORAD). The gel was washed in 1×TBE buffer and imaged for TAMRA signal using a Gel Doc XR+ gel imager system (BIORAD). Gel was then incubated in 1×SYBR Gold in TBE for 5 minutes, washed in 1×TBE, and imaged for nucleic acid staining on Gel Doc XR+ imager. Resulting images were superimposed using ImageJ.

### Cell Culture

K562 cells (CCL-243, ATCC) were maintained in RPMI-1640 medium supplemented with 10% fetal bovine serum (FBS, Life Technologies). HEK293 cells (CRL-1573, ATCC) were maintained in DMEM medium supplemented with 10% fetal bovine serum (FBS, Life Technologies). U2OS cells (HTB-96, ATCC) were maintained in McCoy's 5A medium supplemented with 10% fetal bovine serum (FBS, Life Technologies). All cell lines were tested and confirmed to be free of mycoplasma infection.

### Cell Lines

dCas9-VPR, CRISPRa

Stable K562-hEF1α-dCas9-VPR line was established by lentiviral transduction using Edit-R purified lentiviral particles (Horizon Discovery) and blasticidin selection (5 μg/mL, A1113903, Gibco). dCas9-VPR integration was confirmed by western blot with CRISPR-Cas9 antibody (7A9-3A3, Novus). dCas9-VPR expressing line was further transduced with Edit-R CRISPRa human POU5F1 sgRNA purified lentiviral particles (VSGH11902, Horizon Discovery, Table 4) and puromycin selection (2 μg/mL, Gibco).

dCas9-p300 Acetyltransferase

Stable K562-pLV-dCas9-p300 line was established from lentivirus generated from pLV-dCas9-p300-P2A-PuroR plasmid (Addgene: #83889) and Invitrogen Virapower packaging plasmids in 293FT cells (Invitrogen, R70007) and puromycin selection (2 μg/mL, Gibco). Lines were further transduced with lentivirus similarly generated with pLV-U6-UbC-eGFP-P2A-Bsr plasmids (Addgene: #83925) containing MYOD promoter targeted sgRNA sequences with blasticidin selection (5 μg/mL, Gibco, A1113903, Table 4).

### K562-BFP

BFP-expressing K562 reporter lines were acquired from the laboratory of Jacob Corn, and feature a lentiviral inserted BFP gene under puromycin selection (Addgene: #111092)(Richardson, et al., Nature Biotechnology, 34, 339-344 (2016)).

Cas9 RNP Formulation and Nucleofections with PNA

For Cas9 RNP assembly, 50 pmol of sgRNA (IDT, Alt-R CRISPR-Cas9 sgRNA) and 45 pmol of SpCas9 (CPO2, PNA BIO) were incubated at room temperature for 10 minutes in a 5 μL reaction in 1×3.1 Buffer (B7203S, NEB). For nucleofections, 1×10⁶ cells, 5 μL Cas9 RNP, and 1 μL of Alt-R Cas9 electroporation enhancer (IDT) were resuspended in 100 μL of Lonza cell line solution (V4XC-2024/V4XC-1024, Lonza) and nucleofected using a Lonza 4D-Nucleofector X unit. Cas9 RNP treated cells were seeded in 2 mL of complete media and incubated for 96 hours prior to harvesting for analysis.

For PNA pre-treatments, 1×10⁶ cells and 1 μL of PNA diluted to desired concentration in water were similarly nucleofected using Lonza cell line kits and 4D-Nucleofector. PNA nucleofected cells were resuspended in 1.25 mL of complete media in 1.5 mL tubes and incubated at 37° C. for 2 hours before Cas9 RNP nucleofection as described above. After Cas9 RNP nucleofection cells were seeded in 2 mL of complete media and incubated for 96 hours prior to harvesting and analysis.

### Flow Cytometry

K562 cells were spun down and resuspended in fresh complete media at 300,000 cells/mL 48 hours prior to analysis. Samples for flow cytometry were fixed in complete media with 1% formaldehyde for 15 minutes before spinning down, resuspending in 300 μL PBS, and filtering through a cell strainer capped tube. Samples were analyzed for BFP and GFP fluorescence using a Cytoflex LX instrument (Beckman Coulter) using PacificBlue and FITC lasers, respectively, and quantification and gating was done using FloJo v10 software. Background fluorescent drop out was determined by measurement of mock nucleofected lines in quadruplicate and subtracted from experimental values to determine reported editing frequency.

### PNA/DNA/Non-Targeting Assay

10⁶ stable BFP-expressing K562 cells were nucleofected (Lonza 4D Nucleofector) with a specified dose of PNA or ssDNA oligo (0-250 pmol). Two hours later, all conditions were nucleofected with 45 pmol of purified Cas9 RNP using a sgRNA targeted to the BFP gene. 72 hours later, cells with BFP fluorescent drop out (via Cas9 indel formation) were quantified by flow cytometry (Beckman Coulter—Cytoflex instrument).

### Statistics

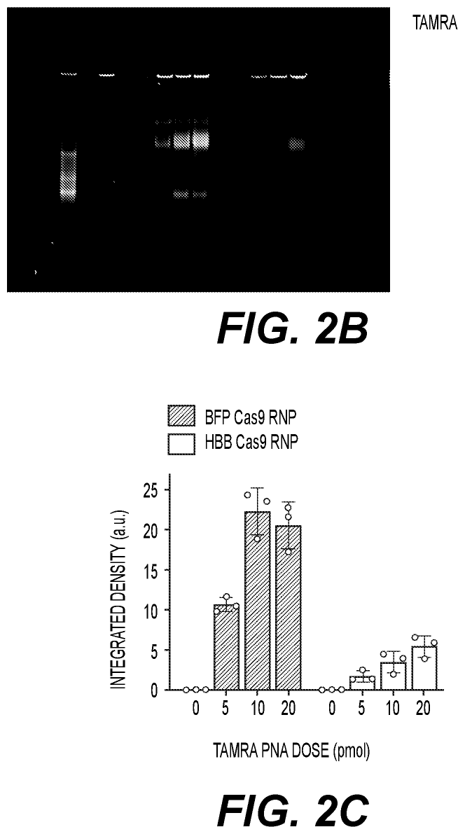

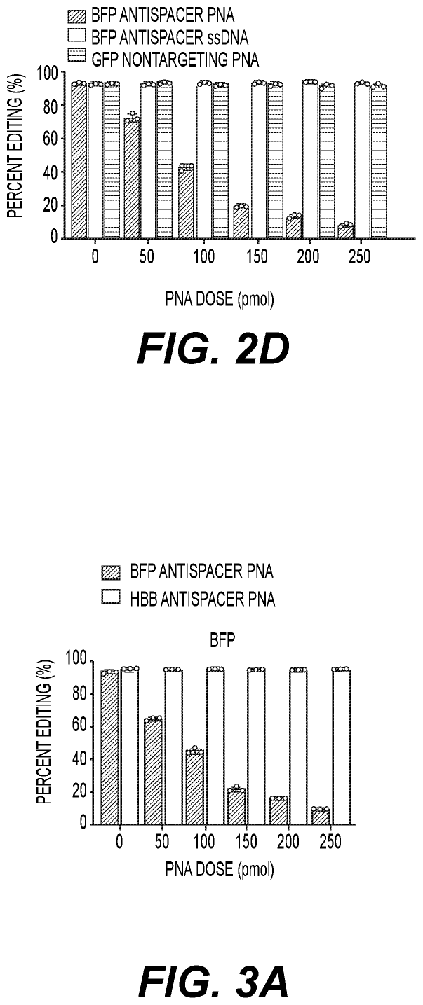

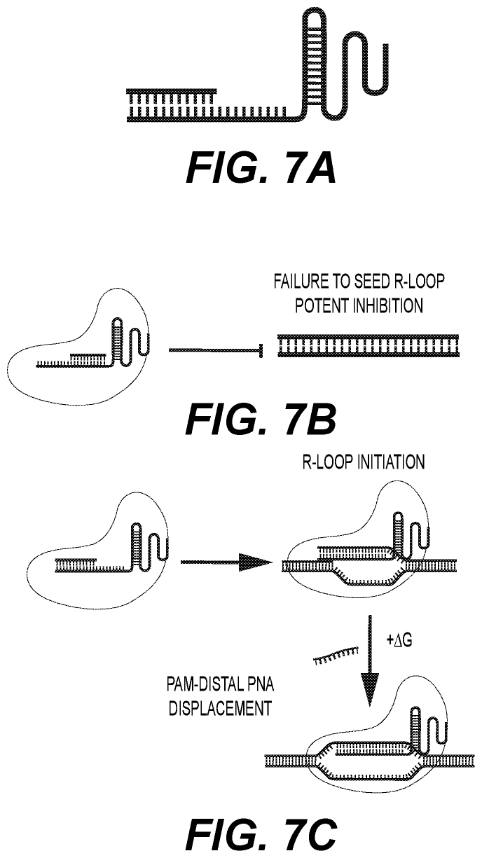

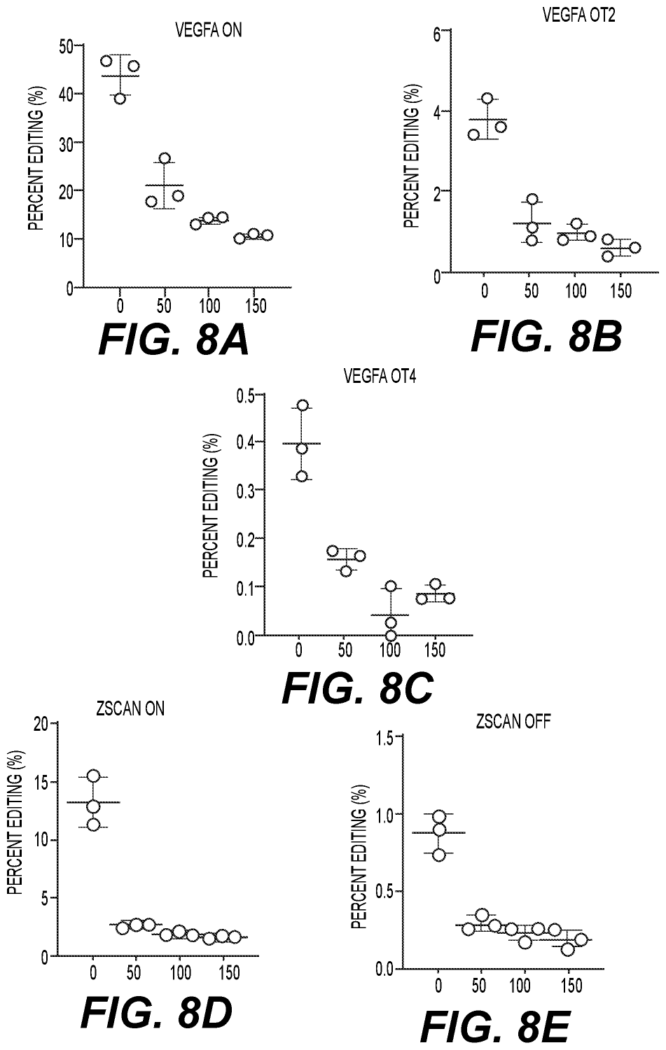

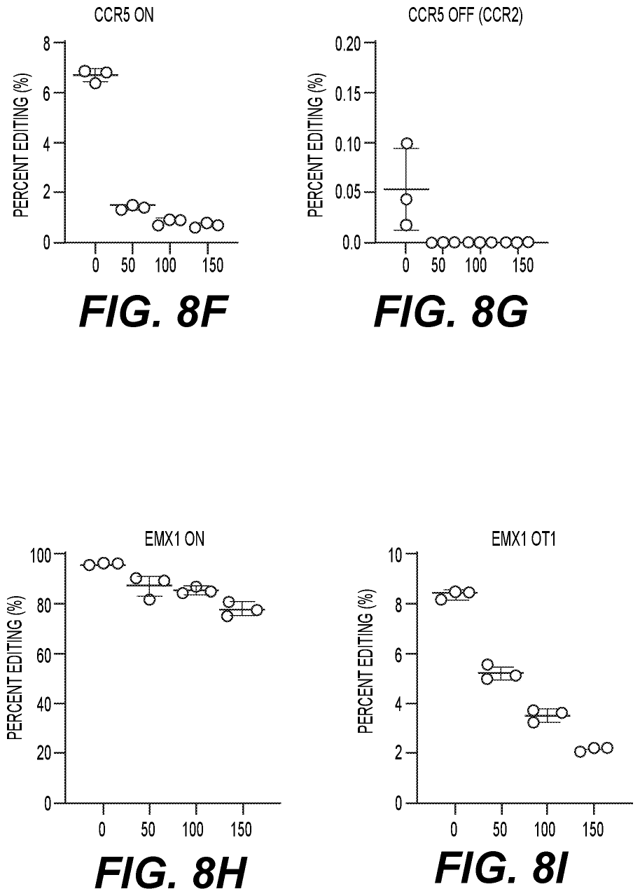

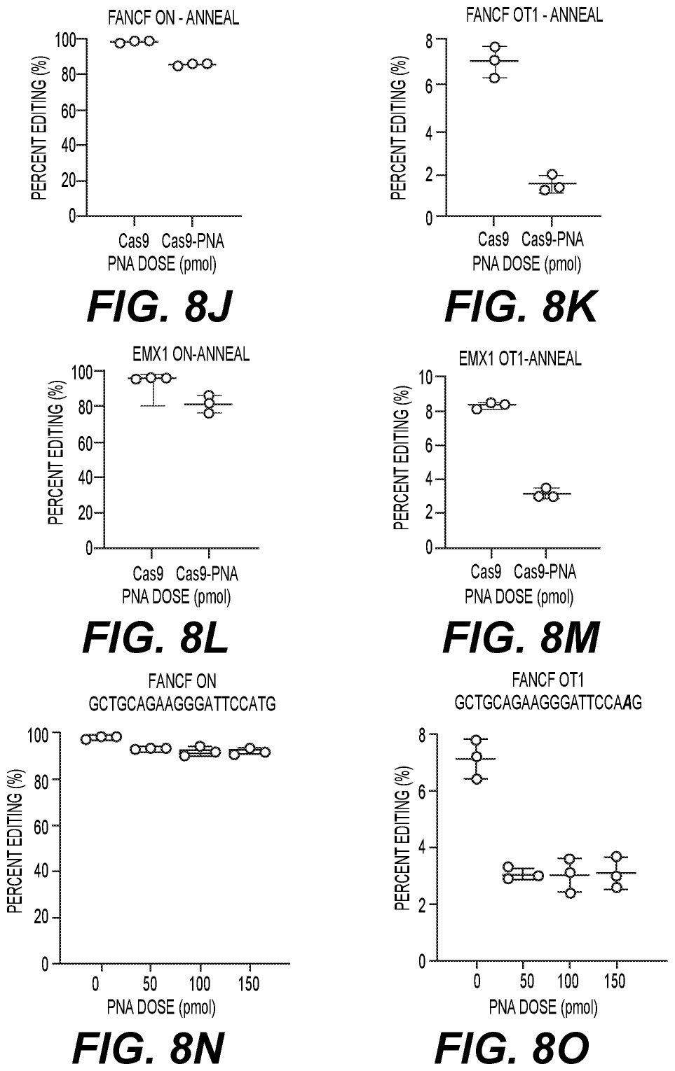

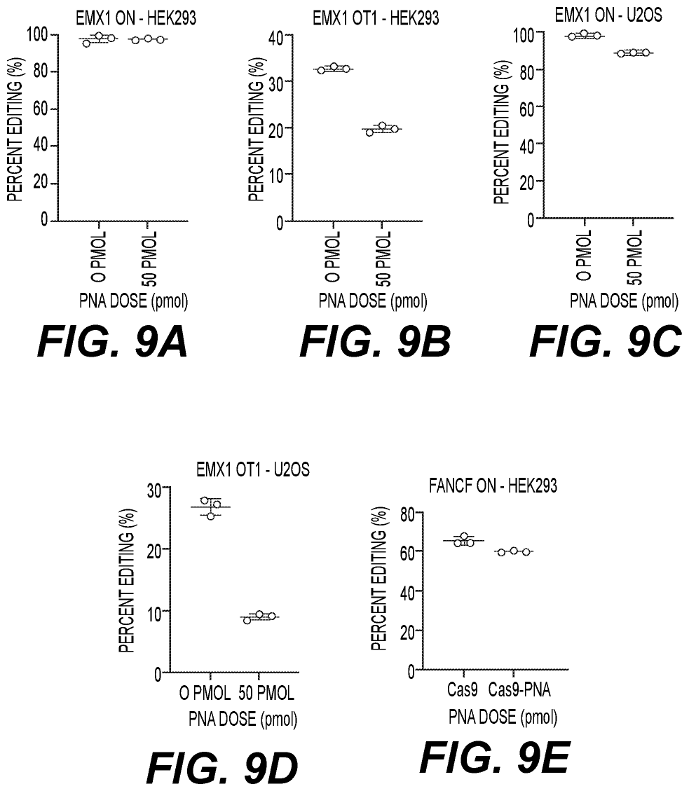

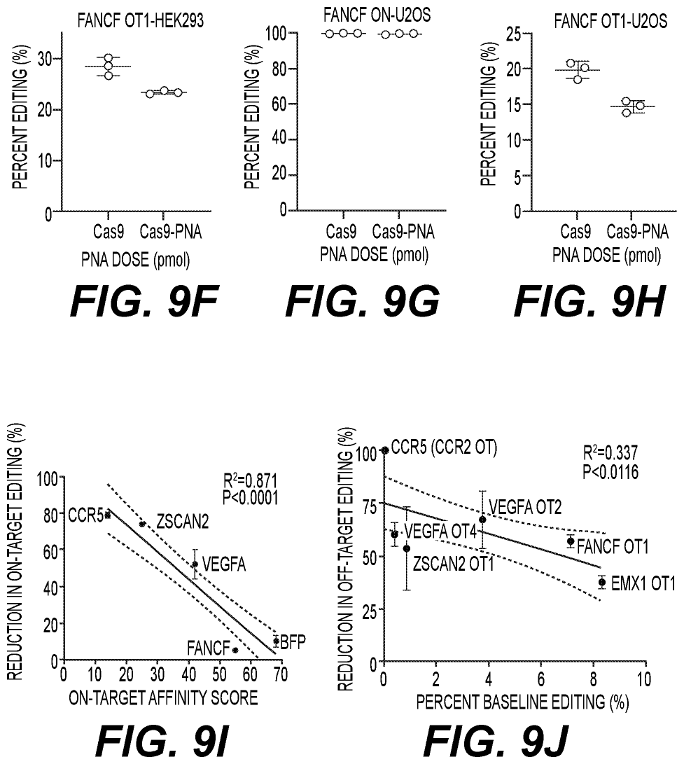

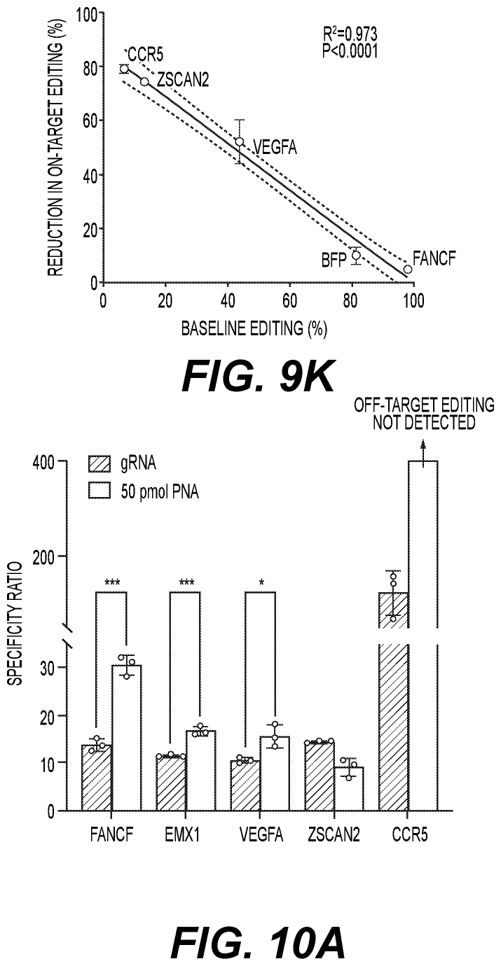

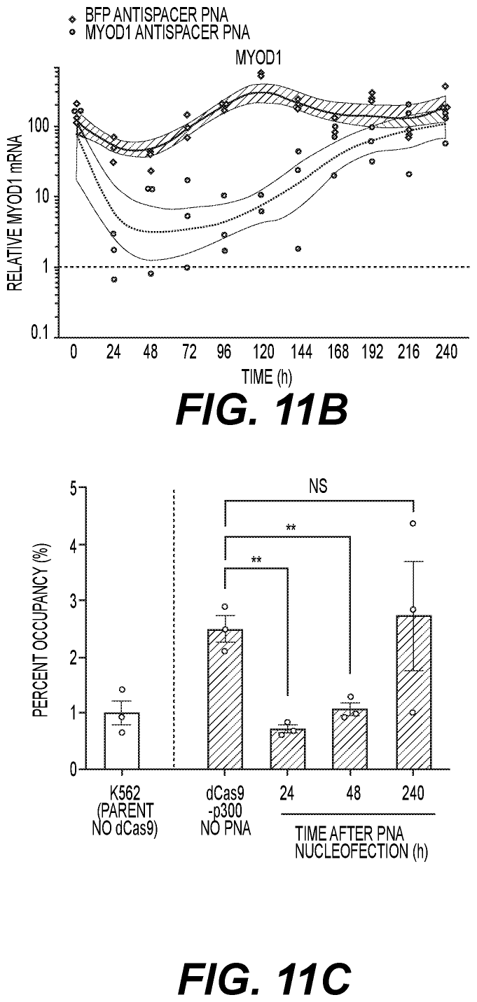

Graphing and statistical analysis were performed for each data set using Prism 9 (v9.3.0) software unless otherwise mentioned. For FIGS. 2C-2D, 3A-4D, 4A-4C, 8A-8I, and 8N-8O comparisons relative to control untreated conditions were calculated using Dunnett's multiple comparisons test after confirming significant two-way ANOVA interactions. For FIGS. 8J-8M, 9A-9K and 10A-10C, an unpaired student's t-test was used to compare untreated and PNA-treated conditions. For comparisons between conditions in FIGS. 2C, 10C, and 11C Tukey's multiple comparisons test was used after confirming significant two-way ANOVA interactions. For FIG. 9I-K a simple linear regression analysis was preformed and used to determine best-fit line and 95% CI. For FIG. 11A-B, Šfdák's multiple comparisons test was used to compare mean relative mRNA expression for each timepoint. FIGS. 11A-B, graphs and models were generated using JMP Pro (v15.0.0) software to fit a smoothing spline, k=0.5, and 95% CI.

Results Experiments were designed to determine if PNAs could inhibit Cas9 activity by binding directly to the sgRNA within the enzyme.

To investigate whether spacer targeted ssPNAs (antispacer PNAs) bind sgRNA sequences in vitro TAMRA fluorescent dye labeled BFP antispacer PNAs incubated with a BFP sgRNA and a control HBB sgRNA were used. Both PNAs were also incubated with gRNAs complexed to Cas9 to form Cas9 ribonucleoprotein (RNP).

These data demonstrate the sequence dependent binding of PNAs to the spacer element of unbound or Cas9-bound sgRNAs. TAMRA PNA signals only corresponded to sequence matched sgRNA sequences and showed a dose-dependent increase in binding signal for BFP Cas9 RNP. Additionally, a new faint gRNA bound PNA band appeared in PNA treated RNP lanes indicating PNAs have some ability to sequester sgRNA from the Cas9 enzyme. These data indicate that ssPNAs form PNA:RNA duplex structures with bound and unbound sgRNA spacer sequences. Results are illustrated in FIGS. 2A-2B.

Further, a non-targeting (GFP) PNA and a DNA oligo with the exact sequence as the “Anti” PNA proved entirely ineffective, indicating this effect is PNA specific and sequence specific. Results are illustrated in FIG. 2D.

### Example 2: Only the Sequence-Specific Targeting PNA Demonstrated Dose Dependent Cas9-Mediated Cleavage Inhibition

### Methods

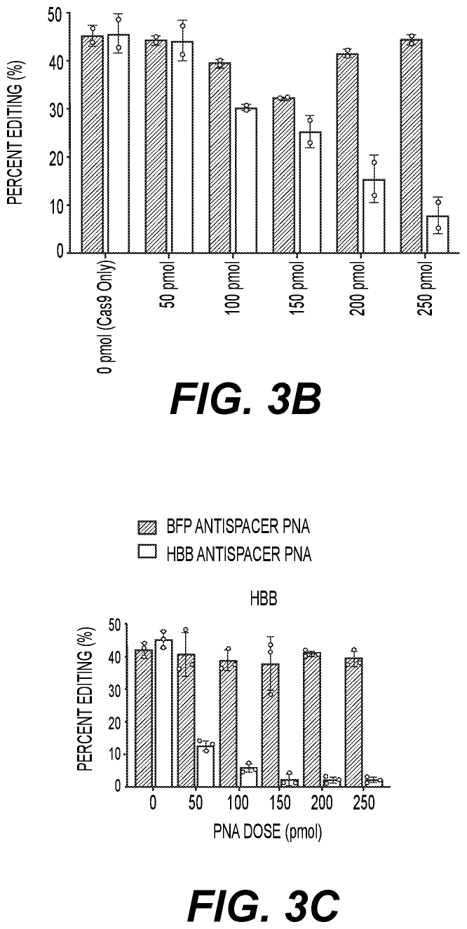

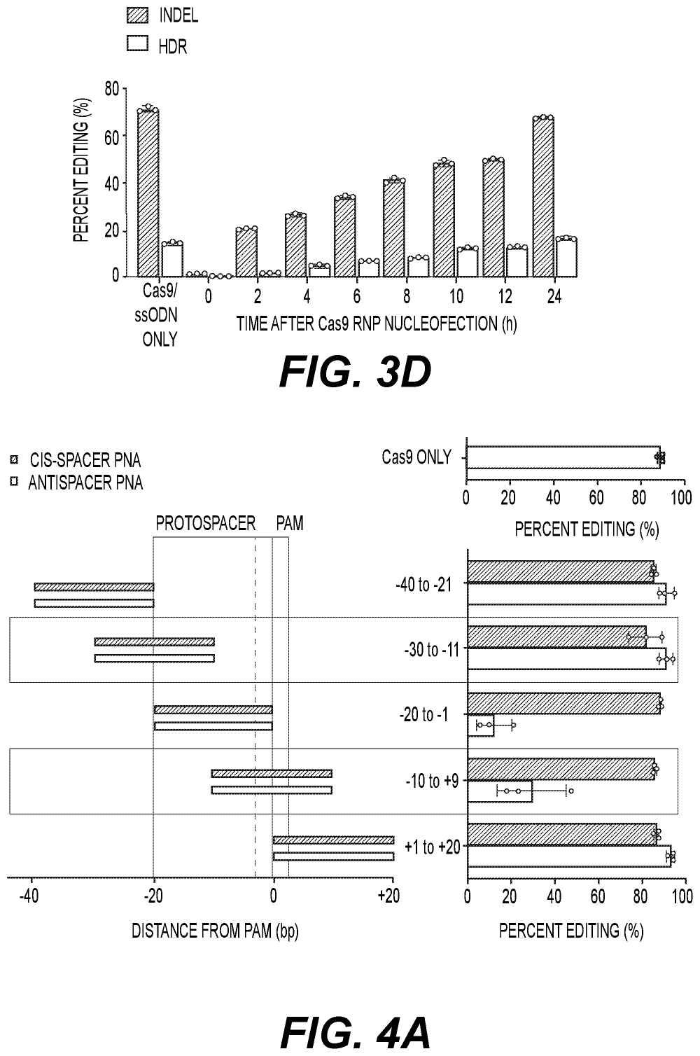

10⁶ stable BFP-expressing K562 cells were nucleofected (Lonza 4D Nucleofector) with a specified dose of BFP cis-protospacer PNA or HBB PNA (0-250 pmol). Two hours later, all conditions were nucleofected with 45 pmol of purified Cas9 RNP using a sgRNA targeted to the BFP gene or targeted to the HBB gene (R-01). 72 hours later, cells with BFP fluorescent drop out (via Cas9 indel formation) were quantified by flow cytometry (Beckman Coulter Cytoflex instrument) for the BFP target (FIG. 3A). For the HBB target (FIG. 3B-3C), 72 hours post-treatment gDNA was extracted (Promega ReliaPrep) and the region of interest was PCR amplified (NEB Phusion). Indel frequency was measured by T7 endonuclease assay (NEB—EnGen Mutation Detection Kit).

In time-course experiments in FIG. 3D, K562 samples were nucleofected with 5 μL RNP reactions with the addition of 100 pmol of ssODN donor in 1 μL of water. RNP nucleofected samples were incubated at 37° C. in complete media before being spun down and nucleofected with 250 pmol of PNA at specified time points. After PNA delivery cells were seeded in 2 mL of complete media and incubated for 96 hours prior to harvesting and analysis.

### Results

As a robust means to interrogate sequence specificity the Cas9 inhibitory effects of two PNAs were measured on two loci in the same cell line. For each locus (BFP and HBB), only the sequence-specific targeting PNA demonstrated dose dependent Cas9-mediated cleavage inhibition, while the non-targeting PNA had no such effect or relationship. See FIGS. 3A-3D.

These data demonstrate the excellent sequence specificity of PNAs with robust opposing controls and indicate multiplexing PNAs to multiple targets is a feasible approach. Experiments at the HBB locus (human hemoglobin subunit beta) also demonstrate a gene relevant to human disease can be targeted.

### Example 3: PNAs are Active Via Binding to sgRNA Spacer Sequence

### Methods

10⁶ stable BFP-expressing K562 cells were nucleofected (Lonza 4D Nucleofector) with specified dose (0-250 pmol, or 250 pmol if unspecified) of specified PNA with sequences homologous to (antispacer) or identical to (cis-spacer) the BFP gRNA spacer sequence. Two hours later, all conditions were nucleofected with 45 pmol of purified Cas9 RNP using a sgRNA targeted to the BFP gene. 72 hours later, cells with BFP fluorescent drop out (via Cas9 indel formation) were quantified by flow cytometry (Beckman Coulter—Cytoflex instrument).

### Results

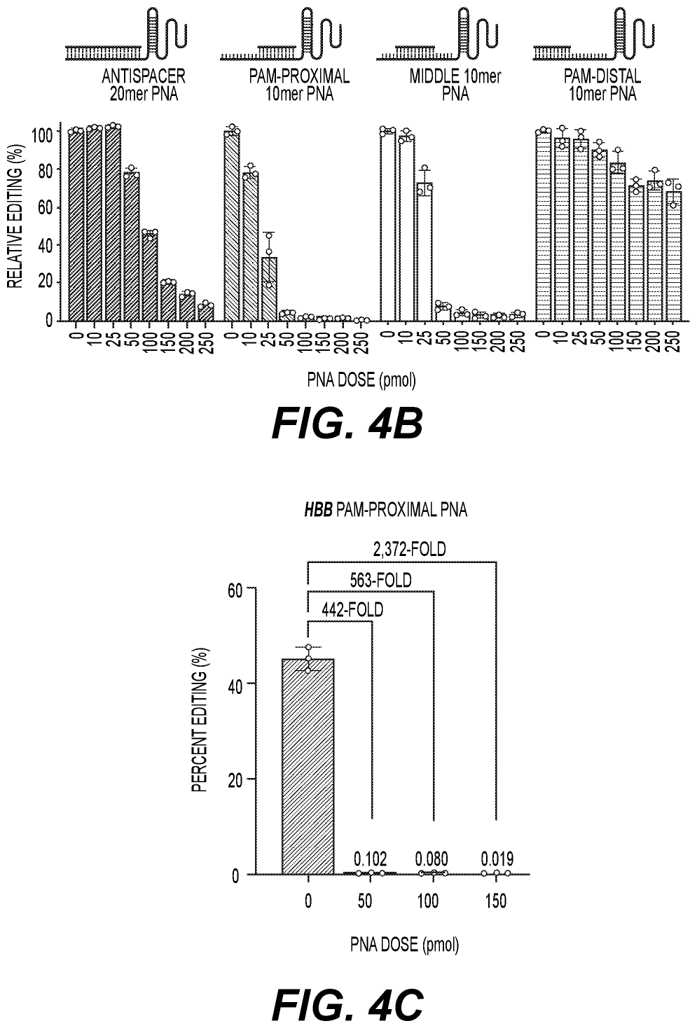

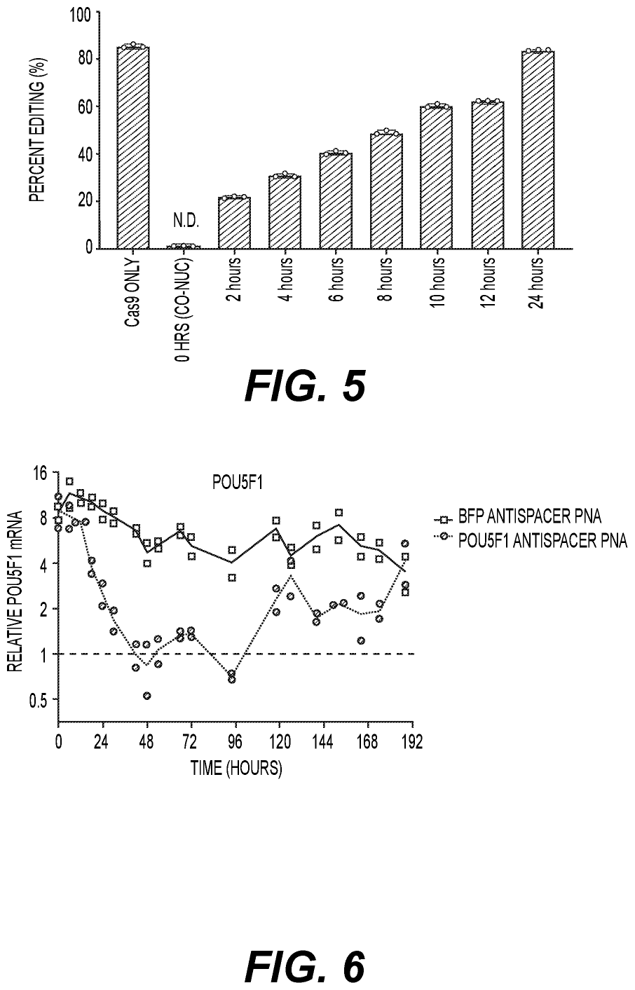

PNAs are known to bind genomic DNA sequences under certain circumstances. Thus, experiments were designed to rule out the possibility that PNAs may also modulate Cas9 activity by binding to genomic target sequences. To explore, 10 overlapping 20mer PNAs were designed to theoretically bind a region encompassing the protospacer, PAM, and flanking 20nt sequences on both strands of the BFP target DNA sequence. Following Cas9 treatment and analysis, an inhibitory effect for spacer sequence binding PNAs emerged with no effect outside this window. Only PNAs with complementarity to the PAM-proximal region of the spacer sequence demonstrated appreciable inhibitory activity. Notably, target strand oriented PNAs, PAM-distal antispacer PNAs, and PAM targeted PNAs were inactive. These data indicate that unmodified ssPNAs act primarily via sgRNA spacer sequence hybridization and do not have appreciable effects as duplex invasive genomic DNA binders. Results are illustrated in FIG. 4A.

In view of results indicating that the ssPNA act by specifically binding gRNA spacer sequences, either unbound, or in complex with Cas9, experiments were designed to determine which elements of the spacer were most important. Three short 10mer PNAs were designed to bind the PAM-proximal, middle, and PAM-distal spacer sequences of the gRNA. 10mer PNAs bound to the PAM proximal and middle spacer sequence of the gRNA demonstrated robust inhibitory activity, outperforming full 20mer antispacer PNAs. PAM-distal binding antispacer PNAs demonstrated an attenuated effect. These results confirm the importance of binding the PAM-proximal spacer sequence of the gRNA for inhibition. See FIG. 4B.

### Example 4: PNAs can be Delivered to Rapidly Inhibit Cleavage after Cas9 Delivery Across Timepoints

### Methods

10⁶ stable BFP-expressing K562 cells were nucleofected (Lonza 4D Nucleofector) with 45 pmol of purified Cas9 RNP using a sgRNA targeted to the BFP gene. At each specified time point 250 pmol of BFP Anti-protospacer PNA were introduced via nucleofection. For “Cas9 only” condition there was no second PNA nucleofection and for “0 hrs” 250 pmol of PNA was co-nucleofected with Cas9 in a single reaction. After each nucleofection samples were left in fresh media for the duration of the experiment to acquire edits. 72 hours after the “24 hrs” nucleofection, cells for all conditions with BFP fluorescent drop out (via Cas9 indel formation) were quantified by flow cytometry (Beckman Coulter—Cytoflex instrument).

### Results

While the Examples above demonstrate that introducing PNAs to cells prior to Cas9 can successfully block binding, experiments were also designed to test the ability of PNAs to block Cas9 after a window of activity. This approach is more closely aligned with a hypothetical use of Cas9 blocking PNAs in therapy and research applications in which Cas9 activity can be modified and titrated temporally. To investigate this, PNAs were nucleofected after introducing Cas9 across a 24-hour timespan. Interestingly, these results showed stepwise increases in Cas9 editing as a function of time.

These data demonstrate that PNAs can rapidly act to block Cas9 activity when delivered after Cas9 treatment. Each condition fails to accumulate additional edits after PNA introduction. The foregoing Examples collectively indicate that PNAs elicit their effects and persist on timescales longer than purified Cas9 RNP—likely due to their synthetic nature and resistance to endogenous nucleases or proteases.

### Example 5: PNAs can Modulate dCas9 Recombinant Systems

### Materials and Methods

TABLE 4
Spacer Sequences

| sgRNA | Spacer Sequence | PAM | Reference |
|---|---|---|---|
| POU5F1 | GGATGTTTGCCTAATGGTGG | TGG | Horizon Discovery |
| CRISPRa | (SEQ ID NO: 18) | | |
| sgRNA | | | |
| FANCF | GCTGCAGAAGGGATTCCATG | AGG | Kleinstiver, B. P. et al. |
| sgRNA2 | (SEQ ID NO: 19) | | High-fidelity CRISPR- |
| | | | Cas9 nucleases with no |
| | | | detectable genome-wide |
| | | | off-target effects. Nature |
| | | | (2016). |
| | | | doi: 10.1038/nature16526 |
| VEGFA | GGTGAGTGAGTGTGTGCGTG | | Fu, Y. et al. Nat. |
| sgRNA3 | (SEQ ID NO: 56) | | Biotechnol. 31, 822-826 |
| | | | (2013). |
| ZSCAN2 | GTGCGGCAAGAGCTTCAGCC | | Kleinstiver, B. P. et al.. |
| sgRNA1 | (SEQ ID NO: 57) | | Nature (2016). |
| | | | doi: 10.1038/nature16526 |
| CCR5 R30 | GTAGAGCGGAGGCAGGAGGC | | Cradick, T. J., Fine, E. |
| | (SEQ ID NO: 55) | | J., Antico, C. J. & Bao, |
| | | | G. Nucleic Acids Res. |
| | | | 41, 9584-9592 (2013). |
| EMX1 | GAGTCCGAGCAGAAGAAGAA | | Tsai et al., Nat. |
| sgRNA | (SEQ ID NO: 58) | | Biotechnol., 2014 |
| MYOD1 | AGGTTTGGAAAGGGCGTGC | CGG | Hilton, I.B. et al. |
| dCas9-p300 | (SEQ ID NO: 20) | | Epigenome editing by a |
| sgRNA | | | CRISPR-Cas9-based |
| | | | acetyltransferase |
| | | | activates genes from |
| | | | promoters and |
| | | | enhancers. Nature |
| | | | Biotechnology 33, 510- |
| | | | 517 (2015). |

CBE and FANCF guide RNAs were ordered as 100nt single guide RNAs (sgRNAs) from IDT with the specified protospacer sequence, POU5F1 sgRNA was ordered as purified lentiviral particles from Horizon Discovery.

TABLE 5
Target Sequences

| Targets | Target Sequence (100 bp segment) |
|---|---|
| POU5F1 | GGGTGGAGGAGAGGGAGGTGGGGGGAGAAACTGAGGCGAA |
| | GGATGTTTGCCTAATGGTGGTGGCAATGGTGTCTGTGGAAGG |
| | GGAAAACCGGGAGACACA (SEQ ID NO: 21) |
| FANCF | GTGCTTGAGACCGCCAGAAGCTCGGAAAAGCGATCCAGGT |
| (ON) | GCTGCAGAAGGGATTCCATGAGGTGCGCGAAGGCCCTACTT |
| | CCGCTTTCACCTTGGAGAC (SEQ ID NO: 22) |
| FANCF | TGACAGAGGTGCCTGGGTTTCATATGACTGACTTCGTATTCCC |
| (OT1) | CTTGGAATCCCTTCTGCAGCATAGTGGATGAGGTGAGACAGAG |
| | GCTGGTAACTCGGT (SEQ ID NO: 23) |
| MYOD1 | AGCCAGCGTTTCCCGCGGATACAGCAGTCGGGTGTTGGAG |
| | AGGTTTGGAAAGGGCGTGCCGGAGAGCCAAGTGCAGCCGC |
| | CTAGGGCTGCCGGTCGCTCC (SEQ ID NO: 24) |

Cas9 target sequences are underlined with protospacer in italics and PAM sequence in bold.

TABLE 6
PNA sequences

| PNA | Sequence |
|---|---|
| BFP Cis-Protospacer PNA | KKK-GCTGAAGCACTGCACGCCAT-KKK |
| (Non-Targeting BFP in expt 1) | (SEQ ID NO: 25) |
| POU5F1 CRISPRa PNA | KKK-CCACCATTAGGCAAACATCC-KKK |
| | (SEQ ID NO: 26) |
| FANCF OT1 PNA | KKK-GACTTCGTATTCCCCTTGGA-KKK |
| | (SEQ ID NO: 27) |
| FANCF PAM-Distal 10 mer PNA | K-CTTCTGCAGC-K (SEQ ID NO: 28) |
| MYOD1 p300 PNA | KKK-GCACGCCCTTTCCAAACCT-KKK (SEQ |
| | ID NO: 5) |

All PNAs were synthesized from unmodified PNA monomers and flanked with three lysine (K) residues on either side to improve solubility and promote strand invasion.

TABLE 7
TaqMan Assay Probes

| GENE | TaqMan Assay Probe ID |
|---|---|
| GAPDH | Hs02786624_g1 |
| POU5F1 | Hs00999632_g1 |
| MYOD1 | Hs00159528_m1 |

4×10⁶ stable dCas9-VRP/POU5F1 sgRNA expressing K562 cells (generated using lentivirus from Horizon Discovery), or K562 cells, were nucleofected with 250 pmol of specified PNA (non-targeting BFP or POU5F1) using a Lonza 4D nucleofector. At each specified time point 500 μL of cells were removed from each condition flask, spun down, and frozen. RNA was extracted from each pellet using QIAGEN Rneasy Mini Kits and cDNA was generated using Applied Biosystems High Capacity cDNA reverse transcription kit. rtPCR reactions were conducted for each sample using a StepOnePLUS Real-Time PCR System (Applied Biosystems) using Taqman Fast Advanced Master Mix and TaqMan gene specific probes for POU5F1 and GAPDH. Expression relative to the untreated parent K562 line was recorded as fold-change and calculated using the ΔΔC_{T} method.

### Results

Next, experiments were designed to investigate whether PNAs can modulate dCas9 recombinant systems in addition to Cas9 as a nuclease. A CRISPRa upregulated system was generated using a K562 stable line expressing dCas9-VPR (nuclease deficient Cas9 fused to transcriptional activators) and a guide RNA targeting the POU5F1 gene transcription start site. A PNA targeting this site was designed to see if it could prevent dCas9-VPR binding and temporarily drive down upregulated POU5F1 expression.

The results are presented in FIG. 6. These data demonstrate the ability of targeting PNAs to temporarily drive CRISPRa upregulated gene expression (triangles) down to the level of the parental K562 line (dashed line), before returning to the upregulated state. The same line treated with a non-targeting PNA (squares) remained upregulated throughout the experiment. Importantly, this supports the conclusion that PNAs impart their effect by preventing Cas9 binding to target DNA, rather than a nuclease-specific inhibitory effect. Furthermore, the full effect of PNAs in this context appears to last 2 to 3 days, and the effect appears to dissipate approximately 5 days after treatment.

### Example 6: ssPNAs can be Utilized to Reduce Off-Target Cas9 Events

### Materials and Methods

### Targeted Amplicon Sequencing

Genomic DNA was purified from 0.5×106 cells using Promega ReliaPrep gDNA Tissue Miniprep System (A2052) and eluted in 50 μL of water. Library prep used 100 ng of gDNA input with Ampliseq for Illumina Library Plus kit (20019101, Illumina) and used a custom primer pool designed to amplify a designed panel of genomic targets of interest (Table 9). Libraries were indexed, pooled, and loaded onto a Mid-output (300-cycles, Illumina, FC-420-1004) cartridge for paired-end sequencing on an Illumina Miniseq instrument. FASTQ reads were subjected to quality analysis (Basespace FASTQC) and analyzed for indel frequency using Cas-Analyzer assessment tool (parameters: Comparison range (R)—both ends of full amplicon sequence, Minimum frequency (n)=1). gRNAs used in this study are listed in Table 8, amplicon genomic coordinates listed in Table 9.

In the case of EMX1 ON and OFF target amplicons, both regions were amplified from 200 ng of purified gDNA by Phusion High-Fidelity Polymerase (NEB, M0530S) and pooled and purified using QIAGEN PCR purification kit (28104, Qiagen). Library prep used 200 ng of purified amplicon input with Illumina DNA Prep kit (20025519, Illumina). EMX1 libraries were similarly indexed, pooled, sequenced, and analysed as above.

10⁶ K562 cells were nucleofected (Lonza 4D Nucleofector) with specified dose (0-150 pmol) of FANCF PAM-distal 10mer PNA. Two hours later, all conditions were nucleofected with 45 pmol of purified Cas9 RNP using an sgRNA targeted to the FANCF gene. Cells were maintained in culture for 96 hours and then spun down and frozen. Genomic DNA was isolated using Promega ReliaPrep extraction kits. Amplicon libraries for high throughput sequencing were prepared using Illumina Ampliseq Library kits using a custom designed primer panel targeting regions of interest. Libraries were indexed, pooled, and paired end sequenced on an Illumina MiniSeq instrument. Sequencing data was analyzed for indel frequency using a validated Cas-Analyzer tool (Park, et al., Bioinformatics 33, 286-288 (2017)).

Precent reduction in on-target editing (calculated as: 100−(% editing(50 pmol)/mean % editing(0 pmol)*100) is graphed as a function of predicted on-target affinity score (Doench, Nature Biotechnology, 34, 184-191 (2016)) for FIG. 9I, and mean percent on-target editing (0 pmol) for FIG. 9K. A simple linear regression model was calculated for both relationships and R² and p-values are labeled on each graph. Colored line represents line of best fit and dotted lines represent 95% CI.

### Results

After noting an attenuated effect for on-target inhibition using PAM-distal targeted 10mer PNAs (in BFP model), experiments were designed to test whether these PNAs could be leveraged to improve on-target specificity. Previous work demonstrated that gRNA 5′ truncation or binding 5′ PAM-distal regions of sgRNAs with engineered RNA hairpins improved specificity in Cas9 systems, possibly by selectively inhibiting lower affinity off-target R-loop formation kinetics (Kocak, et al., “Increasing the specificity of CRISPR systems with engineered RNA secondary structures,” Nature Biotechnology 37, 657-666 (2019), Fu, et al., “Improving CRISPR-Cas nuclease specificity using truncated guide RNAs,” Nature Biotechnology 32, 279-284 (2014)). Thus, experiments were designed to test if modular application of distal binding 10mer PNAs (FIG. 7A) can be used to interrupt R-loop formation preferentially at off-target binding sites, thereby increasing specificity.

Indeed, introducing a PAM-distal targeted 10mer PNA against a FANCF sgRNA spacer sequence resulted in a 57.1% drop in editing at a known off-target site at the lowest dose of treatment (50 pmol, 1:1 PNA:sgRNA molar ratio), as compared to Cas9 RNP only (FIG. 8N). Meanwhile, in the same population, on-target editing only reduced by 4.9% (FIG. 8O). This 11.6-fold difference in editing reduction supports the hypothesis that gRNAs bound by PAM-distal PNAs incur an energetic penalty affecting successful R-loop formation (net AG increase). PAM-distal PNAs cause overall thermodynamic stability to decrease and are demonstrated to favor high-affinity on-target cleavage as opposed to lower energy off-target cleavage in FANCF.

As further evidence for the proposed mechanism of PAM distal 10mers, the affinity of gRNAs for on-target inhibition by 10mer PAM-distal PNAs was examined. Percent reduction in on-target editing strongly correlated with in silico predicted on-target affinity score³ (R²=0.871, P<0.0001) as well as measured baseline on-target editing frequency in K562 cells (R²=0.973, P<0.0001). Thus, these results indicate that the percent editing reduction predictably decreases as a function of both predicted and empiric measures of affinity. Because off-target sites, without exception, occur at lower affinity sites as compared to on-target editing it is believed that an improvement in specificity (on-target vs off-target frequency) will increase universally for Cas9 targets.

### Example 7: Pre-Annealing of PNA to sgRNA Spacer Sequence Reduces Off-Target Editing

### Methods

For pre-annealed PAM-distal PNA experiments, 50 pmol of sgRNA and 50 pmol of PAM-distal 10mer PNAs were incubated in a thermocycler at 37° C. for 30 minutes in a 5 μL reaction in 1×3.1 Buffer (B7203S, NEB) to allow annealing. PNA annealing to sgRNAs was confirmed by gel shift on 5% polyacrylamide gel. 45 pmol of SpCas9 (CP02, PNA BIO) was then added at room temperature and incubated for 10 minutes to allow RNP formation. Resulting reaction was delivered to 1×10⁶ K562 cells by a single nucleofection and treated as described above.

### Results

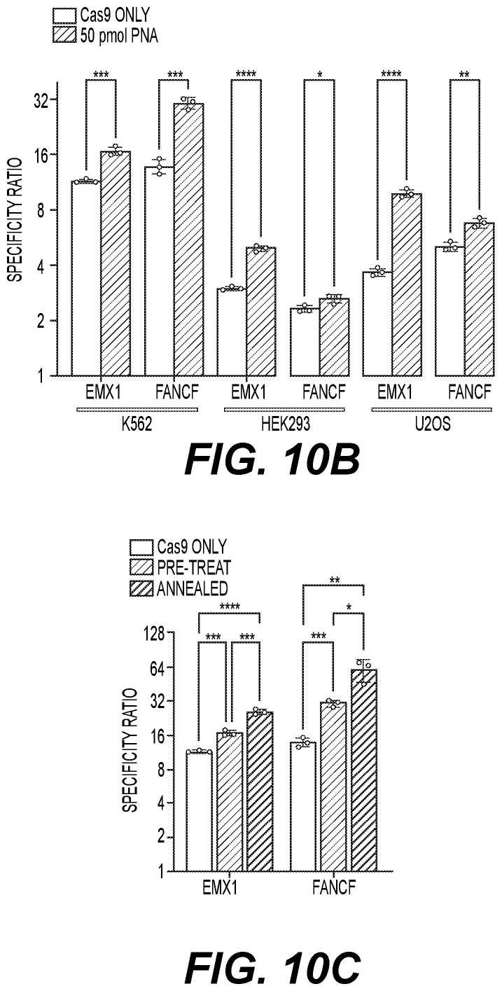

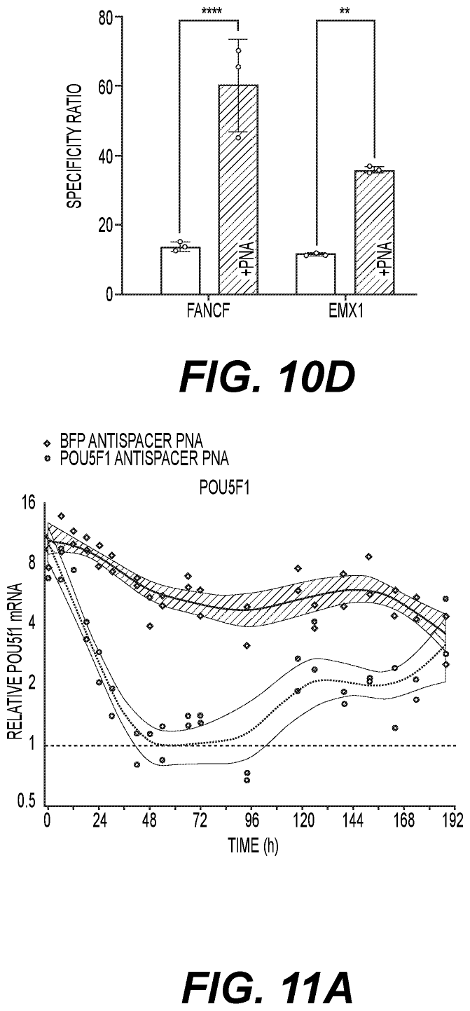

As set forth in FIGS. 8E and 8F, it was found that both pre-annealed FANCF and EMX1 Cas9 RNP-PNA complexes showed further improved specificity over pre-treatment approaches (FIGS. 9E-9H, 10C). This further improvement in specificity was due to more efficient PNA:RNA duplex formation in vitro. Importantly, this result indicates the feasibility of delivering PNA-bound ribonucleoprotein as a single functional complex.

### Example 8: Antispacer PNAs Modulate Acetyltransferase-Fused (dCas9-p300) Epigenetic Editing

### Materials and Methods

6×10⁶ stable dCas9-p300/MYOD1 sgRNA expressing K562 cells (Addgene #83889, Addgene #83925), were nucleofected with 250 pmol of specified PNA (non-targeting BFP or POU5F1) using a Lonza 4D nucleofector. At each specified time point 500 μL of cells were removed from each condition flask, spun down, and frozen. RNA was extracted from each pellet using QIAGEN Rneasy Mini Kits and cDNA was generated using Applied Biosystems High-Capacity cDNA reverse transcription kit. rtPCR reactions were conducted for each sample using a StepOnePLUS Real-Time PCR System (Applied Biosystems) using Taqman Fast Advanced Master Mix and TaqMan gene specific probes for MYOD1 and GAPDH. Expression relative to the untreated parent K562 line was recorded as fold-change and calculated using the ΔΔC_{T} method.

For FIGS. 11A and 11B, for each independent experiment, 6 replicates of 1×10⁶ K562 stable line cells and 1 μL of PNA diluted to 250 μM in water were nucleofected using Lonza SF cell line kit and 4D-Nucleofector. Cells were pooled into cultures and maintained in 10 mL of media at logarithmic growth phase (0.5-1.5×10⁶ cells/mL) throughout the experiment. For each time-point 0.5×10⁶ cells were removed from the culture for expression analysis (described below in RNA extraction and RT-PCR) and replaced with fresh complete media. gRNA sequences and sources used in this study are listed in Supplementary Table 4 and PNA sequences are listed in Supplementary Tables 3/3A. ssODN in Supplementary Table 8.

### RNA Extraction and RT-qPCR

For RNA extractions 0.5×10⁶ K562 cells were spun down, washed once with PBS, homogenized using QIAshredder columns (QIAGEN 79656) and processed using QIAGEN RNeasy mini kits (74106) before eluting in 50 μL of water. Resulting RNA was used as input for high-capacity cDNA Reverse Transcription Kit (Applied Biosystems) and resulting reaction was diluted 1:5 with water. RT-qPCR reactions were conducted in triplicate for each sample using a StepOnePLUS Real-Time PCR System (Applied Biosystems) and Taqman Fast Advanced Master Mix (Applied Biosystems) with TaqMan gene specific probes (Table 8). Expression relative to the untreated K562 parent line was recorded as fold-change and calculated from averaged CT values using the ΔΔCT method.

### ChIP-qPCR

K562 cells (6×10⁶ cells) were diluted to 0.4×10⁶ cells/mL and cross-linked with a final concentration of 1% formaldehyde (Sigma-Aldrich, 252549) for each IP. Chromatin was prepared and sheared according to manufacturer protocol using SimpleChIP Enzymatic Chromatin IP Kit (Cell Signaling, #9003) and QSONICA Q800R3 sonicator for nuclear lysis. For each condition, 10 μg of chromatin was incubated with 3 μg of H3K27ac antibody (abcam ab4729) rotating overnight at 4° C. Chromatin was incubated with 30 μL of protein G magnetic beads (Cell Signaling, #70024) and washed, eluted, reverse cross-linked, and purified according to manufacturer protocol. ChIP-qPCR reactions were conducted in technical triplicate for 2% input and IP samples using a StepOnePLUS Real-Time PCR System (Applied Biosystems), SimpleChIP Universal qPCR Mastermix (Cell Signaling, #88989), and MYOD1 target specific primers (Table 8). Percent occupancy values were calculated by percent input method from 2% input samples for each replicate.

### Cell Viability Assays

1×10⁶ K562 cells were nucleofected with specified doses of Cas9 RNP, PNA, or ssDNA using Lonza 4D-nucleofector. Cells were then serially diluted and seeded at 1,000 cells/well into 96-well plates and incubated for 72 hours. Finally, cell viability was measured in-plate using CellTiter-Glo Luminescent Cell Viability Assay (G7570, Promega) with a Synergy H1 Multi-Mode Microplate Reader (Biotek) and graphed as percent viability relative to mock (0 pmol) conditions for each experiment.

TABLE 8
gRNA list and sources

| Oligo | Sequence | Source | Citation |
|---|---|---|---|
| BFP | TCATGTGGTCGGGGTAGCGGCT | Keck Oligo Synthesis | Richardson et al., |
| SSODN | GAAGCACTGCACGCCGTACGTC | (in-house Yale | Nat. Biotechnol., |
| HDR | AGGGTGGTCACGAGGGTGGGCC | facility) | 2016. |
| Donor | AGGGCACGGGCAGCTTGCCGGT | | |
| | GGTGCAGATGAACTTCAGGGTC | | |
| | AGCTTGCCGTAGGTGGC (SEQ ID | | |
| | NO: 59) | | |
| MYOD1 | TCTCTCACGACCTGATTTCTACA | Keck Oligo Synthesis | |
| ChIP- | G (SEQ ID NO: 60) | (in-house Yale | |
| qPCR FWD | | facility) | |
| MYOD1 | CACCTCTGAGCTGACTTTCAATA | Keck Oligo Synthesis | |
| ChIP- | C (SEQ ID NO: 61) | (in-house Yale | |
| qPCR REV | | facility) | |
| Antispacer | ATGGCGTGCAGTGCTTCAGC | IDT (PAGE purified) | |
| SSDNA | (SEQ ID NO: 62) | | |
| (melting | | | |
| study) | | | |
| Antispacer | AUGGCGUGCAGUGCUUCAGC | IDT (HPLC purified) | |
| RNA | (SEQ ID NO: 63) | | |
| (melting | | | |
| study) | | | |
| Spacer | GCUGAAGCACUGCACGCCAU | IDT (HPLC purified) | |
| 20 mer | (SEQ ID NO: 64) | | |
| RNA | | | |
| (melting | | | |
| study) | | | |
| EMX1 ON | TCAGCTCAGCCTGAGTGTTG | Keck Oligo Synthesis | |
| FOR | (SEQ ID NO: 65) | (in-house Yale | |
| | | facility) | |
| EMX1 ON | TTGCCCACCCTAGTCATTGG | Keck Oligo Synthesis | |
| REV | (SEQ ID NO: 66) | (in-house Yale | |
| | | facility) | |
| EMX1 | CCCGCTTGTCCATGTCTAGG | Keck Oligo Synthesis | |
| OFF FOR | (SEQ ID NO: 67) | (in-house Yale | |
| | | facility) | |
| EMX1 | AGAAATGCCCAATCATTGATGC | Keck Oligo Synthesis | |
| OFF REV | T (SEQ ID NO: 68) | (in-house Yale | |
| | | facility) | |

TABLE 9
List of oligos, primers and probes

| Amplicon | Solution | | | Start | End |
|---|---|---|---|---|---|
| Id | Id | Region Name | Chromosome | Coordinate | Coordinate |
| AMPL1416190 | IAA23656_{—} | CCR5_R30_CCR2 | chr3 | 46357573 | 46357794 |
| | 181 | | | | |
| AMPL1416208 | IAA23656_{—} | CCR5_R30_ON | chr3 | 46372898 | 46373119 |
| | 181 | | | | |
| AMPL1416200 | IAA23656_{—} | VEGFA_sgRNA3_ON | chr6 | 43769547 | 43769772 |
| | 181 | | | | |
| AMPL1416202 | IAA23656_{—} | ZSCAN2_sgRNA1_OT2 | chr6 | 71610538 | 71610763 |
| | 181 | | | | |
| AMPL1028498 | IAA23656_{—} | HBB_R01_ON | chr11 | 5226839 | 5227061 |
| | 181 | | | | |
| AMPL1416210 | IAA23656_{—} | FANCF_sgRNA2_ON | chr11 | 22625758 | 22625976 |
| | 181 | | | | |
| AMPL1416191 | IAA23656_{—} | VEGFA_sgRNA3_OT2 | chr14 | 65102331 | 65102564 |
| | 181 | | | | |
| AMPL1416196 | IAA23656_{—} | ZSCAN2_sgRNA1_ON | chr15 | 84621625 | 84621848 |
| | 181 | | | | |
| AMPL1416206 | IAA23656_{—} | ZSCAN2_sgRNA1_OT1 | chr19 | 43913971 | 43914143 |
| | 181 | | | | |
| AMPL1416199 | IAA23656_{—} | FANCF_sgRNA2_OT1 | chr22 | 36556772 | 36556998 |
| | 181 | | | | |
| AMPL1416201 | IAA23656_{—} | VEGFA_sgRNA3_OT4 | chr22 | 37266755 | 37266980 |
| | 181 | | | | |
| EMX1 ON | Custom | EMX1_ON | chr2 | 72933701 | 72933963 |
| EMX1 OFF | Custom | EMX1_OFF | chr5 | 45358816 | 45359066 |

### Results

As additional evidence for modulating dCas9-based systems experiments were designed to test an acetyltransferase-fused system using a stable line with dCas9 p300 and a sgRNA targeted the MYOD1 gene promoter (Hilton, et al., Nature Biotechnology 33, 510-517 (2015)). The experiments were designed to test if PNAs can remove dCas9-p300 from the MYOD promoter to temporarily remove H3K27ac marks and drive down expression. Results MYOD expression was rapidly driven down within 24 hours with full-expression and reconstitution of the p300 system occurring around 168 hours (7 days). See FIGS. 11A-11C.

### Summary

The provided experiments establish that PNAs, with high-affinity sequence-specific binding to RNA, efficiently hybridize to gRNA spacer sequences to control the ability of Cas9 to interact with DNA sequences (FIG. 1A-1B). With an engineered hydrophobic protein-like backbone, PNAs can be especially amenable to stable nucleoprotein complexation and accommodation within the Cas9 binding channel.

PNAs were characterize as antisense modifiers of CRISPR-Cas9 with powerful inhibitory and modulatory properties. Antispacer PNAs were demonstrated to be a facile and design restriction-free platform and describe advancements in inhibitory potency and on-target specificity improvement while highlighting robust sequence selectivity. This study outlines an accessible tool to expand the versatility and improve the safety of Cas9 applications and describes an approach for the regulation of nucleoproteins via chimeric synthetic antisense nucleic acid molecules.

### In Vitro Characterization of Antispacer PNAs

For antispacer PNAs to impart an appreciable effect on Cas9 activity the affinity of a PNA for a given gRNA spacer sequence must exceed that of the DNA target strand. The thermal stabilities of a 20mer RNA oligonucleotide (including a spacer sequence designed to target the blue fluorescent protein (BFP) gene) bound to either a complementary target DNA sequence from the BFP gene (length 20nt), a complementary RNA oligomer (length 20nt), or a complementary “antispacer” PNA (length 20 bases) were compared. UV spectroscopy thermal analyses was applied to generate melting curves for DNA:RNA, RNA:RNA and PNA:RNA duplexes. PNAs demonstrated a far higher affinity for the RNA spacer sequence (Tm>100° C.) as compared to target sequence DNA or a complementary RNA oligonucleotide (Tm=56° C. and 69° C., respectively, FIG. 1B). Following this observation, an electrophoretic mobility shift assay (EMSA) was designed with purified Cas9 single guide RNA (sgRNA) and Cas9 ribonucleoprotein (RNP) with or without a TAMRA dye-labeled BFP antispacer PNA to determine the extent to which PNAs bind gRNA spacer sequences within RNP complexes in vitro. Results show that PNA-TAMRA signals strongly co-localized with both sequence-matched sgRNA and RNP bands (BFP target), but not sgRNA or RNP containing a heterologous spacer sequence (HBB target) (FIG. 2A-2B). These results indicate that PNAs stably bind complementary gRNA spacer sequences as part of the Cas9 RNP complex. Quantitative analysis of TAMRA-Cas9 RNP gel bands revealed a dose-dependent increase in PNA binding between 5 pmol (0.5×) and 10 pmol (1×) conditions (2.09-fold increase, p<0.0001, FIG. 2C). However, 20 pmol (2×) doses revealed no significant additional binding compared to 10 pmol (p>0.6), indicating that the Cas9 RNP may be efficiently bound at near 1:1 PNA:sgRNA ratios (FIG. 2C). Thus, in vitro, PNAs efficiently and tightly hybridize both free and Cas9-complexed gRNA spacer sequences at low stoichiometric ratios.

### Antispacer PNAs Achieve Sequence-Selective Cas9 Cleavage Inhibition in Human Cells

To determine if antispacer PNAs prevent Cas9 RNP-mediated cleavage and editing of a BFP reporter gene in human cells, PNAs were nucleofected into cells 2 hours prior to Cas9 RNP transfection, thus requiring PNAs to hybridize spacer targets in the intracellular environment. BFP-targeted 20mer antispacer PNA (designed to bind the gRNA targeting the BFP gene) was synthesized and compared to a nontargeting PNA (corresponding to a sequence in the GFP gene, length 20 bases) and to an antispacer single-stranded DNA oligonucleotide (ssDNA, length 20nt, with the same sequence as the BFP-targeted PNA) in Cas9 RNP-nucleofected K562-BFP cells. Flow cytometry measurements of BFP fluorescent drop out revealed a robust dose-dependent inhibitory effect of antispacer PNA on Cas9-mediated BFP gene disruption and indel formation at picomole doses (p<0.0001, FIG. 2D), whereas there was no effect of the antispacer DNA nor of the heterologous GFP PNA (p>0.3 across conditions).

To further establish the sequence-specific effect of PNAs on Cas9 activity, the inhibitory effects of two 20mer antispacer PNAs were compared: one designed to bind to the gRNA matching a target sequence in the BFP gene and the other to a gRNA targeting the human beta-globin gene (HBB). Both PNAs against Cas9 RNPs loaded with sgRNAs targeting the respective BFP or HBB sites were tested in the K562-BFP cell line. Editing was quantified either by flow cytometry for BFP or by high-throughput amplicon sequencing in the case of HBB. Only sequence-matched PNAs inhibited gene editing at their respective loci (p<0.0001 across conditions) with no detectable reduction in editing across doses for unmatched PNAs (p>0.48 across conditions, FIGS. 3A-3C), showing the specificity of action for antispacer PNAs in human cells.

Next, the possibility that PNAs might act, in part, by binding to target genomic DNA rather than, or in addition to, binding to the gRNA was considered. To investigate this possibility, 20mer PNAs complementary to both strands of the BFP target DNA sequence were tested, including the PAM and flanking 20nt regions and measured their effects on editing. Only PNAs with complementarity to the PAM-proximal gRNA spacer sequence showed any inhibitory activity (P<0.0001, FIG. 4A). PNAs with complementarity to DNA sequences but not the gRNA were ineffective (P>0.34 across conditions, FIG. 4A). PNAs inhibit Cas9 primarily via hybridization to gRNA spacer sequences rather than to the target DNA.

Whether PNAs could be introduced into cells at varying times after Cas9 RNP transfection to prevent further editing activity was next investigated. For these experiments, not only were BFP assayed for knockout by indel formation (BFP−, GFP−) but also for homology directed repair (HDR)/template-mediated editing of BFP to GFP when Cas9 RNP is combined with a single-stranded oligodeoxynucleotide (ssODN) donor instructing a single codon change (BFP−, GFP+). It was determined to assay for both HDR and NHEJ repair outcomes, to confirm the scaled effects of Cas9 inhibition for both repair pathway outcomes. ssODN templates were designed to avoid complementarity with the PNA to prevent hybridization events. The antispacer PNA demonstrated the ability to rapidly and proportionally block both indel formation (NHEJ) and template-mediated editing (HDR) across time-points up to 24 hours, at which time most gene modification had occurred, and the overall editing levels were close to those seen in non-PNA treated cells (Cas9 RNP/ssODN only, FIG. 3D). Together, these studies demonstrate that antispacer PNAs act in human cells in a sequence-specific fashion to robustly prevent on-target Cas9-mediated cleavage and editing. These results show that this effect can be dose and time-adjusted to tune overall levels of activity.

### Short PAM-Proximal Antispacer PNAs Demonstrate Superiorly Potent Cas9 Cleavage Inhibition

Next, to evaluate the impact of PNA length and binding position on Cas9 inhibition, smaller 10mer antispacer PNAs were synthesized to scan across the BFP spacer sequence and determined their effects on editing. In addition to the fully complementary 20mer antispacer PNA, 10mer PNAs were synthesized to bind across the PAM-proximal, middle, and PAM-distal spacer sequences of a BFP-targeted gRNA (FIG. 4B). A 10mer PNA targeted to the PAM-proximal spacer demonstrated highly effective Cas9 inhibitory activity in cells, with a 24.5-fold reduction in editing noted at 1:1 PNA:sgRNA molar ratios (50 pmol) and a 342.6-fold reduction at 5:1 ratios (250 pmol, FIG. 4B). A separate amplicon sequencing analysis of a 10mer PAM-proximal PNA targeted to an HBB gRNA in K562 cells also demonstrated excellent inhibition (FIG. 2D). For HBB target editing a 443-fold reduction at 1:1 PNA:sgRNA ratios (50 pmol, 45.2% vs 0.102% editing) was measured and near complete abrogation of editing by 150 pmol doses (6 total indels detected across 28,104 analyzed amplicons over three replicates, mean 2,372-fold reduction, FIG. 4C) was observed. Middle antispacer PNAs also demonstrated potent inhibition (32.4-fold reduction at 250 pmol), though slightly weaker than PAM-proximal targeting (FIG. 4C). Improved inhibitory activity of 10mer PNAs over the full 20mer antispacer PNA may be attributed to size and the ability of the molecule to enter the Cas9 binding channel. The PAM-distal antispacer PNA, however, demonstrated an attenuated ability to block BFP editing across doses, achieving only 10% reduction at 50 pmol doses (versus 96.0% reduction for PAM-proximal PNA) and 32% reduction at 250 pmol doses (versus 99.7% for PAM-proximal). These data illustrate the importance of the PAM-proximal spacer sequence for Cas9 targeting and inhibitory activity and are consistent with previous studies on the mechanisms and kinetics of Cas9 R-loop formation (Farasat and Salis, PLoS Comput Biol, 12, e1004724-e1004724 (2016)). Importantly, at very low stoichiometric doses (1:1), PAM-proximal PNAs demonstrated superior inhibition activity in cells (at least 10-fold improvement) as compared to published values for purified inhibitors such as Acrs, small molecules, DNA-based aptamers, and small modified nucleic acid inhibitors, indicating the high potency of appropriately designed PNAs for Cas9 inhibition (Maji, et al. Cell, 177, 1067-1079 e1019 (2019)); Barkau, et al., Nucleic Acid Ther, 29, 136-147 (2019); Zhao, et al., Nucleic Acids Research, 49, 1330-1344 (2020)).

Short PAM-Distal Antispacer PNAs Modulate gRNA Target Affinity to Improve On-Target Specificity

Whether antispacer PNAs could be leveraged to improve the on-target specificity of Cas9 RNP activity, defined here as the ratio of on-target to off-target editing rates, was assessed. Previous work demonstrated that manipulating 5′ PAM-distal regions of sgRNAs by truncation or with engineered RNA hairpins improves specificity in Cas9 systems, likely by selectively preventing lower affinity off-target R-loop formation (Kocak, et al. Nature Biotechnology, 37, 657-666 (2019); Fu, et al. Nature Biotechnology, 32, 279-284 (2014)). Further, strategic gRNA spacer sequence base modifications have also been used to affect thermodynamic barriers and favorably modulate specificity (Ryan,. et al. Nucleic Acids Research, 46, 792-803 (2017)).

PAM-distal PNA:gRNA duplexes with 10mer PNAs in cells may similarly interrupt R-loop formation preferentially at off-target binding sites, thereby increasing specificity (FIGS. 7A-7C). To test this, PAM-distal antispacer 10mer PNAs were designed for gRNAs targeting five selected genomic sites in the FANCF, EMX1, ZSCAN2, CCR5, and VEGFA genes, each with known off-target activity in the genome. K562 cells were pre-treated with nucleofected PAM-distal 10mer PNAs followed by Cas9 RNP 2 hours later and quantified on-target and respective off-target editing rates using high-throughput amplicon sequencing. In the case of the clinically relevant target FANCF, a 10mer PAM-distal PNA reduced overall off-target editing by 57.1% at a 1:1 PNA:sgRNA ratio (50 pmol, 7.13% vs 3.07%) while on-target editing was reduced by only 4.9% (50 pmol, 98.0% vs 93.2%, FIGS. 8N-80).

This 11.6-fold difference in percent editing reduction supports the hypothesis that gRNAs bound by PAM-distal PNAs incur an energetic penalty affecting successful R-loop formation (net AG increase, FIG. 7C). PAM-distal PNAs cause overall thermodynamic stability to decrease and are demonstrated to favor high-affinity on-target cleavage as opposed to lower energy off-target cleavage. As further evidence, it was found that lower affinity gRNAs were more susceptible to on-target inhibition by 10mer PAM-distal PNAs. Percent reduction in on-target editing strongly correlated with in silico predicted on-target affinity scores (Doench, et al. Nature Biotechnology, 34, 184-191 (2016)) (R2=0.853, P<0.0001, FIG. 9I) as well as measured mean baseline on-target editing in K562 cells (R2=0.976, P<0.0001, FIG. 9K). Thus, it was demonstrated that percent editing reduction for PAM-distal PNA-bound gRNAs predictably decreases as a function of both predicted and empiric measures of affinity.

In agreement with these models, off-target editing decreased by a larger margin as compared to on-target editing for four out of five loci, and sites with the largest differences in percent on-target versus off-target editing demonstrated the largest increases in specificity (FIG. 10A, Supplementary FIG. 8A-8I, 9J). In the case of ZSCAN2, both on-target and off-target editing decreased by a large margin (80.3% and 66.3%, respectively), and an overall increase in specificity was not observed (FIGS. 8D-8E; FIG. 10A). For CCR5, on-target editing decreased by 79.1% while off-target editing in the CCR2 gene was rendered undetectable (FIGS. 8F-8G; FIG. 10A). Based on these relationships, it is believed that further fine-tuning of PNA length and binding strength may overcome high on-target reduction in lower affinity targets, such as ZSCAN2 and CCR5, while maintaining large reductions in off-target editing.

Next, the ability of PAM-distal PNAs to improve on-target specificity in additional cell lines was explored. gRNAs with the highest off-target activity from the analysis were selected, FANCF and EMX1, and pre-treated K562, HEK293, and U2OS cell lines with PAM-distal PNAs prior to Cas9 RNP nucleofection. As expected, significantly increased specificity ratios were observed for both targets across all three cell lines (P<0.05 across conditions, (FIG. 10B). Notably, K562, HEK293, and U2OS lines demonstrated specificity improvements across widely variable baseline off-target editing activity for both EMX1 (8.33%, 32.7% and 26.8%, respectively, FIGS. 8H-8I, FIGS. 9A-9D) and FANCF (7.13%, 28.4%, and 19.8%, respectively, FIGS. 8N, 8O and FIGS. 9E-9H).

Finally, pre-annealing a PAM-distal 10mer PNA to FANCF and EMX1 sgRNAs prior to Cas9 RNP formation for delivery as a single complex was tested. Both pre-annealed FANCF and EMX1 Cas9 RNP-PNA complexes showed further improved specificity over pre-treatment approaches (FIG. 10C, and FIGS. 9J-8H). This further improvement in specificity is due to more efficient PNA:RNA duplex formation in vitro. Importantly, this result indicates the feasibility of delivering PNA-bound ribonucleoprotein as a single functional complex.

Together, these data demonstrate that antispacer PNAs can be rationally designed to impart two distinct modulation effects on Cas9 activity: superior sequence-specific inhibition via PAM-proximal spacer targeting, and affinity manipulation and specificity enhancement via PAM-distal binding. Antispacer PNAs modulate spatiotemporal activity of dCas9 fusion systems in a sequence-specific manner.

Beyond use as a nuclease for gene knockout and editing, nuclease-deficient variants of Cas9 (dCas9) are widely used to create fusion systems for sequence-specific localization of effectors. Precise control over these systems via sequence-specific dCas9 binding modulation further expands the versatility of applications and provide improved control for biological investigations and applications.

Given the robust effects of antispacer PNAs on Cas9-mediated cleavage, the extent to which PNAs can modulate the functional activity of two well-established dCas9 fusion effectors was tested: a CRISPRa (dCas9-VPR) transcriptional activator and a histone acetyltransferase-fused epigenome editor (dCas9-p300). PNAs were applied to a stably integrated CRISPRa system in K562 cells targeting a POU5F1 promoter to constitutively upregulate the gene, and measured gene expression over time following PNA transfection (FIG. 4A). After the introduction of 250 pmol of a 20mer POU5F1-targeted antispacer PNA, POU5F1 expression rapidly dropped to the level of the parent line expression by 48 hours (FIG. 11A). By 120 hours (day 5) expression began to return to its upregulated baseline (FIG. 11A). This effect was specific to the sequence-matched antispacer PNA, as a nontargeting (BFP) PNA control did not suppress CRISPRa activity at the POU5F1 locus, which remained upregulated and highly expressed (FIG. 11A). Next, PNAs were used to modulate the appearance and disappearance of epigenetic marks using a fused acetyltransferase (dCas9-p300) epigenetic editing system (Hilton, et al., Nature Biotechnology, 33, 510-517 (2015)). At baseline this system produced increased H3K27ac histone modification near the promoter region of the MYOD1 gene and demonstrated robust gene upregulation (FIGS. 11B-11C). MYOD1 gRNA-targeted PNAs were able to drive down epigenetic-mediated gene upregulation by 24 hours (FIG. 11B-11C). By 168 hours (7 days) expression returned to the fully upregulated state (FIG. 11B). Corroborating these observations, a significant decrease in H3K27ac occupancy at the MYOD1 promoter region was observed by 24 and 48 hours after PNA treatment followed by a subsequent increase at 240 hours as measured by ChIP-qPCR (FIG. 11C). Collectively, these data demonstrate that antispacer PNAs can temporally manipulate stable dCas9 fusion systems and influence downstream biology. Results from two independent systems indicate that PNA blocking happens rapidly and in a sequence-specific manner following a single PNA dose, with full effects noted by 24-48 hours and persistence for at least an additional 2-3 days after that.

In sum, a class of synthetic high-affinity gRNA-binding PNA molecules were designed, synthesized, and characterized as being capable of rationally manipulating Cas9 interactions with dsDNA targets. Using PNAs with PAM-proximal homology,potent Cas9 inhibition in human cells was shown by targeting consequential interactions driving R-loop formation. This approach results in superior inhibitory potency at low stoichiometric ratios with the important added benefit of sequence specificity. In contrast to other approaches to Cas9 inhibition, PNAs may be applicable as sequence-selective regulators of multiplexed Cas9 systems such as large-scale screens.

This work also characterized the affinity-dependent effects of PAM-distal targeted antispacer PNAs and demonstrate that this approach can be leveraged to predictably improve Cas9 specificity. Because off-target sites, by definition, occur at lower affinity sites as compared to on-target loci, it is believed that specificity will increase for most Cas9 targets. However, lower affinity gRNAs were less amenable to specificity improvement using PAM-distal 10mer PNAs. Importantly, PAM-distal PNAs can be further adjusted in length and chemically modified via sidechain substitution at the gamma(γ) position in the polyamide backbone to fine-tune binding strength for altered effect (Dragulescu-Andrasi, et al. Journal of the American Chemical Society, 128, 10258-10267 (2006); Verona, et al., Scientific reports, 7, 42799-42799 (2017)). For example, for low-affinity targets such as CCR5 and ZSCAN2, shorter 8mer or 6mer PAM-distal PNAs may result in less on-target inhibition effects and more optimally adjust specificity. Conversely, for high-affinity targets such as FANCF or EMX1, increasing PNA affinity can further improve specificity by further reducing off-target effects and maintaining high on-target editing. In addition to increasing PNA length beyond 10 bases, gamma(γ) position modification with chemical sidechains such as a hydroxymethly group (serine) or polyethlene glycol (mini-PEG) can improve PNA helical organization and increase binding strength (Bahal, et al. Nature Communications, 7, 13304 (2016)). Notably, previous work (Verona, et al., Scientific reports, 7, 42799-42799 (2017)) has shown that one can computationally predict the effects of length and γ-modifications on PNA helical preorganization to fine-tune PNA affinity for complementary RNA targets. Coupled with models predicting antispacer PNA influence on gRNA target affinity, as provided herein (FIGS. 9I-9K), these tools have the potential to predict optimized PNA formulations for individual gRNA targets. Incorporating predicted gRNA affinity, off-target rates, and PNA binding strength, future in silico methods may identify targets most suitable for off-target mitigation using PNAs. This phenomena may further improve Cas9 specificity across targets to reduce genotoxicity and improve safety for use in human therapeutics.

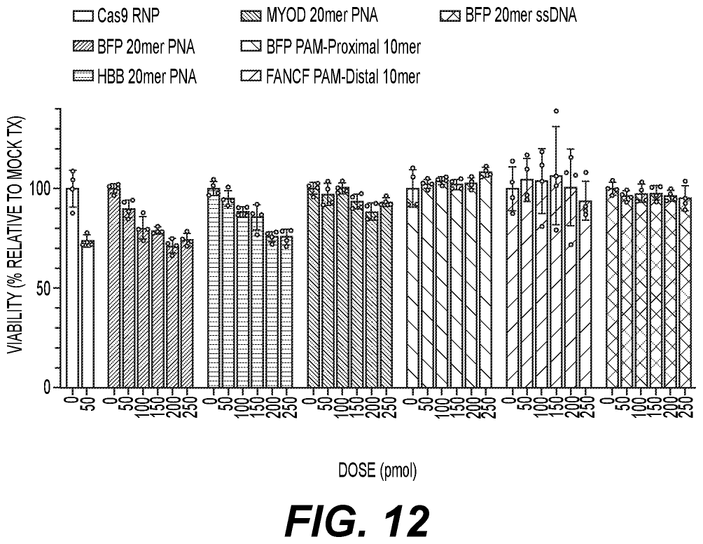

While PNAs offer clear advantages as a synthetic nuclease-resistant technology with powerful nucleic acid binding properties, their application poses some challenges (Pellestor and Paulasova, European Journal of Human Genetics, 12, 694-700 (2004); Gupta, et al. J Biotechnol, 259, 148-159 (2017)). Firstly, PNAs are not as commercially accessible as conventional nucleic acids, and historically PNA synthesis required laborious manual chemical synthesis and relevant expertise. However, with the availability of suitable monomers, methods as outlined herein are available to automate PNA synthesis using an in-lab benchtop peptide synthesizer and HPLC system (see Materials and Methods for both automated and manual synthesis protocols (Braasch, et al.(2002) Curr Protoc Nucleic Acid Chem, Chapter 4, Unit 4.11.). Using this approach, PNAs were designed, synthesized, and purified ready for delivery within 72 hours and with minimal hands-on time. Additionally, with a hydrophobic polyamide backbone, PNAs may demonstrate poor aqueous solubility and aggregation relative to classic nucleic acids, especially sequences with high purine content. Notably, this effect is minimized for shorter PNAs (<30 bases), such as those used in this study, and can be further offset with the inclusion of hydrophilic N- and C-terminal L-lysine residues. Toxicity, potentially related to sequence, may also present possible challenges for application. Viability studies in nucleofected human K562 cells using a variety of PNA sequences and lengths used in this study demonstrated minimal cellular toxicity across doses (FIG. 12). Prior studies have similarly noted low toxicity across a range of PNA doses in other cell types including U251 and HeLa lines and in primary human CD34⁺ cells, (Turner, et al. Nucleic Acids Research, 33, 6837-6849 (2005); Gasparello, et al., Scientific Reports, 9, 3036 (2019); McNeer, et al., Mol Ther, 19, 172-180 (2011)). These findings are consistent with previous studies in vivo in mice that found little to no toxicity across multiple delivery methods for systemic treatments of PNA formulations (Chaubey, et al. Oligonucleotides, 18, 9-20 (2008); McNeer, et al. Gene Therapy, 20, 658-669 (2013); and Schleifman, et al., Molecular Therapy—Nucleic Acids, 2, e135 (2013)).

In contrast to sequence-restricted or protein-coded approaches, an accessible and rapidly implementable platform for CRISPR-Cas9 modulation that features facile PNA oligomer design and application has been established. This technology provides new methods for enhanced control over Cas9-derived experimental systems and may offer a tool to improve the safety of human therapeutic applications. Further, because antispacer PNAs target gRNA sequences, they are theoretically applicable to all current and future Cas9 and dCas9-based systems, and likely other RNA-guided RNP systems. Beyond CRISPR-Cas9, the principles applied in this study can inform future approaches to manipulate the character and activity of nucleoproteins via stable antisense binding and modification with PNAs.

Economos, et al., “Antispacer peptide nucleic acids for sequence-specific CRISPR-Cas9 modulation,” Nucleic Acids Research, gkac095, (2022), doi.org/10.1093/nar/gkac095, pages 1-12 and all supplemental materials and tables associated therewith are specifically incorporated by reference herein in their entireties.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

We claim:

1. A composition for spatiotemporal control of CRISPR Cas, comprising
(a) a single stranded anti-spacer peptide nucleic acid oligomer (ssPNA),
wherein the ssPNA comprises a nucleobase sequence that hybridizes to part of, or all of the spacer sequence of a spacer-sequence containing CRISPR RNA that binds to a protospacer sequence of a CRISPR/Cas target site in a double stranded DNA.

2. The composition of claim 1, further comprising
(b) the spacer sequence-containing CRISPR RNA,
wherein at least 50%, up to 100% of the nucleotides within the spacer sequence are complimentary to the nucleobases in the ssPNA.

3. The composition of claim 2,
wherein the at least 50% up to 100% of the PNA nucleobases are hybridized to the complimentary nucleotides in the spacer sequence.

4. The composition of any one of claims 1-3, wherein the ssPNA hybridizes to the CRISPR RNA by Watson-Crick binding only.

5. The composition of any one of claims 1-4,
wherein the ssPNA comprises from 5 to 25 contiguous nucleobases, inclusive, or any subrange of specific integers there between,

6. The composition of any one of claims 1-5,
wherein the spacer sequence of the spacer-sequence containing CRISPR RNA comprises from 15 to 25 contiguous nucleotides, inclusive, or any subrange of specific integers there between.

7. The composition of any one of claims 1-6, wherein
(i) the nucleobase sequence of the ssPNA hybridizes to any integer number between 1-25, 5-25, 10-25, 15-25, 1-20, 5-20, 5-15, or 8-12 nucleotides of the spacer sequence of the CRISPR RNA; and/or
(ii) the nucleobase sequence of the ssPNA hybridizes to between about 25% and about 100%, inclusive, of the nucleotides of the spacer sequence of the CRISPR RNA.

8. The composition of any one of claims 1-7, wherein the hybridization comprises 0, 1, 2, 3, 4, 5 or more mismatches, gaps, and/or insertions

9. The composition of any one of claims 1-8, wherein the ssPNA does not form a PNA:DNA:DNA triplex, or a PNA:DNA:PNA triplex with the CRISPR RNA or at the CRISPR/Cas target site.

10. The composition of any one of claims 1-9, wherein the nucleobase sequence comprises
(i) part of the protospacer sequence; or
(ii) the entire protospacer sequence.

11. The composition of any one of claims 1-10, wherein, the CRISPR RNA is or comprises
(i) a crRNA; or
(ii) a tracrRNA; or
(iii) a guide RNA (gRNA); or
(iv) a single guide RNA (sgRNA).

12. The composition of any one of claims 1-11, wherein the ssPNA comprises between about 5 and about 50 PNA residues in length, more preferably about 8 to about 30 residues, or about 10 to about 20 residues, or about 8 to about 12 residues, or about 10 residues in length.

13. The composition of any one of claims 1-12, wherein the CRISPR RNA that targets the CRISPR/Cas target site exhibits reduced binding, and optionally cannot bind to the protospacer sequence, when the ssPNA is present.

14. The composition of any one of claims 1-12, wherein a Cas enzyme that binds to the CRISPR RNA that targets the CRISPR/Cas target site
(i) exhibits reduced binding to the CRISPR/Cas target site; or
(ii) cannot bind to the CRISPR/Cas target site,
when the ssPNA is present.

15. The composition of any one of claims 1-14, wherein the ssPNA comprises at least an integer number between 5 and 40, inclusive, of bases between −20 and −1 of the target site's protospacer sequence,
wherein −1 is the last nucleobase of the protospacer and +1 is the first nucleobase of the protospacer adjacent motif (PAM) of the target site.

16. The composition of any one of claims 1-15, wherein some of, or all of the PNA residues are modified at the gamma position,
optionally wherein the modification is diethylene glycol, or substitution with an amino acid side chain,
optionally wherein the amino acid side chain is selected from alanine, serine, threonine, cysteine, valine, leucine, isoleucine, methionine, proline, phenylalanine, tyrosine, aspartic acid, glutamic acid, asparagine, glutamine, histidine, lysine, and arginine, or the derivatives thereof.

17. The composition of any one of claims 1-16, wherein the ssPNA comprises heterocyclic bases selected from uracil, thymine, cytosine, adenine, guanine, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives.

18. The composition of any one of claims 1-17, wherein the ssPNA comprises one or more positively charged moieties,
optionally wherein
(i) the one or more positively charged moieties are selected from lysine and arginine; and/or
(ii) the one or more positively charged moieties are present at the N-terminus, C-terminus.

19. The composition of any one of claims 1-18, wherein the ssPNA comprises at least 20 PNA residues, and comprises one, two or three lysines at the N-terminus, or the C-terminus, or both the N-terminus and C-terminus.

20. The composition of any one of claims 1-19, wherein the ssPNA comprises a nucleobase sequence of any one of the sequences set forth in Table 3 or Table 3A.

21. The composition of any one of claims 1-20, further comprising
(c) a Cas enzyme, or a nucleic acid expression construct encoding a Cas enzyme.

22. The composition of claim 21, wherein the Cas enzyme is complexed with the CRISPR RNA.

23. The composition of claim 21 or 22, wherein the Cas enzyme is selected from spCas9, FnCas9, SaCas9, NmCas9, St1Cas9, St3Cas9, AsCPf1, LbCpf1, and Cas13.

24. The composition of any one of claims 21-23, wherein the Cas enzyme
(i) has double-strand cutting nuclease activity; or
(ii) has single strand cutting (“nickase”) activity; or
(iii) has no strand cutting (“dead”) activity;
optionally wherein the Cas enzyme is a fusion protein further comprising a heterologous polypeptide,
optionally wherein the heterologous polypeptide comprises one or more of a DNA binding domain, a deaminase domain, and a protein-binding domain.

25. The composition of any one of claims 1-24 packaged in a microparticle or nanoparticle, optionally wherein the particle is a liposome or polymeric particle.

26. The composition of any one of claims 1-25, wherein
(i) the target sequence of the spacer-sequence contain CRISPR RNA is 15-25 nucleotides, or any subrange of specific integer there between; and
(ii) the ssPNA is 5-20 PNA residues or any subrange of specific integer there
between,
wherein the ssPNA binds the spacer sequence at a site that is at least 1, 2, 3, 4, 5, 6, 7, 8, 9, 10, 11, 12, 13, 14, 15, 16, 17, 18, 19, or 20 nucleotides distal to a nucleotide that forms the protospacer adjacent motif (PAM) of the CRISPR/Cas target site.

27. The composition of any one of claims 1-26, wherein
(i) the target sequence of the spacer-sequence containing CRISPR RNA is 20 nucleotides; and
(ii) the ssPNA comprises 10 PNA residues; and
(iii) the ssPNA binds the CRISPR RNA at a site that is 0, 5, 10, 15 or 20 nucleotides distal to a nucleotide that forms the protospacer adjacent motif (PAM) of the CRISPR/Cas target site.

28. A pharmaceutical composition comprising an effective amount of the composition of any one of claims 1-27.

29. The pharmaceutical composition of claim 28, wherein the composition is in an amount effective to reduce or prevent the activity of a CRISPR/Cas complex at the CRISPR/Cas target site.

30. The pharmaceutical composition of claim 28, wherein the composition is in an amount effective to reduce activity of a CRISPR/Cas complex at an off-target site of the CRISPR/Cas target site.

31. The pharmaceutical composition of claim 30, wherein the composition is in an amount effective to reduce or eliminate Cas localization and/or activity at the off-target site, preferably while activity at the similar or related on-target site is reduced to a lesser extent or preferably is not reduced.

32. The pharmaceutical composition of any one of claims 28-31 comprising an effective amount of one or more additional single stranded anti-spacer peptide nucleic acid oligomers directed to one or more different CRISPR/Cas target sites.

33. The pharmaceutical composition of claim 32, wherein the number of additional single stranded anti-spacer peptide nucleic acid oligomers directed to one or more different CRISPR/Cas target sites is between 1 and 1,000 inclusive, or any subrange or specific integer number there between.

34. A method of reducing Cas localization and/or activity at a desired target site in the genome of a population of cells comprising contacting the cells with an effective amount of the composition of any one of claims 1-27, or the pharmaceutical composition of any one of claims 28-33.

35. The method of claim 34, wherein the ssPNA is hybridized to a spacer-sequence containing CRISPR RNA.

36. The method of claim 35, wherein the method includes a step of annealing the ssPNA to a spacer-sequence containing CRISPR RNA prior to contacting the cells with the composition or the pharmaceutical composition.

37. The method of any one of claims 34-36, wherein the desired target site is an on-target site, and Cas localization and/or activity is reduced or eliminated at the desired on-target target site.

38. The method of any one of claims 34-36, wherein the desired target site is an off-target site, and Cas localization and/or activity is reduced or eliminated at the desired off-target target site.

39. The method of claim 38, wherein Cas localization and/or activity is reduced or eliminated at the desired off-target target site but is not eliminated at the on-target site.

40. The method of claim 38 or 39, wherein Cas localization and/or activity is reduced to a lesser extent relative to the on-target site, or wherein Cas localization and/or activity is not reduced at the on-target site.

41. The method of any one of claims 34-40, wherein the desired target site comprises at least 20, 30, 40, 50, 60, 70, 75, 80, 85, 90, or 95% sequence identity to another CRISPR/Cas target site or the reverse complement thereof.

42. The method of claim 41, wherein the desired target site comprises less than 100, 95, 90, 85, 80, 75, 70, 60, 50, 40, 30, or 20% sequence identity to another CRISPR/Cas target site or the reverse complement thereof.

43. The method of any one of claims 34-42, wherein the desired target site and the CRISPR/Cas target site or its reverse complement share at least between 5-25, or any subrange or specific integer number thereof, nucleotides in common, optionally wherein the nucleotides in common are contiguous.

44. The method of any one of claims 34-43, further comprising contacting the cells with a spacer sequence containing CRISPR RNA that targets the desired CRISPR/Cas target site, or a nucleic acid expression construct encoding the same.

45. The method of any one of claims 34-44 further comprising contacting the cells with a Cas enzyme, or a nucleic acid expression construct encoding the same.

46. The method of claim 45, wherein the Cas enzyme is a fusion protein further comprising a heterologous polypeptide,
optionally wherein the heterologous polypeptide comprises a DNA binding domain, deaminase domain, or a protein-binding domain.

47. The method of claims 45 or 46, wherein the Cas enzyme modulates gene regulation, epigenetic editing, chromatin engineering, or imaging.

48. The method of any one of claims 34-47, further comprising contacting the cells with a donor oligonucleotide to recombine into the genome adjacent to the desired CRISPR/Cas target site.

49. The method of any one of claims 34-48, wherein the cells are contacted in vitro or ex vivo.

50. The method of any one of claims 34-48, wherein the cells are contacted in vivo.

51. The method of any one of claims 34-50, comprising editing the genome of the cells at or adjacent to the desired CRISPR/Cas target site.

52. The method of claim 51, wherein the editing comprises introducing a donor oligonucleotide and/or mutating existing nucleotides at or adjacent to the CRISPR/Cas target site.

53. The method of claim 52, wherein the donor oligonucleotide or mutation corrects a disease-causing nucleic acid sequence.

54. The method of any one of claims 34-53, comprising modulating gene regulation, epigenetic editing, chromatin engineering, or imaging.

55. The method of any one of claims 34-54, wherein the ssPNA is present at a ratio of ssPNA:CRISPR RNA at a molar ratio of at least 1:1.

56. The method of any one of claims 34-55, wherein the ssPNA is present in an amount between 30 pmol and 250 pmol, inclusive,
optionally wherein the ssPNA is present in an amount between 50 pmol and 150 pmol, inclusive.

57. The method of any one of claims 34-56, wherein the ssPNA is present in an amount of 150 pmol.

58. A method of reducing Cas localization and/or activity at a desired target site in the genome of a population of cells comprising contacting the cells with an effective amount of an ssPNA comprising a nucleic acid sequence of any one of the sequences set forth in Table 3 or Table 3A.

59. A CRISPR RNA/PNA duplex, comprising
(a) a spacer sequence-containing CRISPR RNA,
wherein the spacer sequence is complimentary to a protospacer sequence of a CRISPR/Cas target site within genomic DNA, and
wherein the spacer sequence comprises from 15 to 25 contiguous nucleotides, inclusive, or any subrange of specific integers there between; and
(b) a single stranded anti-spacer peptide nucleic acid oligomer (ssPNA),
wherein the ssPNA comprises 5-25 contiguous nucleobases inclusive, or any subrange of specific integers there between,
wherein at least 50%, up to 100% of the ssPNA nucleobases are complimentary to the nucleotides in the spacer sequence, and
wherein the at least 50% up to 100% of the PNA nucleobases are hybridized to the complimentary nucleotides in the spacer sequence.

60. The CRISPR RNA/PNA duplex of claim 59, wherein
(i) the ssPNA is hybridized to the spacer sequence by Watson-Crick binding only; and/or
(ii) the ssPNA is hybridized to 1, 2, 3, 4, 5, 6, 7, 8, 9, 10, 11, 12, 13 14, 15, 16, 17, 18, 19, 20, 21, 22, 23, 24 or 25 nucleotides of the CRISPR RNA spacer sequence; and/or
(iii) the hybridization comprises 0, 1, 2, 3, 4, 5 or more mismatches, gaps, and/or insertions; and/or
(iv) the ssPNA is hybridized to all the nucleotides in the spacer sequence.

61. The CRISPR RNA/PNA duplex of any one of claims 59 or 60, wherein the ssPNA comprises a nucleic acid sequence of any one of the sequences set forth in Table 3 or Table 3A.

62. A pharmaceutical composition comprising
an effective amount of the CRISPR RNA/PNA duplex of any one of claims 59-61, to reduce activity of CRISPR/Cas at the CRISPR/Cas target site.

63. A method of reducing Cas localization and/or activity at a desired CRISPR/Cas target site in the genome of a population of cells comprising contacting the cells with an effective amount of the CRISPR RNA/PNA duplex of any one of claims 59-61, or the pharmaceutical composition of claim 62.

64. Any of claims 1-63, wherein the ssPNA further comprises a non-PNA molecule conjugated or otherwise attached thereto, optionally wherein the non-PNA molecule is a targeting moiety that increases accumulation of the ssPNA in a particular tissue or cell types,
optionally wherein the targeting moiety is a sugar.


SEQUENCE LISTING (68 sequences)

SEQ ID NO: 1 - DNA, 20 bp, Artificial Sequence
  Synthetic nucleic acid
gctgaagcactgcacgccat

SEQ ID NO: 2 - DNA, 20 bp, Artificial Sequence
  Synthetic oligonucleotide
gtgaacgtggatgaagttgg

SEQ ID NO: 3 - DNA, 100 bp, Artificial Sequence
  Synthetic oligonucleotide
ctgccggtgccctggcccaccctcgtgaccaccctgacccatggcgtgcagtgcttcagccgctaccccgaccacatgaagcagcacgacttcttcaagt

SEQ ID NO: 4 - DNA, 100 bp, Artificial Sequence
  Synthetic oligonucleotide
tcctgaggagaagtctgccgttactgccctgtggggcaaggtgaacgtggatgaagttggtggtgaggccctgggcaggttggtatcaaggttacaagac

SEQ ID NO: 5 - DNA, 19 bp, Artificial Sequence
  Synthetic oligonucleotide
gcacgccctttccaaacct

SEQ ID NO: 6 - DNA, 20 bp, Artificial Sequence
  Synthetic oligonucleotide
atggcgtgcagtgcttcagc

SEQ ID NO: 7 - DNA, 21 bp, Artificial Sequence
  Synthetic oligonucleotide
gggttaaggcaatacgcaata

SEQ ID NO: 8 - DNA, 20 bp, Artificial Sequence
  Synthetic oligonucleotide
ggcctcaccaccaactgcat

SEQ ID NO: 9 - DNA, 20 bp, Artificial Sequence
  Synthetic oligonucleotide
cgctaccccgaccacatgaa

SEQ ID NO: 10 - DNA, 20 bp, Artificial Sequence
  Synthetic oligonucleotide
gtgcttcagccgctaccccg

SEQ ID NO: 11 - DNA, 20 bp, Artificial Sequence
  Synthetic oligonucleotide
accctgacccatggcgtgca

SEQ ID NO: 12 - DNA, 20 bp, Artificial Sequence
  Synthetic oligonucleotide
cctcgtgaccaccctgaccc

SEQ ID NO: 13 - DNA, 20 bp, Artificial Sequence
  Synthetic oligonucleotide
ttcatgtggtcggggtagcg

SEQ ID NO: 14 - DNA, 20 bp, Artificial Sequence
  Synthetic oligonucleotide
cggggtagcggctgaagcac

SEQ ID NO: 15 - DNA, 20 bp, Artificial Sequence
  Synthetic oligonucleotide
tgcacgccatgggtcagggt

SEQ ID NO: 16 - DNA, 20 bp, Artificial Sequence
  Synthetic oligonucleotide
gggtcagggtggtcacgagg

SEQ ID NO: 17 - DNA, 20 bp, Artificial Sequence
  Synthetic oligonucleotide
atggcgtgcagtgcttcagc

SEQ ID NO: 18 - DNA, 20 bp, Artificial Sequence
  Synthetic oligonucleotide
ggatgtttgcctaatggtgg

SEQ ID NO: 19 - DNA, 20 bp, Artificial Sequence
  Synthetic oligonucleotide
gctgcagaagggattccatg

SEQ ID NO: 20 - DNA, 19 bp, Artificial Sequence
  Synthetic oligonucleotide
aggtttggaaagggcgtgc

SEQ ID NO: 21 - DNA, 100 bp, Artificial Sequence
  Synthetic oligonucleotide
gggtggaggagagggaggtggggggagaaactgaggcgaaggatgtttgcctaatggtggtggcaatggtgtctgtggaaggggaaaaccgggagacaca

SEQ ID NO: 22 - DNA, 100 bp, Artificial Sequence
  Synthetic oligonucleotide
tgacagaggtgcctgggtttcatatgactgacttcgtattccccttggaatcccttctgcagcatagtggatgaggtgagacagaggctggtaactcggt

SEQ ID NO: 23 - DNA, 100 bp, Artificial Sequence; identical to SEQ ID NO: 22
  Synthetic oligonucleotide

SEQ ID NO: 24 - DNA, 100 bp, Artificial Sequence
  Synthetic oligonucleotide
agccagcgtttcccgcggatacagcagtcgggtgttggagaggtttggaaagggcgtgccggagagccaagtgcagccgcctagggctgccggtcgctcc

SEQ ID NO: 25 - DNA, 20 bp, Artificial Sequence
  Synthetic oligonucleotide
gctgaagcactgcacgccat

SEQ ID NO: 26 - DNA, 20 bp, Artificial Sequence
  Synthetic oligonucleotide
ccaccattaggcaaacatcc

SEQ ID NO: 27 - DNA, 20 bp, Artificial Sequence
  Synthetic oligonucleotide
gacttcgtattccccttgga

SEQ ID NO: 28 - DNA, 10 bp, Artificial Sequence
  Synthetic oligonucleotide
cttctgcagc

SEQ ID NO: 29 - DNA, 20 bp, Artificial Sequence
  Synthetic oligonucleotide
atggcgtgcagtgcttcagc

SEQ ID NO: 30 - DNA, 10 bp, Artificial Sequence
  Synthetic oligonucleotide
atggcgtgca

SEQ ID NO: 31 - DNA, 10 bp, Artificial Sequence
  Synthetic oligonucleotide
gtgcagtgct

SEQ ID NO: 32 - DNA, 10 bp, Artificial Sequence
  Synthetic oligonucleotide
gtgcttcagc

SEQ ID NO: 33 - DNA, 20 bp, Artificial Sequence
  Synthetic oligonucleotide
ccaacttcatccacgttcac

SEQ ID NO: 34 - DNA, 20 bp, Artificial Sequence
  Synthetic oligonucleotide
accctgacccatggcgtgca

SEQ ID NO: 35 - DNA, 20 bp, Artificial Sequence
  Synthetic oligonucleotide
gtgcttcagccgctaccccg

SEQ ID NO: 36 - DNA, 20 bp, Artificial Sequence
  Synthetic oligonucleotide
cgctaccccgaccacatgaa

SEQ ID NO: 37 - DNA, 20 bp, Artificial Sequence
  Synthetic oligonucleotide
cctcgtgaccaccctgaccc

SEQ ID NO: 38 - DNA, 20 bp, Artificial Sequence
  Synthetic oligonucleotide
gctgaagcactgcacgccat

SEQ ID NO: 39 - DNA, 20 bp, Artificial Sequence
  Synthetic oligonucleotide
cggggtagcggctgaagcac

SEQ ID NO: 40 - DNA, 20 bp, Artificial Sequence
  Synthetic oligonucleotide
ttcatgtggtcggggtagcg

SEQ ID NO: 41 - DNA, 20 bp, Artificial Sequence
  Synthetic oligonucleotide
gggtcagggtggtcacgagg

SEQ ID NO: 42 - DNA, 20 bp, Artificial Sequence
  Synthetic oligonucleotide
tgcacgccatgggtcagggt

SEQ ID NO: 43 - DNA, 20 bp, Artificial Sequence
  Synthetic oligonucleotide
gggttaaggcaatagcaata

SEQ ID NO: 44 - DNA, 10 bp, Artificial Sequence
  Synthetic oligonucleotide
atggcgtgca

SEQ ID NO: 45 - DNA, 10 bp, Artificial Sequence
  Synthetic oligonucleotide
gtgcagtgct

SEQ ID NO: 46 - DNA, 10 bp, Artificial Sequence
  Synthetic oligonucleotide
gtgcttcagc

SEQ ID NO: 47 - DNA, 10 bp, Artificial Sequence
  Synthetic oligonucleotide
ccaactgcat

SEQ ID NO: 48 - DNA, 10 bp, Artificial Sequence
  Synthetic oligonucleotide
cttctgcagc

SEQ ID NO: 49 - DNA, 10 bp, Artificial Sequence
  Synthetic oligonucleotide
cttgccgcac

SEQ ID NO: 50 - DNA, 10 bp, Artificial Sequence
  Synthetic oligonucleotide
ctcactcacc

SEQ ID NO: 51 - DNA, 10 bp, Artificial Sequence
  Synthetic oligonucleotide
tccgctctac

SEQ ID NO: 52 - DNA, 20 bp, Artificial Sequence
  Synthetic oligonucleotide
ccaccattaggcaaacatcc

SEQ ID NO: 53 - DNA, 19 bp, Artificial Sequence
  Synthetic oligonucleotide
gcacgccctttccaaacct

SEQ ID NO: 54 - DNA, 10 bp, Artificial Sequence
  Synthetic oligonucleotide
gctcggactc

SEQ ID NO: 55 - DNA, 20 bp, Artificial Sequence
  Synthetic oligonucleotide
gtagagcggaggcaggaggc

SEQ ID NO: 56 - DNA, 20 bp, Artificial Sequence
  Synthetic oligonucleotide
ggtgagtgagtgtgtgcgtg

SEQ ID NO: 57 - DNA, 20 bp, Artificial Sequence
  Synthetic oligonucleotide
gtgcggcaagagcttcagcc

SEQ ID NO: 58 - DNA, 20 bp, Artificial Sequence
  Synthetic oligonucleotide
gagtccgagcagaagaagaa

SEQ ID NO: 59 - DNA, 127 bp, Artificial Sequence
  Synthetic oligonucleotide
tcatgtggtcggggtagcggctgaagcactgcacgccgtacgtcagggtggtcacgagggtgggccagggcacgggcagcttgccggtggtgcagatgaacttcagggtcagcttgccgtaggtggc

SEQ ID NO: 60 - DNA, 24 bp, Artificial Sequence
  Synthetic oligonucleotide
tctctcacgacctgatttctacag

SEQ ID NO: 61 - DNA, 24 bp, Artificial Sequence
  Synthetic oligonucleotide
cacctctgagctgactttcaatac

SEQ ID NO: 62 - DNA, 20 bp, Artificial Sequence
  Synthetic oligonucleotide
atggcgtgcagtgcttcagc

SEQ ID NO: 63 - RNA, 20 nt, Artificial Sequence
  Synthetic oligonucleotide
auggcgugcagugcuucagc

SEQ ID NO: 64 - RNA, 20 nt, Artificial Sequence
  Synthetic oligonucleotide
gcugaagcacugcacgccau

SEQ ID NO: 65 - DNA, 20 bp, Artificial Sequence
  Synthetic oligonucleotide
tcagctcagcctgagtgttg

SEQ ID NO: 66 - DNA, 20 bp, Artificial Sequence
  Synthetic oligonucleotide
ttgcccaccctagtcattgg

SEQ ID NO: 67 - DNA, 20 bp, Artificial Sequence
  Synthetic oligonucleotide
cccgcttgtccatgtctagg

SEQ ID NO: 68 - DNA, 23 bp, Artificial Sequence
  Synthetic oligonucleotide
agaaatgcccaatcattgatgct